(19) United States
(12) Patent Application Publication
(10) Pub. No.: US 2021/0355115 A1
(43) Pub. Date: Nov. 18, 2021

(54) ISOXAZOLE CARBOXAMIDE COMPOUNDS AND USES THEREOF

(71) Applicant: Novartis AG, Basel (CH)

(72) Inventors: Rohan Eric John Beckwith, Acton, MA (US); Hua Jiang, Shanghai (CN); Ce Wang, Shanghai (CN)

(21) Appl. No.: 17/278,163

(22) PCT Filed: Sep. 19, 2019
(86) PCT No.: PCT/IB2019/057941

§ 371 Date: Mar. 19, 2021

(30) Foreign Application Priority Data
Sep. 21, 2018 (CN) PCT/CN2018/106939

(51) Int. Cl. C07D 413/14; A61P 27/16

(52) U.S. Cl. CPC C07D 413/14; A61P 27/16

(57) ABSTRACT

A compound of Formula (I) or or a pharmaceutically acceptable salt thereof, is provided that has been shown to be useful for treating hearing loss or balance disorder: Formula (I) wherein R¹ and Y are as defined herein.

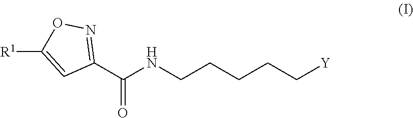

### CROSS-REFERENCE TO RELATED APPLICATION

This application claims the benefit of priority to International Application Patent Application No. PCT/CN2018/106939, filed 21 Sep. 2018, the contents of which is incorporated herein by reference.

### FIELD OF THE INVENTION

The present disclosure relates to compounds, compositions comprising such compounds, and their use for the treatment of hearing loss or balance disorder.

### BACKGROUND OF THE INVENTION

Hair cells in the inner ear are essential for hearing and balance. If hair cells are damaged in any way, human beings would suffer hearing loss or balance disorder. The human inner ear contains only about 15,000 hair cells per cochlea at birth, and, although these cells can be lost as a result of various genetic or environmental factors, the lost or damaged cells cannot be replaced. However, overexpression of the transcription factor, Atoh1, can induce sensory hair cells from epithelial cells in the sensory organ of the cochlea and the organ of Corti (Zheng and Gao, Nat Neurosci 2000; 3:580-586; Kawamoto et al., J Neurosci 2003; 23:4395-4400; Izumikawa M et al., Nat Med. 2005; 11: 271-276; Gubbels et al., Nature 2008; 455:537-541). Therefore, there is a need to discover therapeutic compositions and methods that induce Atoh1 expression and promote mammalian hair cell regeneration.

### SUMMARY OF THE INVENTION

The present disclosure provides compounds, pharmaceutically acceptable salts thereof, pharmaceutical compositions thereof and combinations thereof, which are useful to treat hearing loss or balance disorder. The present disclosure further provides methods of treating hearing loss or balance disorder, comprising administering to a subject in need thereof an effective amount of a compound of the present disclosure, or a pharmaceutically acceptable salt thereof.

One aspect of the present disclosure provides a compound of Formula (I) or a pharmaceutically acceptable salt thereof:

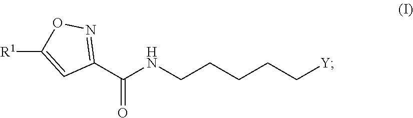

wherein:

R¹ is selected from:

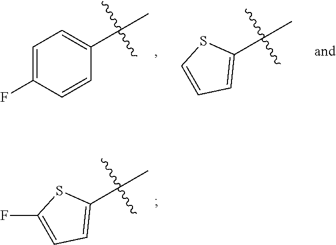 [SMILES: *c1ccc(F)cc1 ; *c1cccs1 ; *c1ccc(F)s1]

Y is selected from

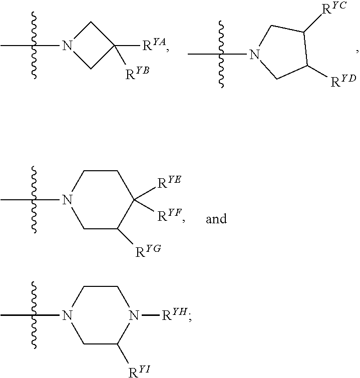

R^{YA} and R^{YB} are independently selected from H, —S(═O)₂NH(R²), —(C═O)NH(R²), —NH(C═O)OCH₂(C═O)NH(R²), —CH₂OH, —CH₃, and —OH;

R^{YC} and R^{YA} are independently selected from H, —CN, —OH, —(C═O)NH₂, and —S(═O)₂NH(R²);

R^{YE} and R^{YF} are independently selected from H, —CN, —(C═O)NH(R²), —OH, and —S(═O)₂NH(R²);

R^{YG} is selected from H, —CN, —OH, —F, —(C═O)NH(R²), and —S(═O)₂NH(R²);

R^{YH} is selected from —CH₃, —(X¹)—(C═O)NH(R²), and —(X¹)—S(═O)₂NH(R²);

R^{YI} is selected from —H and —(C═O)NH(R²);

X¹ is C₀₋₂alkylene, optionally substituted with —OH;

R² is independently selected from H, —CH₃, —CH(CH₃)CN, —CH₂CH₂CN,

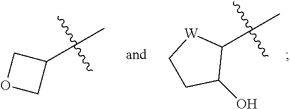

and

W is O or CH₂.

Another aspect of the present disclosure provides a pharmaceutical composition comprising a therapeutically effective amount of a compound of Formula (I) or a pharmaceutically acceptable salt thereof, or subformulae thereof, and one or more pharmaceutically acceptable carriers.

In yet another aspect of present disclosure, a pharmaceutical combination is provided which comprises a therapeutically effective amount of a compound of Formula (I) or a pharmaceutically acceptable salt thereof, or subformulae thereof, and one or more therapeutically active agents.

In yet another aspect of present disclosure, a method is provided for treating hearing loss or balance disorder, which comprises administering to a subject in need thereof a therapeutically effective amount of a compound of Formula (I) or a pharmaceutically acceptable salt thereof, or subformulae thereof.

In yet another aspect of the present disclosure, processes are provided for preparing compounds of Formula (I) or a pharmaceutically acceptable salt thereof, or subformulae thereof.

### DETAILED DESCRIPTION

Various (enumerated) embodiments of the disclosure are described herein. It will be recognized that features specified in each embodiment may be combined with other specified features to provide further embodiments of the present disclosure.

Embodiment 1: A compound of Formula (I)

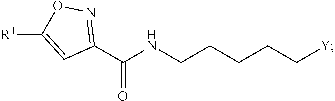

or a pharmaceutically acceptable salt thereof,
wherein:

R¹ is selected from:

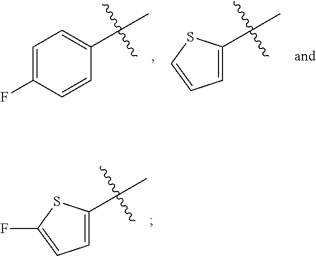 [SMILES: *c1ccc(F)cc1 ; *c1cccs1 ; *c1ccc(F)s1]

Y is selected from

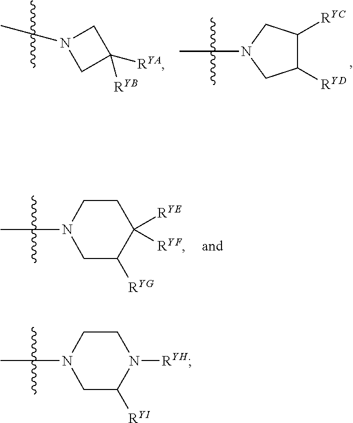

R^{YA} and R^{YB} are independently selected from H, —S(═O)₂NH(R²), —(C═O)NH(R²), —NH(C═O)OCH₂(C═O)NH(R²), —CH₂OH, —CH₃, and —OH;

R^{YC} and R^{YA} are independently selected from H, —CN, —OH, —(C═O)NH₂, and —S(═O)₂NH(R²);

R^{YE} and R^{YF} are independently selected from H, —CN, —(C═O)NH(R²), —OH, and —S(═O)₂NH(R²);

R^{YG} is selected from H, —CN, —OH, —F, —(C═O)NH(R²), and —S(═O)₂NH(R²);

R^{YH} is selected from —CH₃, —(X¹)—(C═O)NH(R²), and —(X¹)—S(═O)₂NH(R²);

R^{YI} is selected from —H and —(C═O)NH(R²);

X¹ is C₀₋₂alkylene, optionally substituted with —OH;

R² is independently selected from H, —CH₃, —CH(CH₃)CN, —CH₂CH₂CN,

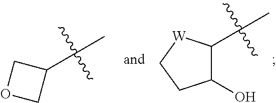

and

W is O or CH₂;

wherein when R¹ is:

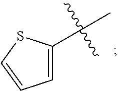 [SMILES: *c1cccs1]

### Y is not:

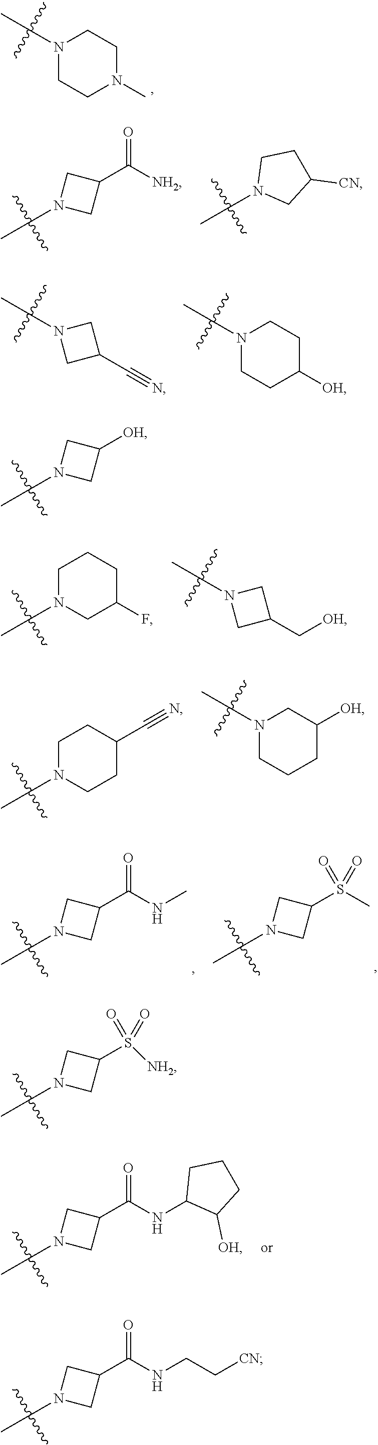

when R¹ is:

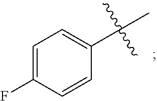 [SMILES: *c1ccc(F)cc1]

### Y is not:

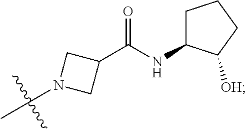 [SMILES: *N1CC(C(=O)N[C@H]2CCC[C@@H]2O)C1]

and
when R¹ is:

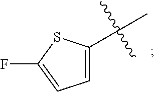 [SMILES: *c1ccc(F)s1]

### Y is not:

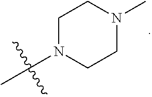 [SMILES: *N1CCN(C)CC1]

Embodiment 2: A compound of Formula (I)

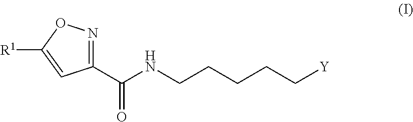

or a pharmaceutically acceptable salt thereof,
wherein:

R¹ is selected from:

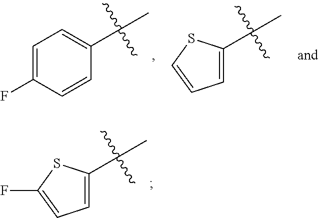 [SMILES: *c1ccc(F)cc1 ; *c1cccs1 ; *c1ccc(F)s1]

Y is selected from

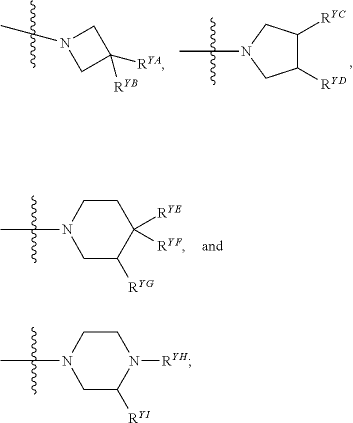

R^{YA} and R^{YB} is independently selected from H, —S(═O)₂NH(R²), —(C═O)NH(R²), —NH(C═O)OCH₂(C═O)NH(R²), —CH₂OH, —CH₃, and —OH;

R^{YC} and R^{YA} is independently selected from H, —CN, —OH, —(C═O)NH₂, and —S(═O)₂NH(R²);

R^{YE} and R^{YF} is independently selected from H, —CN, —(C═O)NH(R²), —OH, and —S(═O)₂NH(R²);

R^{YG} is selected from H, —CN, —OH, —F, —(C═O)NH(R²), and —S(═O)₂NH(R²);

R^{YH} is selected from —CH₃, —(X¹)—(C═O)NH(R²), and —(X¹)—S(═O)₂NH(R²);

R^{YI} is selected from —H and —(C═O)NH(R²);

X¹ is C₀₋₂alkylene, optionally substituted with —OH;

R² is independently selected from H, —CH₃, —CH(CH₃)CN, —CH₂CH₂CN,

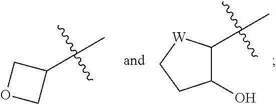

and

### W is O or CH₂;

wherein the compound is not:

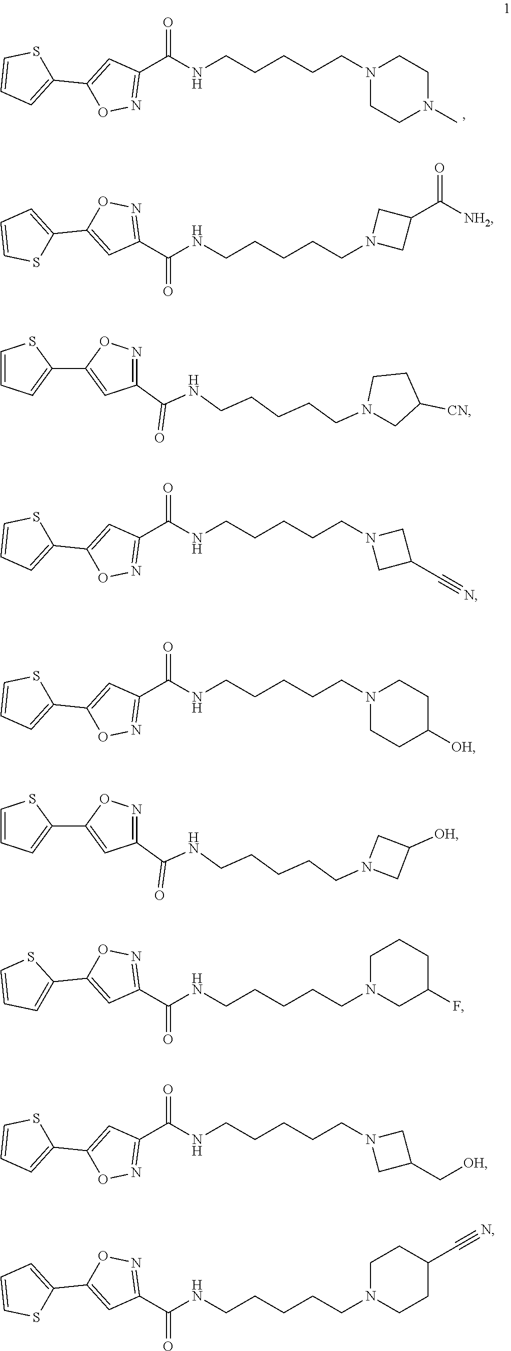

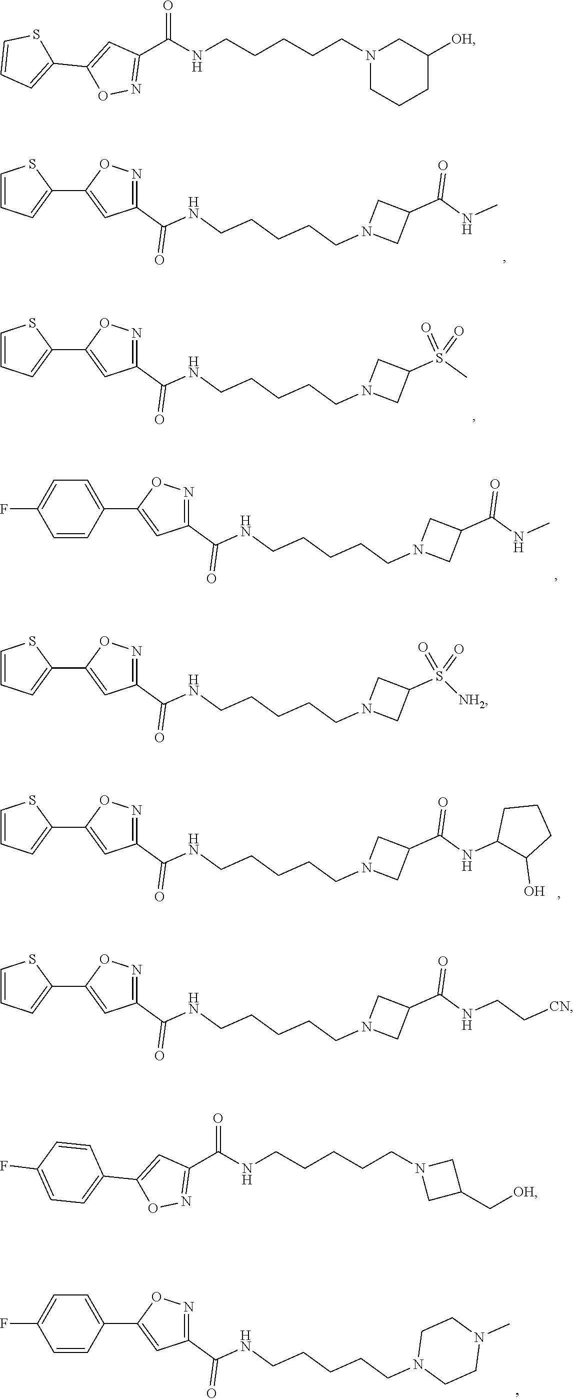

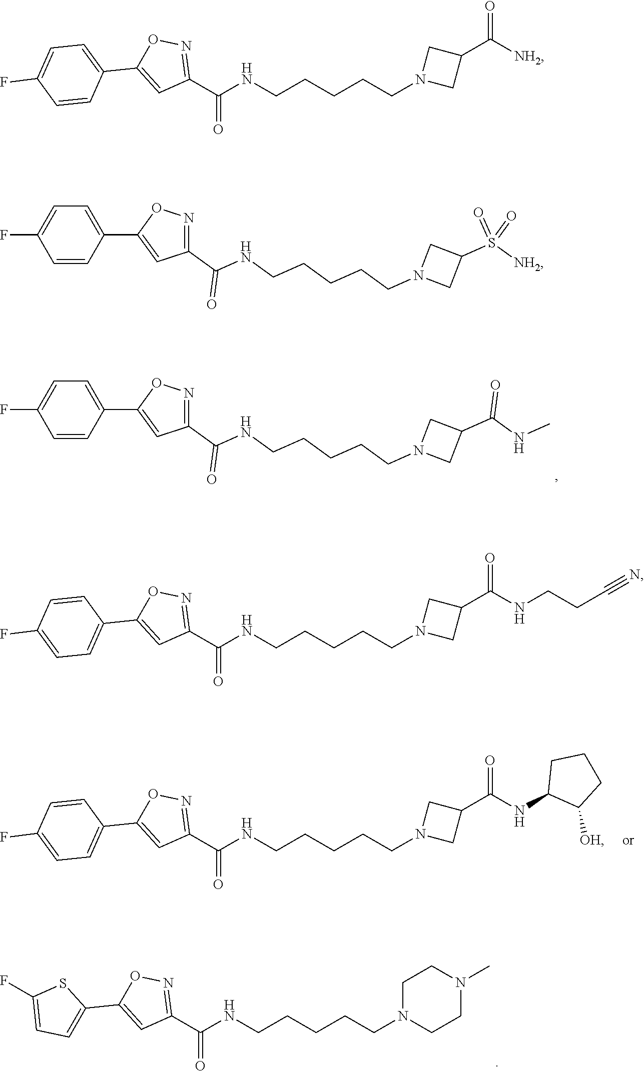 [SMILES: NC(=O)C1CN(CCCCCNC(=O)c2cc(-c3ccc(F)cc3)on2)C1 ; NS(=O)(=O)C1CN(CCCCCNC(=O)c2cc(-c3ccc(F)cc3)on2)C1 ; CNC(=O)C1CN(CCCCCNC(=O)c2cc(-c3ccc(F)cc3)on2)C1 ; N#CCCNC(=O)C1CN(CCCCCNC(=O)c2cc(-c3ccc(F)cc3)on2)C1 ; O=C(NCCCCCN1CC(C(=O)N[C@H]2CCC[C@@H]2O)C1)c1cc(-c2ccc(F)cc2)on1 ; CN1CCN(CCCCCNC(=O)c2cc(-c3ccc(F)s3)on2)CC1]

Embodiment 3: A compound of claim 1 or 2 or a pharmaceutically acceptable salt thereof, wherein:

R^{YG} is selected from H, —CN, —OH, —F, —(C═O)NH₂, and —S(═O)₂NH₂;
R^{YH} is selected from —CH₃, —(X¹)—(C═O)NH₂, and —(X¹)—S(═O)₂NH₂;
R^{YI} is selected from —H and —(C═O)NH₂;
X¹ is C₁₋₂alkylene, optionally substituted with OH.

Embodiment 4: A compound of any one of embodiments 1-3 or a pharmaceutically acceptable salt thereof, wherein:

R¹ is:

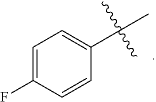 [SMILES: *c1ccc(F)cc1]

Embodiment 5: A compound of any one of embodiments 1-3 or a pharmaceutically acceptable salt thereof, wherein:
R¹ is:

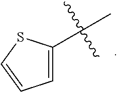 [SMILES: *c1cccs1]

Embodiment 6: A compound of any one of embodiments 1-3 or a pharmaceutically acceptable salt thereof, wherein:

R¹ is:

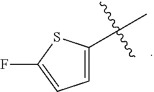 [SMILES: *c1ccc(F)s1]

Embodiment 7: A compound of any one of embodiments 1-3 or a pharmaceutically acceptable salt thereof, wherein:

### Y is

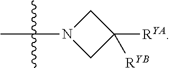

Embodiment 8: A compound of any one of embodiments 1-3 or a pharmaceutically acceptable salt thereof, wherein:

### Y is

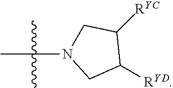

Embodiment 9: A compound of any one of embodiments 1-3 or a pharmaceutically acceptable salt thereof, wherein:

### Y is

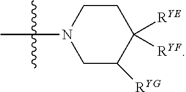

Embodiment 10: A compound of any one of embodiments 1-3 or a pharmaceutically acceptable salt thereof, wherein:

### Y is

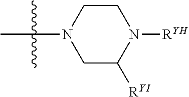

Embodiment 11: A compound or a pharmaceutically acceptable salt thereof according to Embodiment 1 selected from: N-(5-((3S,4S)-4-carbamoyl-3-cyanopiperidin-1-yl)pentyl)-5-(thiophen-2-yl)isoxazole-3-carboxamide; N-(5-((3 S,4R)-4-cyano-3-hydroxypiperidin-1-yl)pentyl)-5-(4-fluorophenyl)isoxazole-3-carboxamide; N-(5-(3-(N-(oxetan-3-yl)sulfamoyl)azetidin-1-yl)pentyl)-5-(thiophen-2-yl)isoxazole-3-carboxamide; N-(5-(3-(N-(2-cyanoethyl)sulfamoyl)azetidin-1-yl)pentyl)-5-(thiophen-2-yl)isoxazole-3-carboxamide; 2-(methylamino)-2-oxoethyl (1-(5-(5-(thiophen-2-yl)isoxazole-3-carboxamido)pentyl)azetidin-3-yl)carbamate; N-(5-(3-sulfamoylpiperidin-1-yl)pentyl)-5-(thiophen-2-yl)isoxazole-3-carboxamide; N-(5-(3-sulfamoylpyrrolidin-1-yl)pentyl)-5-(thiophen-2-yl)isoxazole-3-carboxamide; N-(5-(3-carbamoylpyrrolidin-1-yl)pentyl)-5-(4-fluorophenyl)isoxazole-3-carboxamide; N-(5-(3-carbamoyl-3-(hydroxymethyl)azetidin-1-yl)pentyl)-5-(thiophen-2-yl)isoxazole-3-carboxamide; N-(5-(3-carbamoyl-3-(hydroxymethyl)azetidin-1-yl)pentyl)-5-(4-fluorophenyl)isoxazole-3-carboxamide; N-(5-(3-carbamoyl-3-methylazetidin-1-yl)pentyl)-5-(thiophen-2-yl)isoxazole-3-carboxamide; N-(5-(3-carbamoyl-3-methylazetidin-1-yl)pentyl)-5-(4-fluorophenyl)isoxazole-3-carboxamide; N-(5-(3-carbamoyl-4-hydroxypyrrolidin-1-yl)pentyl)-5-(thiophen-2-yl)isoxazole-3-carboxamide; N-(5-(4-sulfamoylpiperidin-1-yl)pentyl)-5-(thiophen-2-yl)isoxazole-3-carboxamide; 5-(4-fluorophenyl)-N-(5-(4-sulfamoylpiperidin-1-yl)pentyl)isoxazole-3-carboxamide; N-(5-((3R,4R)-4-cyano-3-hydroxypiperidin-1-yl)pentyl)-5-(thiophen-2-yl)isoxazole-3-carboxamide; N-(5-(4-(3-amino-3-oxopropyl)piperazin-1-yl)pentyl)-5-(thiophen-2-yl)isoxazole-3-carboxamide; N-(5-(4-(2-amino-2-oxoethyl)piperazin-1-yl)pentyl)-5-(thiophen-2-yl)isoxazole-3-carboxamide; N-(5-(4-(2-sulfamoylethyl)piperazin-1-yl)pentyl)-5-(thiophen-2-yl)isoxazole-3-carboxamide; N-(5-(4-carbamoylpiperidin-1-yl)pentyl)-5-(thiophen-2-yl)isoxazole-3-carboxamide; N-(5-(3-carbamoyl-3-hydroxyazetidin-1-yl)pentyl)-5-(thiophen-2-yl)isoxazole-3-carboxamide; 5-(5-fluorothiophen-2-yl)-N-(5-(3-(methylcarbamoyl)azetidin-1-yl)pentyl)isoxazole-3-carboxamide; N-(5-(3-carbamoylazetidin-1-yl)pentyl)-5-(5-fluorothiophen-2-yl)isoxazole-3-carboxamide; N-(5-(3-((1-cyanoethyl)carbamoyl)azetidin-1-yl)pentyl)-5-(4-fluorophenyl)isoxazole-3-carboxamide; N-(5-(3-carbamoyl-4-methylpiperazin-1-yl)pentyl)-5-(thiophen-2-yl)isoxazole-3-carboxamide; N-(5-(4-carbamoyl-4-hydroxypiperidin-1-yl)pentyl)-5-(thiophen-2-yl)isoxazole-3-carboxamide; 5-(4-fluorophenyl)-N-(5-(3-(((1S,2R)-2-hydroxycyclopentyl)carbamoyl)azetidin-1-yl)pentyl)isoxazole-3-carboxamide; N-(5-((3R,4S)-4-carbamoyl-3-fluoropiperidin-1-yl)pentyl)-5-(thiophen-2-yl)isoxazole-3-carboxamide, and N-(5-((3 S,4S)-4-carbamoyl-3-fluoropiperidin-1-yl)pentyl)-5-(thiophen-2-yl)isoxazole-3-carboxamide; N-(5-((3R,4S)-3-carbamoyl-4-cyanopiperidin-1-yl)pentyl)-5-(thiophen-2-yl)isoxazole-3-carboxamide; N-(5-(4-carbamoyl-3-hydroxypiperidin-1-yl)pentyl)-5-(thiophen-2-yl)isoxazole-3-carboxamide; N-(5-(4-carbamoyl-3-hydroxypiperidin-1-yl)pentyl)-5-(4-fluorophenyl)isoxazole-3-carboxamide; N-(5-((3S,4S)-3-carbamoyl-4-hydroxypyrrolidin-1-yl)pentyl)-5-(4-fluorophenyl)isoxazole-3-carboxamide; N-(5-((3 S,4R)-3-carbamoyl-4-hydroxypyrrolidin-1-yl)pentyl)-5-(4-fluorophenyl)isoxazole-3-carboxamide; N-(5-((3R,4S)-3-carbamoyl-4-hydroxypyrrolidin-1-yl)pentyl)-5-(4-fluorophenyl)isoxazole-3-carboxamide; N-(5-((3R,4R)-3-carbamoyl-4-hydroxypyrrolidin-1-yl)pentyl)-5-(4-fluorophenyl)isoxazole-3-carboxamide; N-(5-((3S,4S)-3-carbamoyl-4-hydroxypyrrolidin-1-yl)pentyl)-5-(thiophen-2-yl)isoxazole-3-carboxamide; N-(5-((3R,4S)-3-carbamoyl-4-hydroxypyrrolidin-1-yl)pentyl)-5-(thiophen-2-yl)isoxazole-3-carboxamide; N-(5-((3R,4R)-3-carbamoyl-4-hydroxypyrrolidin-1-yl)pentyl)-5-(thiophen-2-yl)isoxazole-3-carboxamide; N-(5-((3 S,4R)-3-carbamoyl-4-hydroxypyrrolidin-1-yl)pentyl)-5-(thiophen-2-yl)isoxazole-3-carboxamide; N-(5-((3 S,4R)-3-cyano-4-hydroxypyrrolidin-1-yl)pentyl)-5-(thiophen-2-yl)isoxazole-3-carboxamide, and N-(5-((3S,4S)-3-cyano-4-hydroxypyrrolidin-1-yl)pentyl)-5-(thiophen-2-yl)isoxazole-3-carboxamide; N-(5-((3R,4S)-3-carbamoyl-4-hydroxypiperidin-1-yl)pentyl)-5-(thiophen-2-yl)isoxazole-3-carboxamide and N-(5-((3R,4R)-3-carbamoyl-4-hydroxypiperidin-1-yl)pentyl)-5-(thiophen-2-yl)isoxazole-3-carboxamide; N-(5-(3-((4-hydroxytetrahydrofuran-3-yl)carbamoyl)azetidin-1-yl)pentyl)-5-(thiophen-2-yl)isoxazole-3-carboxamide; N-(5-(4-(3-amino-2-hydroxy-3-oxopropyl)piperazin-1-yl)pentyl)-5-(thiophen-2-yl)isoxazole-3-carboxamide.

Embodiment 12: A compound or a pharmaceutically acceptable salt thereof, according to Embodiment 1, wherein said compound is selected from any one or more exemplified examples.

Embodiment 13: A pharmaceutical composition, comprising:

a therapeutically effective amount of a compound of Formula (I) according to any one of the Embodiments 1-12 or a pharmaceutically acceptable salt thereof, and one or more pharmaceutically acceptable carriers.

Embodiment 14: A pharmaceutical combination, comprising:

a therapeutically effective amount of a compound of Formula (I) according to any one of the Embodiments 1-12 or a pharmaceutically acceptable salt thereof, and one or more therapeutically active agents.

Embodiment 15: A method of treating hearing loss or balance disorder, comprising administering to a subject in need thereof a therapeutically effective amount of a compound according to any one of the Embodiments 1-12 or a pharmaceutically acceptable salt thereof.

Embodiment 16: A method according to Embodiment 15, wherein the subject has a partial or complete loss of hearing.

Embodiment 17: A method according to Embodiment 15 or 16, wherein the hearing loss is acquired hearing loss.

Embodiment 18: A method according to any one of the Embodiments 15-17, wherein the hearing loss is sensorineural hearing loss.

Embodiment 19: A method according to any one of the Embodiments 15-18, wherein the hearing loss or balance disorder is associated with damage or loss of sensory hair cells.

Embodiment 20: A method according to any one of the Embodiments 15-19, wherein the hearing loss or balance disorder is caused by acute or chronic exposure to ototoxic compounds, acute or chronic exposure to noise, aging, autoimmune disease, physical trauma, inflammation or virus.

Embodiment 21: A method according to any one of the Embodiments 15-20, wherein the compound or a pharmaceutically acceptable salt thereof, promotes, stimulates or induces sensory hair cells regeneration.

Embodiment 22: A compound according to any one of the Embodiments 1-12, or a pharmaceutically acceptable salt thereof, for use as a medicament.

Embodiment 23: A use of a compound according to any one of Embodiments 1-12, or a pharmaceutically acceptable salt thereof, in the manufacture of a medicament for the treatment of hearing loss or balance disorder.

Other features of the present disclosure should become apparent in the course of the above descriptions of exemplary embodiments that are given for illustration of the disclosure and are not intended to be limiting thereof.

### Definitions

For purposes of interpreting this specification, the following definitions will apply, and whenever appropriate, terms used in the singular will also include the plural. Terms used in the specification have the following meanings unless the context clearly indicates otherwise.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the present disclosure otherwise claimed.

The term “a,” “an,” “the” and similar terms used in the context of the present disclosure (especially in the context of the claims) are to be construed to cover both the singular and plural unless otherwise indicated herein or clearly contradicted by the context.

As used herein, the term “heteroatoms” refers to nitrogen (N), oxygen (O) or sulfur (S) atoms, in particular nitrogen or oxygen.

Unless otherwise indicated, any heteroatom with unsatisfied valences is assumed to have hydrogen atoms sufficient to satisfy the valences.

As used herein, the terms “alkyl” refers to a hydrocarbon radical of the general formula CₙH₂ₙ₊₁. The alkane radical may be straight or branched. For example, the term “C₁-C₆ alkyl” or “C₁ to C₆ alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like).

The term “C₀-C₆ alkylene” refers to a bond (when the number of carbon atom is 0) or a divalent alkylene group (may be straight or branched) containing 1 to 6 carbon atoms (e.g., methylene (—CH₂—), ethylene (—CH₂CH₂—), n-propylene (—CH₂CH₂CH₂—), iso-propylene (—CH(CH₃)CH₂—), n-butylene (—CH₂CH₂CH₂CH₂—), iso-butylene, tert-butylene, n-pentylene, isopentylene, neopentylene, n-hexylene and the like).

The term “alkoxy” refers to an alkyl linked to an oxygen, which may also be represented as —O—R or —OR, wherein the R represents the alkyl group. “C₁-C₆ alkoxy” or “C₁ to C₆ alkoxy” is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

“Halogen” or “halo” may be fluorine, chlorine, bromine or iodine (preferred halogens as substituents are fluorine and chlorine).

“Haloalkyl” is intended to include both branched and straight-chain saturated aliphatic hydrocarbon groups having the specified number of carbon atoms, substituted with one or more halogens. Thus, “C₁-C₆ haloalkyl” or “C₁ to C₆ haloalkyl” is intended to include, but not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl.

“Haloalkoxy” represents a haloalkyl group as defined above with the indicated number of carbon atoms attached through an oxygen bridge. For example, “C₁-C₆ haloalkoxy” or “C₁ to C₆ haloalkoxy” is intended to include, but not limited to, trifluoromethoxy, difluoromethoxy, 2,2,2-trifluoroethoxy, and pentafluorothoxy. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—.

The term “cycloalkyl” refers to nonaromatic carbocyclic ring that is fully hydrogenated ring, including mono-, bi- or poly-cyclic ring systems having the specified number of carbon atoms. Thus, “C₃-C₈ cycloalkyl” or “C₃ to C₈ cycloalkyl” is intended to include, but not limited to, cyclopropyl, cyclobutyl, cyclopentyl, cyclohexyl, and norbornyl.

The term “aryl” refers to 6- to 10-membered aromatic carbocyclic moieties having a single (e.g., phenyl) or a fused ring system (e.g., naphthalene.). A typical aryl group is phenyl group.

The term “heteroaryl” refers to aromatic moieties containing at least one heteroatom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, benzopyranyl, benzothiophenyl, benzoimidazolyl, benzoxazolyl, 1H-benzo[d][1,2,3]triazolyl, and the like.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a hetereoaryl fused to an aryl (e.g., phenyl).

The term “heterocyclyl” refers to a saturated or partially saturated, but not aromatic, ring or ring systems, which include a monocyclic ring, fused rings, bridged rings and spirocyclic rings having the specified number of ring atoms. For example, heterocyclyl includes, but not limited to, 5- to 6-membered heterocyclyl, 4- to 10-membered heterocyclyl, 4- to 14-membered heterocyclyl and 5- to 14-membered heterocyclyl. Unless otherwise specified, the heterocyclyl contain 1 to 7, 1 to 5, 1 to 3, or 1 to 2 heteroatoms independently selected from the group consisting of nitrogen, oxygen and sulphur as ring members, where the N and S can also optionally be oxidized to various oxidation states. The heterocyclic group can be attached at a heteroatom or a carbon atom. Examples of such heterocyclyl include, but are not limited to, azetidine, oxetane, piperidine, piperazine, pyrroline, pyrrolidine, imidazolidine, imidazoline, morpholine, tetrahydrofuran, tetrahydrothiophene, tetrahydrothiopyran, tetrahydropyran, 1,4-dioxane, 1,4-oxathiane, hexahydropyrimidinyl, 3-azabicyclo[3.1.0]hexane, azepane, 3-azabicyclo[3.2.2]nonane, decahydroisoquinoline, 2-azaspiro[3.3]heptane, 2-oxa-6-azaspiro[3.3]heptane, 2,6-diazaspiro[3.3]heptane, 8-aza-bicyclo[3.2.1]octane, 3,8-diazabicyclo[3.2.1]octane, 3-Oxa-8-aza-bicyclo[3.2.1]octane, 8-Oxa-3-aza-bicyclo[3.2.1]octane, 2-Oxa-5-aza-bicyclo[2.2.1]heptane, 2,5-Diaza-bicyclo[2.2.1]heptane, 1,4-dioxa-8-aza-spiro[4.5]decane, 3-oxa-1,8-diazaspiro[4.5]decane, octahydropyrrolo[3,2-b]pyrrol, and the like.

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown and claimed nitrogen atoms are considered to cover both the shown nitrogen and its N-oxide (NO) derivative.

When any variable occurs more than one time in any constituent or formula for a compound, its definition at each occurrence is independent of its definition at every other occurrence. Thus, for example, if a group is shown to be substituted with 0-3 R groups, then said group may be unsubstituted or substituted with up to three R groups, and at each occurrence R is selected independently from the definition of R.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent.

Combinations of substituents and/or variables are permissible only if such combinations result in stable compounds.

As a person of ordinary skill in the art would be able to understand, for example, a ketone (—CH—C═O) group in a molecule may tautomerize to its enol form (—C═C—OH). Thus, this disclosure is intended to cover all possible tautomers even when a structure depicts only one of them.

The phrase “pharmaceutically acceptable” indicates that the substance or composition must be compatible chemically and/or toxicologically, with the other ingredients comprising a formulation, and/or the mammal being treated therewith.

Unless specified otherwise, the term “compounds of the present disclosure” refers to compounds of Formula (I) and subformulae thereof, as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

It will be recognized by those skilled in the art that the compounds of the present disclosure may contain chiral centers and as such may exist in different isomeric forms. As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Calm-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Geometric isomers may occur when a compound contains a double bond or some other feature that gives the molecule a certain amount of structural rigidity. If the compound contains a double bond, the substituent may be E or Z configuration. If the compound contains a disubstituted cycloalkyl, the cycloalkyl substituent may have a cis- or trans-configuration.

Conformational isomers (or conformers) are isomers that can differ by rotations about one or more a bonds. Rotamers are conformers that differ by rotation about only a single a bond.

The term “atropisomer” refers to a structural isomer based on axial or planar chirality resulting from restricted rotation in the molecule.

Unless specified otherwise, the compounds of the present disclosure are meant to include all such possible isomers, including racemic mixtures, optically pure forms and intermediate mixtures. Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. or other equivalent columns, using the appropriate solvent or mixture of solvents to achieve good separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated within the scope of the present disclosure.

As used herein, “pharmaceutically acceptable salts” refer to derivatives of the disclosed compounds wherein the parent compound is modified by making acid or base salts thereof. For example, pharmaceutically acceptable salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate or xinafoate salt form.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence the present disclosure further provides co-crystals comprising a compound of the present disclosure.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and idodine, such as ²H, ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation) or at least 6633.3 (99.5% deuterium incorporation).

Isotopically labeled compounds of this present disclosure can generally be prepared by conventional techniques known to those skilled in the art or by processes disclosed in the schemes or in the examples and preparations described below (or analogous process to those described herein), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

The term “solvate” means a physical association of a compound of this disclosure with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

As used herein, “polymorph(s)” refer to crystalline form(s) having the same chemical structure/composition but different spatial arrangements of the molecules and/or ions forming the crystals. Compounds of the present disclosure can be provided as amorphous solids or crystalline solids. Lyophilization can be employed to provide the compounds of the present disclosure as a solid.

The term “hearing loss” refers to a sudden or gradual decrease in how well a subject can hear.

The term “balance disorder” refers to disruption in the labyrinth (the inner ear organ) that controls the balance system, which allows a subject to know where his/her body is in the environment. Such disruption generally causes the subject to feel unsteady and/or dizzy.

The term “partial or complete hearing loss” refers to different degree of a decrease in the ability to perceive sounds.

The term “acquired hearing loss” refers to loss of hearing that occurs or develops some time during the lifespan but is not present at birth.

The term “sensorineural hearing loss” refers to hearing loss caused by damage to the sensory cells and/or nerve fibers of the inner ear.

As used herein, the term “patient” encompasses all mammalian species.

As used herein, the term “subject” refers to an animal Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. Exemplary subjects include human beings of any age with risk factors for cancer disease.

As used herein, a subject is “in need of” a treatment if such subject would benefit biologically, medically or in quality of life from such treatment (preferably, a human).

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat’, “treating” or “treatment” of any disease/disorder refers the treatment of the disease/disorder in a mammal, particularly in a human, and include: (a) ameliorating the disease/disorder, (i.e., slowing or arresting or reducing the development of the disease/disorder, or at least one of the clinical symptoms thereof); (b) relieving or modulating the disease/disorder, (i.e., causing regression of the disease/disorder), either physically, (e.g., stabilization of a discernible symptom), physiologically, (e.g., stabilization of a physical parameter), or both); (c) alleviating or ameliorating at least one physical parameter including those which may not be discernible by the subject; and/or (d) preventing or delaying the onset or development or progression of the disease or disorder from occurring in a mammal, in particular, when such mammal is predisposed to the disease or disorder but has not yet been diagnosed as having it.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to at least partially alleviate, inhibit, prevent and/or ameliorate hearing loss and/or balance disorder.

The effective amount can vary depending on such factors as the size and weight of the subject, the type of illness, or the particular compound of the present disclosure. One of ordinary skill in the art would be able to study the factors contained herein and make the determination regarding the effective amount of the compounds of the present disclosure without undue experimentation.

The regimen of administration can affect what constitutes an effective amount. The compound of the present disclosure can be administered to the subject either prior to or after the onset of hearing loss and/or balance disorder. Further, several divided dosages, as well as staggered dosages, can be administered daily or sequentially, or the dose can be continuously infused, or can be a bolus injection. Further, the dosages of the compound(s) of the present disclosure can be proportionally increased or decreased as indicated by the exigencies of the therapeutic or prophylactic situation.

### Preparation of Compounds

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd}-ed., Wiley-VCH Weinheim, Germany (1999), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

The following abbreviations used herein below have the corresponding meanings:

| | |
|---|---|
| CDI | di(1H-imidazol-1-yl)methanone |
| CH₃CN/MeCN | acetonitrile |
| CH₃MgBr | methyl magnesium bromide |
| CH₃NH₂ | methanamine |
| (COCl)₂ | oxalyl dichloride |
| (COOEt)₂ | diethyl oxalate |
| CuI | copper(I) iodate |
| DCM/CH₂Cl₂ | dichloromethane |
| DIAD | diisopropyl azodiformate |
| DIEA/DIPEA | N-ethyl-N-isopropylpropan-2-aminc |
| DMF | dimethylformamide |
| DMP | Dess-Martin periodinane |
| DMSO | dimethylsulfoxide |
| EDCI | 1-(3-dimethylaminopropyl)-3-ethylcarbodiimide |
| | hydrochloride |
| Et₃N | triethylamine |
| EtOAc | ethyl acetate |
| EtOH | ethanol |
| H₂ | hydrogen |
| H₂O | water |
| HAUT | 2-(7-aza-1H-benzotriazole-1-yl)-1,1,3,3- |
| | tetramethyluronium hexafluorophosphate |
| HCl | hydrochloric acid |
| HOAc | acetic acid |
| HOBt | 1-Hydroxybenzotriazole |
| HPLC | high performance liquid chromatography |
| K₂CO₃ | potassium carbonate |
| KI | Potassium iodide |
| LiOH•H₂O | lithium hydroxide hydrate |
| m-CPBA | 3-chloroperoxybenzoic acid |
| Me₃Al | trimethylaluminium |
| MeOH | methanol |
| MgSO₄ | magnesium sulphate |
| mL | millilitre |
| MS | mass spectrometer |
| MsCl | methanesulfonyl chloride |
| N₂ | nitrogen |
| NaBH₃CN | sodium cyanoborohydride |
| NaB(OAc)₃H | sodium triacetoxyhydroborate |
| NaHCO₃ | sodium bicarbonate |
| Na₂SO₄ | sodium sulfate |
| Na₂SO₃ | sodium sulfite |
| NH₃•H₂O/NH₄OH | ammonia |
| NH₂OH•HCl | hydroxylamine hydrochloride |
| NBS | N-Bromosuccinimide |
| Pd(OH)₂/C | palladium hydroxide on carbon |
| PPh₃ | triphenylphosphine |
| rt | room temperature |
| t-BuOK | potassium tert-butoxide |
| TFA | trifluoroacetic acid |
| THF | tetrahydrofuran |

### LC/MS Methods Employed in Characterization of Examples

LC/MS data were recorded using Agilent 1100 HPLC systems with Waters Micromass ZQ, or Waters ACQUITY UPLC with Waters SQ detector or with Waters ACQUITY QDa detector.

### NMR Employed in Characterization of Examples

NMR spectra were obtained with Bruker Fourier transform spectrometers operating at frequencies as follows: ¹H NMR: 400 MHz (Bruker). ¹³C NMR: 100 MHz (Bruker). Spectra data are reported in the format: chemical shift (multiplicity, number of hydrogens). Chemical shifts are specified in ppm downfield of a tetramethylsilane internal standard (8 units, tetramethylsilane=0 ppm) and/or referenced to solvent peaks, which in ¹H NMR spectra appear at 2.50 ppm for CD₃SOCD₃, 3.31 ppm for CD₃OD, 1.94 for CD₃CN, 4.79 for D₂O, 5.32 for CD₂Cl₂, and 7.26 ppm for CDCl₃, and which in ¹³C NMR spectra appear at 39.7 ppm for CD₃SOCD₃, 49.0 ppm for CD₃OD, 1.32 and/or 118.26 for CD₃CN, 53.84 for CD₂Cl₂, and 77.0 ppm for CDCl₃. All ¹³C NMR spectra were proton decoupled.

### Methods Employed in the Purification of the Examples

Purification of intermediates and final products was carried out via either normal or reverse phase chromatography. Normal phase chromatography was carried out using prepacked SiO₂ cartridges (e.g., RediSep® Rf columns from Teledyne Isco, Inc.) eluting with gradients of appropriate solvent systems (e.g., hexanes and ethyl acetate; DCM and MeOH; or unless otherwise indicated). Reverse phase preparative HPLC was carried out using the methods described in individual example experimental procedure with corresponding information on colume, basic/neutral/acidic condition, and acetonitrile gradient range.

### General Synthetic Schemes

Schemes 1-3 (shown below) describe potential routes for preparing the compounds of the present disclosure which include compounds of Formula (I) and subformulae thereof. The starting materials for the below reaction scheme are commercially available or can be prepared according to methods known to one skilled in the art or by methods disclosed herein. Compounds of Formula (I) can be made substantially optically pure by either using substantially optically pure starting material or by separation chromatography, recrystallization or other separation techniques well-known in the art. For a more detailed description, see the Example section below.

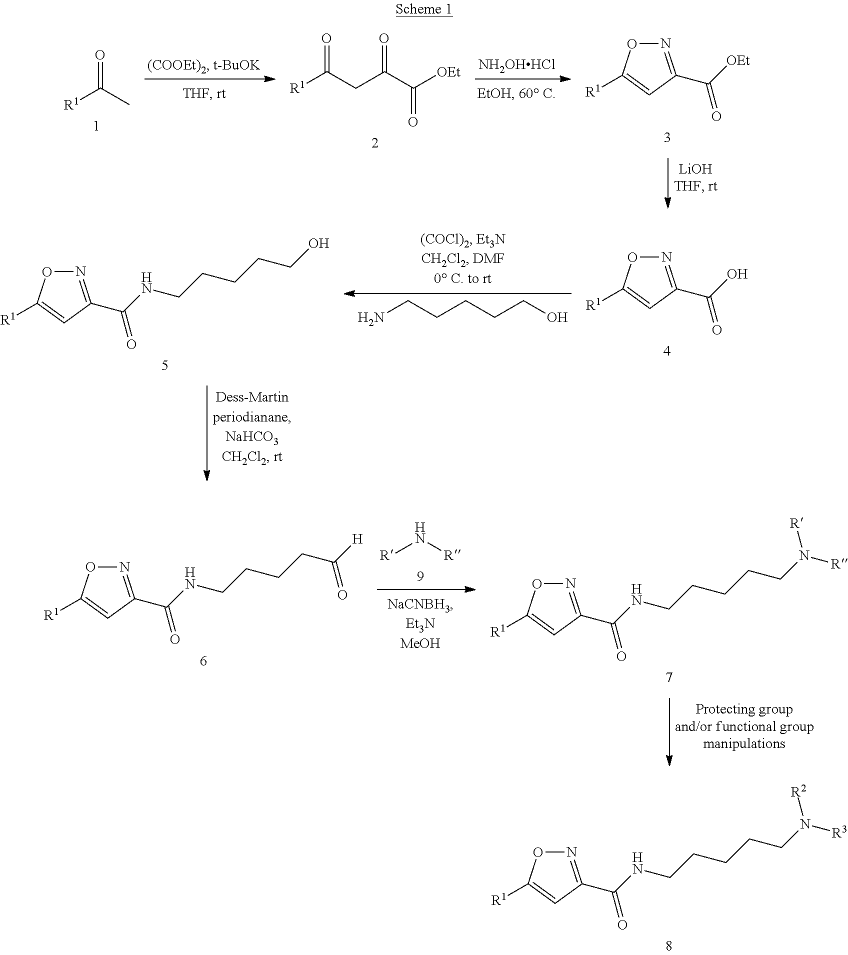

As depicted in scheme 1, aromatic methyl ketone 1 is treated with strong base (such as t-BuOK) and diethyl oxalate to yield α-ketyl ester 2, which cyclizes with hydroxylamine hydrochloride to give isoxazole ester 3. Subsequent hydrolysis of compound 3 by LiOH furnishes acid 4, which is converted to the corresponding acid chloride via oxalyl chloride and then couples with 5-aminopentan-1-ol to generate amide 5. The alcohol of compound 5 is further oxidized by Dess-Martin periodinane to give aldehyde 6, which undergoes reductive amination with various amine 9 (R′ and R″ each represent various substitutents on the N of the amine 9) in the presence of NaCNBH₃ or NaBH(OAc)₃ to generate corresponding tertiary amine 7. Depending on the structure of amine 9, compound 7 can go through protecting group and/or functional group manipulations to provide target molecule 8.

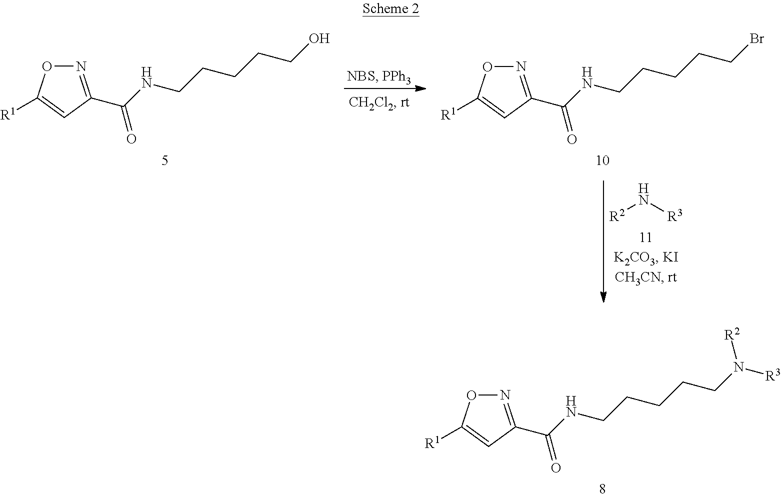

Alternatively in Scheme 2, alcohol 5 is converted to the corresponding bromide 10 via NBS, which undergoes alkylation with various amines 11 in the presence of weak base (such as K₂CO₃) to provide the target molecule 8.

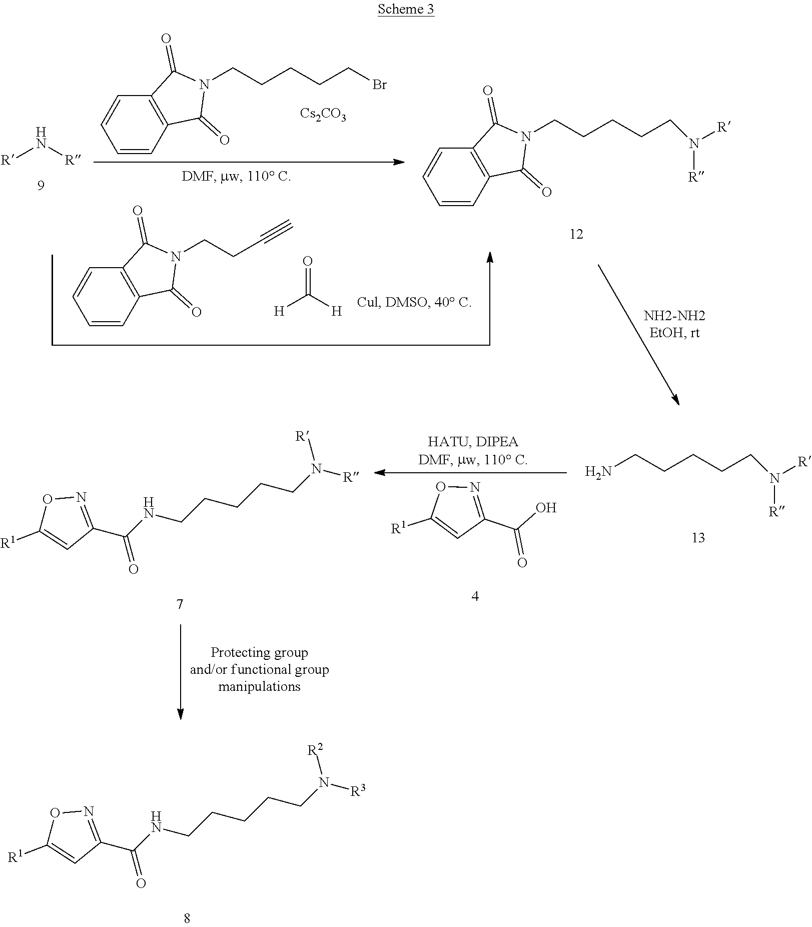

In addition, as shown in Scheme 3, secondary amine 9 (R′ and R″ each represent various substitutents on the N of the amine 9) either undergoes alkylation in the presence of base (such as Cs₂CO₃) with 2-(5-bromopentyl)isoindoline-1,3-dione, or goes through three component coupling reaction with 2-(but-3-yn-1-yl)isoindoline-1,3-dione and formaldehyde in the presence of catalytic copper iodide to form tertiary amine 12. Compound 12 is de-protected with hydrazine to provide primary amine 13, which then reacts with acid 4 under general amide coupling conditions (such as HATU, EDCI/HOBt, etc.) to provide tertiary amine 7. Depending on the structure of amine 9, compound 7 can go through protecting group and/or functional group manipulations to provide target molecule 8.

### Pharmaceutical Compositions and Combinations

The compounds of the present disclosure are typically used as a pharmaceutical composition (e.g., a compound of the present disclosure and at least one pharmaceutically acceptable carrier). A “pharmaceutically acceptable carrier (diluent or excipient)” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

In one aspect, the present disclosure provides a pharmaceutical composition comprising a compound of the present disclosure, or a pharmaceutically acceptable salt thereof, and a pharmaceutically acceptable carrier. In a further embodiment, the composition comprises at least two pharmaceutically acceptable carriers, such as those described herein. For purposes of the present disclosure, unless designated otherwise, solvates and hydrates are generally considered compositions. Preferably, pharmaceutically acceptable carriers are sterile. The pharmaceutical composition can be formulated for particular routes of administration such as oral administration, parenteral administration, and rectal administration, etc. In addition, the pharmaceutical compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Tablets may be either film coated or enteric coated according to methods known in the art.

Suitable compositions for oral administration include an effective amount of a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present disclosure further provides anhydrous pharmaceutical compositions and dosage forms comprising the compounds of the present disclosure as active ingredients, since water may facilitate the degradation of certain compounds.

Anhydrous pharmaceutical compositions and dosage forms of the disclosure can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The present disclosure further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compound of the present disclosure is typically formulated into pharmaceutical dosage forms to provide an easily controllable dosage of the drug and to give the patient an elegant and easily handleable product. The dosage regimen for the compounds of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. Compounds of this disclosure may be administered in a single daily dose, or the total daily dosage may be administered in divided doses of two, three, or four times daily.

The present disclosure further provides pharmaceutical compositions which can be delivered locally to the subject, including administration in the form of solid, semi-solid, liquid, gels, and microspheres, etc., into the outer ear, middle ear or inner ear. Compositions of the present disclosure can be administered by a number of methods sufficient to deliver the composition to the inner ear. Such methods include, but are not limited to, auricular administration (e.g., by transtympanic wicks or catheters), intraauricular administration, intratympanic administration, intracochlear administration, intravestibular administration and intralabyrinth administration.

As used herein, the term “auricular administration” refers to a method of using a catheter or wick device to administer a composition across the tympanic membrane to the inner ear of the subject. To facilitate insertion of the wick or catheter, the tympanic membrane may be pierced using a suitably sized syringe. The devices could also be inserted using any other methods known to those of skill in the art, e.g., surgical implantation of the device. In particular embodiments, the wick or catheter device may be a stand alone device, meaning that it is inserted into the ear of the subject and then the composition is controllably released to the inner ear. In other particular embodiments, the wick or catheter device may be attached or coupled to a pump or other device that allows for the administration of additional compositions. The pump may be automatically programmed to deliver dosage units or may be controlled by the subject or medical professional.

As used herein, the term “Intraauricular” administration refers to administration of a composition to the outer, the middle or inner ear of a subject by directly injecting the composition. “intratympanic” administration refers to the injection or perfusion of a composition across the tympanic membrane into the middle ear, such that the composition may diffuse across the round window membrane into the inner ear. “Intracochlear” administration refers to direct delivery of a composition into the cochlea. “Intravestibular” administration refers to direct delivery of a composition into the vestibular organs “Intralabyrinth” administration refers to direct delivery of a composition into the inner ear fluid compartment to expose the inner ear including the semicircular canals, the vestibule and cochlea to the composition.

In one embodiment, a syringe and needle apparatus is used to administer compositions to a subject using auricular administration. A suitably sized needle is used to pierce the tympanic membrane and a wick or catheter comprising the composition is inserted through the pierced tympanic membrane and into the middle ear of the subject. The device may be inserted such that it is in contact with the round window or immediately adjacent to the round window. Exemplary devices used for auricular administration include, but are not limited to, transtympanic wicks, transtympanic catheters, transtympanic pumps, round window microcatheters (small catheters that deliver medicine to the round window), and Silverstein Microwicks™ (small tube with a “wick” through the tube to the round window, allowing regulation by subject or medical professional).

In another embodiment, a syringe and needle apparatus is used to administer compositions to a subject into the middle and/or inner ear. The formulation may be administered directly onto the round window membrane via intratympanic injection, or may be administered directly to the cochlea via intracochlear injection, or directly to the vestibular organs via intravestibular injection, or directly to the semicircular canals, the vestibule and the cochlea via intralabyrinth injection.

In still another embodiment, the delivery device can be an apparatus designed for administration of compositions to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. Patent Application Publication 2007/0167918, which is incorporated herein by reference for such disclosure, further describes a combined otic aspirator and medication dispenser for transtympanic fluid sampling and medicament application.

In one embodiment, the compositions may be locally administered to the subject. In another embodiment, the compositions may be administered to the subject by auricular administration. In still another embodiment, the compositions may be administered to the subject by intraauricular administration. In still another embodiment, the compositions may be administered to the subject by intratympanic administration. In still another embodiment, the compositions may be administered to the subject by intracochlear administration. In still another embodiment, the compositions may be administered to the subject by intravestibular administration. In still another embodiment, the compositions may be administered to the subject by intralabyrinth administration.

In one embodiment, the compositions comprise one or more components that enhance the availability of the active ingredients of the composition to the cochlea, and/or provide extended or immediate release of active ingredients of the composition to the inner ear. In one embodiment, the one or more components are pharmaceutically acceptable carriers.

In another embodiment, the compositions comprise one or more pharmaceutically acceptable carriers that will facilitate the delivery of the composition across biological barriers that separate the middle and inner ear, e.g., the round window, thereby efficiently delivery a therapeutically effective amount of the composition to the inner ear. Efficient delivery to the cochlea, Organ of Corti, vestibular organs, and/or the inner ear perilymph or endolymph fluid space is desired because these tissues/organs host the supporting cells that promote sensory hair cell regeneration when treated or contacted with compositions of the present disclosure.

Intratympanic delivery to the inner ear can be performed via the injection or perfusion of the composition to the middle ear with the aim of the composition diffusion through the round window membrane into the inner ear. Delivery systems suitable for the intratympanic administration are well known and can be found in, for example, Liu et al., Acta Pharmaceutica Sinica B 2013; 3(2):86-96; Kechai et al., International Journal of Pharmaceutics 2015; 494: 83-101; and Ayoob et al., Expert Opinion on Drug Delivery, 2015; 12(3): 465-479.

In certain instances, it may be advantageous to administer the compound of the present disclosure in combination with one or more therapeutically active agents, for example, those therapeutically active agents related to relevant hair cell development/regeneration pathways, including but not limited to, Notch sigaling, FGF signaling, Wnt Signaling, Shh signaling, cell cycle/stem cell aging, miRNA and epigenetic regulations.

The term “combination therapy” refers to the administration of two or more therapeutic agents to treat a therapeutic disease, disorder or condition described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. The compound of the present disclosure and additional therapeutic agents can be administered via the same administration route or via different administration routes. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the diseases, conditions or disorders described herein.

In one embodiment, the present disclosure provides pharmaceutical compositions comprising at least one compound of the present disclosure or a pharmaceutically acceptable salt thereof together with a pharmaceutically acceptable carrier suitable for administration to a human or animal subject, either alone or together with one or more other therapeutically active agents related to those relevant hair cell development/regeneration pathways as described in the above.

In another embodiment, the present disclosure provides methods of treating a human or animal subject for hearing loss or balance disorder, comprising administering to the subject a therapeutically effective amount of a compound of the present disclosure or a pharmaceutically acceptable salt thereof, either alone or in combination with one or more other therapeutically active agents related to those relevant hair cell development/regeneration pathways as described in the above.

In particular, compositions will either be formulated together as a combination therapeutic or administered separately.

In combination therapy for treatment of hearing loss or balance disorder, the compound of the present disclosure and other therapeutically active agent(s) may be administered simultaneously, concurrently or sequentially with no specific time limits, wherein such administration provides therapeutically effective levels of the two compounds in the body of the subject.

In a preferred embodiment, the compound of the present disclosure and the other therapeutically active agent(s) is generally administered sequentially in any order by infusion, orally or locally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present disclosure and other therapeutically active agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present disclosure, a kit comprising two or more separate pharmaceutical compositions, at least one of which contains a compound of the present disclosure is provided. In one embodiment, the kit comprises means for separately retaining said compositions, such as a container, divided bottle, or divided foil packet. An example of such a kit is a blister pack, as typically used for the packaging of tablets, capsules and the like.

The kit of the present disclosure may be used for administering different dosage forms, for example, oral and parenteral, for administering the separate compositions at different dosage intervals, or for titrating the separate compositions against one another. To assist compliance, the kit of the present disclosure typically comprises directions for administration.

In the combination therapies of the present disclosure, the compound of the present disclosure and the other therapeutic agent may be manufactured and/or formulated by the same or different manufacturers. Moreover, the compound of the present disclosure and the other therapeutic (or pharmaceutical agent) may be brought together into a combination therapy: (i) prior to release of the combination product to physicians (e.g. in the case of a kit comprising the compound of the present disclosure and the other therapeutic agent); (ii) by the physician themselves (or under the guidance of the physician) shortly before administration; (iii) in the patient themselves, e.g. during sequential administration of the compound of the present disclosure and the other therapeutic agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition or combination of the present disclosure can be in unit dosage of about 1-10000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties may be demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg

### Pharmacology and Utility

The present disclosure relates generally to compounds, compositions and methods for treating hearing loss and balance disorder associated with the damage or loss of sensory hair cells in the inner ear by increasing, promoting, stimulating or inducing the regeneration of sensory hair cells in the inner ear. Therefore, a brief review of the anatomy of the ear may be helpful in understanding the present disclosure.

The anatomy of the ear is well known to those of ordinary skill in the art (see, e.g., Gray's Anatomy, Revised American Edition (1977), pages 859-867). The ear is generally divided into three portions: the outer ear, middle ear, and inner ear. The outer ear is composed of auricle (the pinna), the auditory canal, and the outward facing portion of the tympanic membrane (ear drum). The function of the outer ear, in part, is to collect and direct sound waves through the auditory canal towards the tympanic membrane and the middle ear.

The middle ear is an air-filled cavity that includes the tympanic cavity, three ear bones (auditory ossicles): the malleus, the incus and the stapes, oval window and round window, which connects the middle ear with the inner ear. The auditory ossicles are arranged to provide a mechanical linkage between the tympanic membrane and the oval window to the fluid-filled inner ear, where sound is transformed and transduced to the inner ear for further processing.

The inner ear contains sensory organs for hearing and balance. The cochlea senses sound; the balance organ includes semicircular canals, which sense angular acceleration; and the otolithic organs (utricle and saccule), which sense linear acceleration. The round window that connects the cochlea to the middle ear. In each of these sensory portions, specialized sensory hair cells are arrayed upon one or more layers of inner ear supporting cells. Supporting cells underlie, at least partially surround, and physically support sensory hair cells within the inner ear. The stereocilia on the sensory hair cells are physically deflected in response to sound or motion, and their deflection is transmitted to nerves which send nerve impulses to the brain for processing and interpretation.

In particular, the cochlea includes the Organ of Corti which is primarily responsible for sensing sound. The Organ of Corti includes a basilar membrane upon which are located a variety of supporting cells, including border cells, inner pillar cells, outer pillar cells, inner phalangeal cells, Dieter's cells and Hensen's cells. Supporting cells surround and seperate inner hair cells and outer hair cells. The tectorial membrane is disposed above inner hair cells and outer hair cells.

Hearing loss and balance disorders are mainly caused by damage or loss of the sensory hair cells in the cochlea. In mammals, loss or damage to sensory hair cells results in permanent hearing loss or balance disorders, because they are generated only during embryonic development and do not spontaneously regenerate upon damage or cell loss during one's life time. It is widely accepted that although cells capable of generating sensory hair cells are present in the inner ear, natural sensory hair cell regeneration in the inner ear is low (Li et al., Trends Mol. Med., 10, 309-315 (2004); Li et al., Nat. Med., 9, 1293-1299 (2003); Rask-Andersen et al., Hear. Res., 203, 180-191 (2005)). As a result, lost or damaged sensory hair cells may not be adequately replaced by natural physiological processes (e.g., cell differentiation) and a loss of hair cells occurs. In many individuals, such sensory hair cell loss can result in, e.g., sensorineural hearing loss and balance disorders. Therefore, therapeutic strategies that increase the number of sensory hair cells in the inner ear will benefit a patient with sensory hair cell loss or damage.

Sensory hair cell fate determination in the inner ear is controlled by specific genes and pathways. Atonal protein homologue 1 (Atoh1 or atonal) is the master regulator of inner ear hair cell development and regeneration. The importance of Atoh1 in hair cell genesis is well documented. For example, Mathl (Atoh1 homolog in mouse) is required for hair cell development and the differentiation of inner ear progenitor cells to inner ear support cells and/or sensory hair cells (Bermingham et al., Science, 284:1837-1841, 1999). In addition, adenovirus mediated Mathl overexpression in the endolymph of the mature guinea pig results in the differentiation of non-sensory cells in the mature cochlea into immature hair cells (Kawamoto et al., J. Neurosci., 23:4395-4400, 2003). The implications of these studies are twofold. First, they demonstrate that non-sensory cells of the mature cochlear retain the ability to differentiate into sensory cells, e.g., sensory hair cells. Second, they demonstrate that Mathl overexpression is necessary and sufficient to direct supporting cells transdifferentiation into hair cells. A later study furthered these findings by demonstrating that adenovirus mediated Atoh1 overexpression induces sensory hair cell regeneration and substantially improves hearing thresholds in an experimentally deafened animal model (Izumikawa et al., Nat. Med., 11:271-276, 2005).

This suggests that although the mammalian cochlear sensory epithelium has lost the ability to spontaneously regenerate, the molecular activity required for inducing hair cell fate is still present and functional in mature supporting cells. These findings also suggest that activation of endogenous Atoh1 expression by pharmacological intervention could be an effective approach to stimulate sensory hair cell regeneration for treating hearing loss and balance disorders.

The present disclosure provides compounds, compositions and methods which are capable of increasing Atoh1 expression and/or activity in a subject. The present disclosure also provides compounds, compositions and methods which can increase or promote sensory hair cell regeneration. The present disclosure also provides compounds, compositions and methods which can increase the number of sensory hair cells in the inner ear of the subject. Consequently, the compounds, compositions and methods described herein can be used to treat hearing loss and/or balance disorders that result from the damage or loss of sensory hair cells in a subject.

The compounds of present disclosure in free form or in pharmaceutically acceptable salt form, exhibit valuable pharmacological properties, which can be demonstrated at least by using any one of the following test procedures. Compounds of the present disclosure were assessed for their ability to increase the Atoh1 expression in mouse cerebellar neural precursor cells. The ability of compounds of the present disclosure to induce new hair cell formation was assessed in ex vivo hair cell induction assay using 6-thy-postnatal mouse cochlea explants with hair cell damage.

### EXAMPLES

The following Examples have been prepared, isolated and characterized using the methods disclosed herein. The following examples demonstrate a partial scope of the disclosure and are not meant to be limiting of the scope of the disclosure.

Unless specified otherwise, starting materials are generally available from a non-limiting commercial sources such as TCI Fine Chemicals (Japan), Shanghai Chemhere Co., Ltd. (Shanghai, China), Aurora Fine Chemicals LLC (San Diego, Calif.), FCH Group (Ukraine), Aldrich Chemicals Co. (Milwaukee, Wis.), Lancaster Synthesis, Inc. (Windham, N H), Acros Organics (Fairlawn, N.J.), Maybridge Chemical Company, Ltd. (Cornwall, England), Tyger Scientific (Princeton, N.J.), AstraZeneca Pharmaceuticals (London, England), Chembridge Corporation (USA), Matrix Scientific (USA), Confer Chem & Pharm Co., Ltd (China), Enamine Ltd (Ukraine), Combi-Blocks, Inc. (San Diego, USA), Oakwood Products, Inc. (USA), Apollo Scientific Ltd. (UK), Allichem LLC. (USA) and Ukrorgsyntez Ltd (Latvia).

### INTERMEDIATES

### Intermediate A: 5-(Thiophen-2-yl)isoxazole-3-carboxylic acid

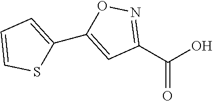 [SMILES: O=C(O)c1cc(-c2cccs2)on1]

### Step 1: Ethyl 2,4-dioxo-4-(thiophen-2-yl)butanoate

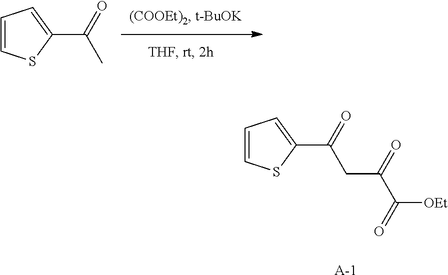

To a solution of 1-(thiophen-2-yl)ethan-1-one (50 g, 396.2 mmol, 1.0 eq) and (COOEt)₂ (72.39 g, 495.3 mmol, 1.25 eq) in anhydrous THF (2.0 L) was added t-BuOK (57.8 g, 515.1 mmol, 1.3 eq) in small portions at 15-25° C. Then the mixture was stirred at rt for 2 hours. The mixture was poured into water (800 mL), acidified to pH 2 with 1N HCl, and then the mixture was extracted with ethyl acetate (3*500 mL). The organic layer was separated and washed with brine (1 L), dried over anhydrous sodium sulfate, and concentrated to give the crude title product (100 g) as a yellow solid which was used without further purification.

### Step 2: Ethyl 5-(thiophen-2-yl)isoxazole-3-carboxylate

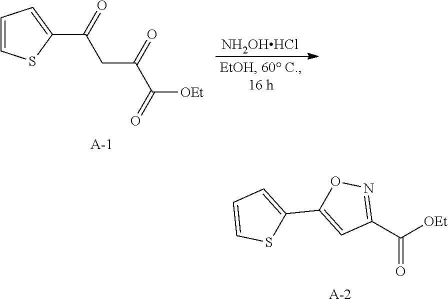

To a solution of compound A-1 (89 g, 393.3 mmol, 1.0 eq) in anhydrous ethanol (2 L) was added compound NH₂OH.HCl (54.64 g, 786.7 mmol, 2 eq). The mixture was stirred at 60° C. for 16 hours. The reaction mixture was concentrated. Water (200 mL) was added and the mixture was extracted with EtOAc (3*200 mL). The organic layer was concentrated under the vacuum to afford the crude title product (90 g) which was used without further purification.

### Step 3: 5-(Thiophen-2-yl)isoxazole-3-carboxylic acid

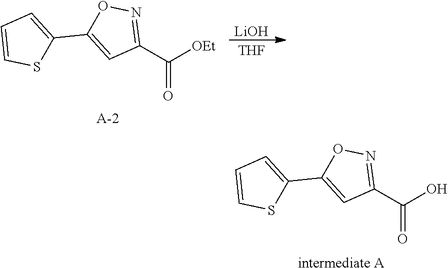

To a solution of compound A-2 (80 g, 358.3 mmol, 1.0 eq) in THF (200 mL) was added a solution of LiOH.H₂O (17.16 g, 716.6 mmol, 2.0 eq) in water (358.3 mL). The resulting mixture was stirred at 15-22° C. for 2 hours. The reaction mixture was concentrated under reduced pressure to remove THF. The residue was acidified to pH 1 with 1 N HCl and extracted with EtOAc (3*300 mL). The combined organic layers were concentrated under the vacuum. The solid was triturated with EtOAc, filtered and dried to give the title compound (42.6 g, 60.9% yield) as a white solid. ¹H NMR (400 MHz, CDCl₃) δ ppm 7.60-7.59 (dd, J=3.6, 1.2 Hz, 1H), 7.54-7.52 (dd, J=4.8, 1.2 Hz, 1H), 7.18-7.16 (dd, J=4.8, 3.6 Hz, 1H), 6.84 (s, 1H).

### Intermediate B: N-(5-Oxopentyl)-5-(thiophen-2-yl)isoxazole-3-carboxamide

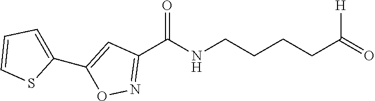 [SMILES: O=CCCCCNC(=O)c1cc(-c2cccs2)on1]

### Step 1: N-(5-Hydroxypentyl)-5-(thiophen-2-yl)isoxazole-3-carboxamide

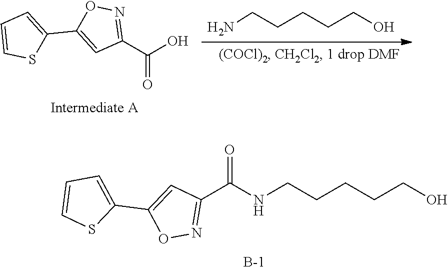

To a solution of compound Intermediate A (10 g, 51.23 mmol, 1.0 eq) in anhydrous CH₂Cl₂ (100 mL) was added (COCl)₂ (19.5 g 13.1 mL, 153.6 mmol, 3.0 eq) dropwise under N₂ protection, then one drop DMF was added at 0° C. The mixture was stirred at rt for 2 hours. Then the mixture was concentrated under the vacuum and the residue was diluted with CH₂Cl₂ (50 mL), then the mixture was added to a solution of 5-aminopentan-1-ol (7.93 g, 76.85 mmol, 1.5 eq) and Et₃N (15.5 g, 153.69 mmol, 3.0 eq) in CH₂Cl₂ (100 mL) dropwise at 0° C. The resulted mixture was stirred at rt for 1 hour. Then the reaction was quenched with water (50 mL) and extracted with CH₂Cl₂ (3*50 mL). The organic layers were dried over anhydrous Na₂SO₄, filtered and concentrated under the vacuum to afford the title compound (12.5 g, 87.03% yield) as a white solid. MS (ESI) m/z 302.9 [M+Na]⁺.

### Step 2: N-(5-Oxopentyl)-5-(thiophen-2-yl)isoxazole-3-carboxamide

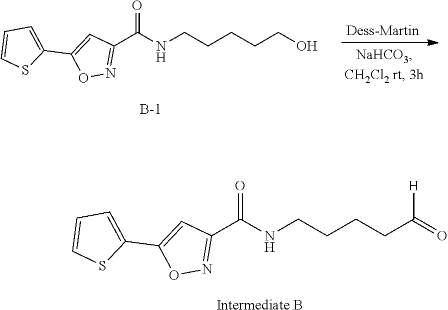

To a solution of compound B-1 (10 g, 35.67 mmol, 1.0 eq) in CH₂Cl₂ (200 mL) was added NaHCO₃ (13.48 g, 160.5 mmol, 4.5 eq), followed by DMP (22.69 g, 53.5 mmol, 1.5 eq). The resulting mixture was stirred at rt for 3 hours. The mixture was slowly poured into saturated NaHCO₃ aqueous solution (100 mL) and extracted with CH₂Cl₂ (3*100 mL). The combined organic layers were dried over anhydrous sodium sulfate, filtered and concentrated in vacuum and the residue was purified by silica gel chromatography eluting with petroleum/EtOAc from 100/0 to 1/1 to give the title compound (4.5 g, 45.3% yield) as a white solid. MS (ESI) m/z 300.9 [M+Na]⁺.

### Intermediate C: 5-(4-Fluorophenyl)-N-(5-oxopentyl)isoxazole-3-carboxamide

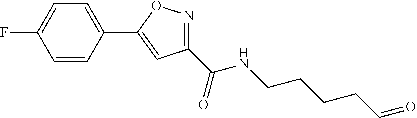 [SMILES: O=CCCCCNC(=O)c1cc(-c2ccc(F)cc2)on1]

The title compound was prepared by using a procedure similar to that of Intermediate of B by replacing of intermediate A with 5-(4-fluorophenyl)isoxazole-3-carboxylic acid (which was made using the similar method as intermediate A) in 28% yield as a white solid. MS (ESI) m/z 312.9 [M+H]⁺. ¹H NMR (400 MHz, CDCl₃) δ ppm 9.81 (t, J=1.2 Hz, 1H), 7.82-7.78 (m, 2H), 7.23-7.16 (m, 2H), 6.92 (s, 1H), 3.50 (q, J=6.4 Hz, 2H), 2.59-2.50 (m, 2H), 1.80-1.64 (m, 4H).

### Intermediate D: Azetidine-3-sulfonamide

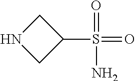

### Step 1: Benzyl 3-(acetylthio)azetidine-1-carboxylate

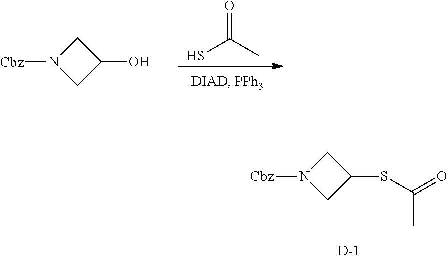

To a solution of PPh₃ (7.91 g, 30.16 mmol, 1.25 eq) in THF (30 mL) at −78° C. was added DIAD (5.95 g, 29.44 mmol, 1.22 eq) in THF (20 mL). After stirred for 10 min, thioacetic acid (2.39 g, 2.24 mL, 31.37 mmol, 1.3 eq) in THF (20 mL) was added. After additional 10 min, a solution of benzyl 3-hydroxyazetidine-1-carboxylate (5 g, 24.13 mmol, 1.0 eq) in THF (30 mL) was added. The reaction was stirred at −78° C. for 1 hour and then allowed to warm to 25° C. for 14 hours. The reaction mixture was quenched with brine (30 mL). The aqueous phase was extracted with EtOAc (3*20 mL). The combined organic phase was dried over Na₂SO₄, filtered and concentrated under reduced pressure. The residue was purified by silica gel column chromatography eluting with petroleum/EtOAc from 50/1 to 5/1 to afford the title compound (2.0 g, 31% yield) as a light yellow oil. ¹H NMR (400 MHz, CDCl₃) δ ppm 7.38-7.28 (m, 5H), 5.11 (s, 2H), 4.49-4.45 (m, 2H), 4.24-4.21 (m, 1H), 3.94-3.90 (m, 2H), 2.35 (s, 3H).

### Step 2: Benzyl 3-(chlorosulfonyl)azetidine-1-carboxylate

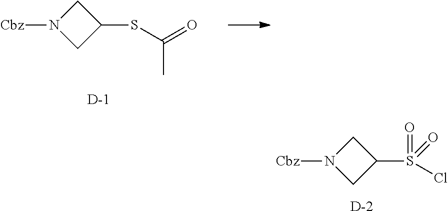

To a solution of compound D-1 (1.1 g, 4.15 mmol, 1.0 eq) in CH₂Cl₂ (20 mL) was added water (5 mL). The mixture was cooled to 0° C. and chlorine gas was bubbled through at 0-5° C. with stirring for 1 hour. The layers were separated and the DCM layer containing compound D-2 (4.15 mmol) was used directly in the next step.

### Step 3: Benzyl 3-sulfamoylazetidine-1-carboxylate

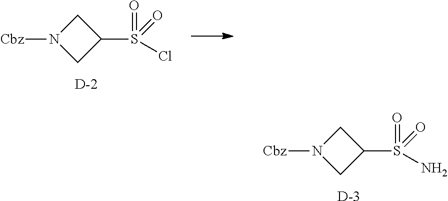

To a solution of NH₃.H₂O (40 mL, 0.34 mol, 28% wt, 82.7 eq) was added a solution of compound D-2 (4.15 mmol, 1.0 eq) in CH₂Cl₂ (20 mL) at 0-5° C. The mixture was stirred at 26° C. for 14 hours. The aqueous phase was extracted with CH₂Cl₂ (2*40 mL). The combined organic phase was dried over Na₂SO₄, filtered, concentrated. The residue was purified by acidic preparative HPLC (Boston Green ODS 150*30 5 u, gradient: 22-32% B (A=0.1% TFA/water), B═CH₃CN), flow rate: 30 mL/min) to afford the title compound (0.35 g, 31.2% yield) as a light yellow solid. MS (ESI) m/z 292.9 [M+23]⁺. ¹H NMR (400 MHz, CDCl₃) δ ppm 7.36-7.31 (m, 5H), 5.13 (s, 2H), 5.10 (s, 2H), 4.32-4.22 (m, 4H), 4.02-4.00 (m, 1H).

### Step 4: Azetidine-3-sulfonamide

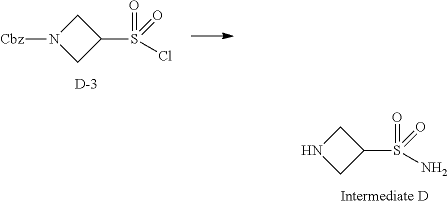

To a solution of compound D-3 (0.35 g, 1.29 mmol, 1.0 eq) in MeOH (3 mL) was added Pd/C (0.1 g, 10% wt). The mixture was stirred at 25° C. under hydrogen atmosphere (15 psi) for 4 hours. The mixture was filtered, and the cake was washed with MeOH (2*5 mL). The filtrate was concentrated to give the title compound (160 mg, 90.7% yield) as a light yellow solid. MS (ESI) m/z 136.9 [M+1]⁺. ¹H NMR (400 MHz, DMSO-d₆) δ ppm 6.90 (brs, 2H), 4.10-4.04 (m, 1H), 3.74-3.70 (m, 2H), 3.60-3.56 (m, 2H).

### Example 1

### N-(5-((3S,4S)-4-carbamoyl-3-cyanopiperidin-1-yl)pentyl)-5-(thiophen-2-yl)isoxazole-3-carboxamide

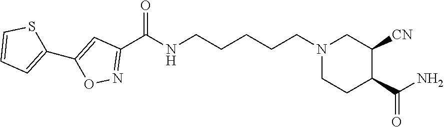 [SMILES: N#C[C@@H]1CN(CCCCCNC(=O)c2cc(-c3cccs3)on2)CC[C@@H]1C(N)=O]

### Step 1: methyl 1-benzyl-3-hydroxypiperidine-4-carboxylate

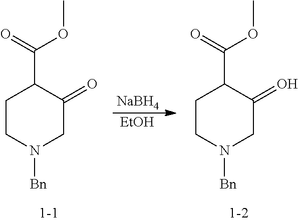 [SMILES: "EtOH" ; COC(=O)C1CCN(Cc2ccccc2)CC1=O ; "NaBH4" ; COC(=O)C1CCN(Cc2ccccc2)CC1=O]

To a mixture of compound 1-1 (3.7 g, 14.96 mmol, 1.0eq) in EtOH (60 mL) was added NaBH₄ (1.13 g, 29.92 mmol, 2.0eq) portion-wise at 0° C. The mixture was stirred for 2 hours at 0° C. The mixture was concentrated under reduced pressure. To the mixture was added water (300 mL) and EA (300 mL). The EA layer was separated and washed with brine (100 mL), dried over Na₂SO₄ and concentrated under reduced pressure to give the title compound (3.8 g, crude) as a yellow oil.

### Step 2: methyl 1-benzyl-3-((methylsulfonyl)oxy)piperidine-4-carboxylate

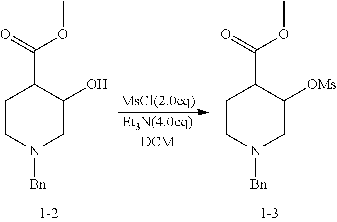 [SMILES: COC(=O)C1CCN(Cc2ccccc2)CC1O ; "Et3N(4.0eq)DCM" ; "MsCl(2.0eq)" ; COC(=O)C1CCN(Cc2ccccc2)CC1OS(C)(=O)=O]

To a mixture of compound 2-2 (1 g, 4.01 mmol, 1.0eq) in DCM (20 mL) was added MsCl (918.96 mg, 8.02 mmol, 2.0eq) portion-wise at 0° C. The mixture was allowed to warm to 15° C. and stirred for 16 h. To water (200 mL) was added the mixture. The result mixture was extracted with DCM (200 mL). The DCM layer was washed with brine, dried over Na₂SO₄ and concentrated under reduced pressure to give the title compound (1.5 g, crude) as a yellow oil, which was confirmed by LCMS. LC-MS: [M+H]⁺=328.3.

### Step 3: methyl 1-benzyl-3-cyanopiperidine-4-carboxylate

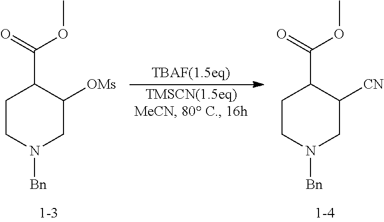 [SMILES: COC(=O)C1CCN(Cc2ccccc2)CC1OS(C)(=O)=O ; "TBAF(1.5eq)" ; "TMSCN(1.5eq)MeCN, 80° C., 16h" ; COC(=O)C1CCN(Cc2ccccc2)CC1C#N]

A mixture of compound 1-3 (1.5 g, 4.58 mmol, 1.0eq), TMSCN (681.79 mg, 6.87 mmol, 1.5eq) and TBAF (6.87 mL, 1M) in MeCN (30 mL) was stirred for 16 hat 80° C. The mixture was concentrated under reduced pressure to give a residue. The residue was purified by column chromatography on silica gel (PE:EA=20:1-10:1) to afford peak 1 (350 mg, 90%) and peak 2 (100 mg, 99% purity). Peak 1 was confirmed by LCMS(C-05663-135-P1B2) and HNMR (C-05663-135-P1A). Peak2 was confirmed by LCMS(C-05663-135-P1B3). LC-MS: [M+H]⁺=259.3. ¹H NMR (400 MHz, CDCl₃) δ ppm 7.32-7.14 (m, 5H), 3.87 (m, 0.5H), 3.67 (d, J=11.7 Hz, 3H), 3.50-3.44 (m, 1H), 3.38 (m, 0.5H), 3.04-2.91 (m, 2H), 2.87 (m, 0.5H), 2.61-2.45 (m, 1.5H), 2.42-2.17 (m, 1H), 2.16-1.92 (m, 2.5H), 1.79-1.64 (m, 0.5H).

### Step 4: 1-benzyl-3-cyanopiperidine-4-carboxamide

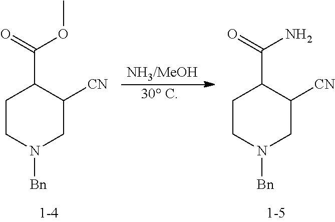 [SMILES: COC(=O)C1CCN(Cc2ccccc2)CC1C#N ; "30° C." ; "NH3/MeOH" ; N#CC1CN(Cc2ccccc2)CCC1C(N)=O]

To a mixture of compound 1-4 (peak 1, 300 mg, 1.16 mmol, 1.0eq) in MeOH (0.5 mL) were added NH₃.H2O (5 mL, 37%) at 10-15° C. The mixture was stirred for 16 h at 8-15° C. To the mixture was added EA (50 mL) and water (30 mL). The aqueous layer was extracted with EA (20 mL×2). The combined EA layers was washed with brine (50 mL), dried over Na₂SO₄ and concentrated under reduced pressure to give compound 5 (230 mg, crude) as a yellow oil. LC-MS: [M+H]⁺=244.1.

### Step 5: 3-cyanopiperidine-4-carboxamide

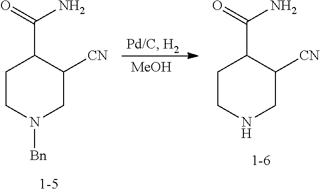 [SMILES: "MeOH" ; N#CC1CN(Cc2ccccc2)CCC1C(N)=O ; "Pd/C, H2" ; N#CC1CNCCC1C(N)=O]

To a mixture of compound 1-5 (230 mg, 0.945 mmol, 1.0eq) in MeOH (4 mL) was added Pd/C (wet, 10%, 100 mg) at 15° C. The mixture was stirred for 40 hours at 15° C. under H2 (50 psi). The mixture was filtered and the filtrate was concentrated under reduced pressure to give the title compound (70 mg, crude) as a yellow oil.

### Step 6: N-(5-((3S,4S)-4-carbamoyl-3-cyanopiperidin-1-yl)pentyl)-5-(thiophen-2-yl)isoxazole-3-carboxamide

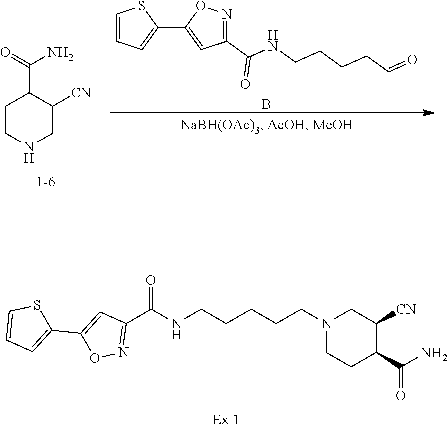

To a mixture of compound 1-6 (200 mg, crude) and Intermediate B (196.23, 705.04 μmol, 0.9eq) in MeOH (5 mL) were added HOAc (47.04 mg, 783.38 μmol, 1.0eq) and NaHB(OAc)₃ (332.06 mg, 1.57 μmol, 2.0eq) at 25° C. The mixture was stirred for 3 h at 25° C. To the mixture was added H₂O (1 mL). The mixture was filtered. The filtrate was purified by Prep-HPLC (base) and triturated with MeOH (3 mL) to give the title compound (25.91 mg, 100% purity) as a white solid. ¹H NMR (400 MHz, CD₃OD) δ ppm 7.74-7.64 (m, 2H), 7.23 (dd, J=3.8, 5.0 Hz, 1H), 6.93 (s, 1H), 3.42 (t, J=7.1 Hz, 2H), 3.22 (d, J=11.7 Hz, 1H), 3.00 (d, J=11.2 Hz, 1H), 2.56-2.35 (m, 3H), 2.23 (dd, J=2.3, 11.6 Hz, 1H), 2.09 (dt, J=3.5, 10.8 Hz, 1H), 2.03-1.86 (m, 2H), 1.76-1.53 (m, 4H), 1.52-1.38 (m, 2H). LC-MS: [M+H]⁺=416.4.

### Example 2

### N-(5-((3S,4R)-4-cyano-3-hydroxypiperidin-1-yl)pentyl)-5-(4-fluorophenyl)isoxazole-3-carboxamide

### Step 1: (3R,4S)-1-benzyl-4-cyanopiperidin-3-yl 4-nitrobenzoate

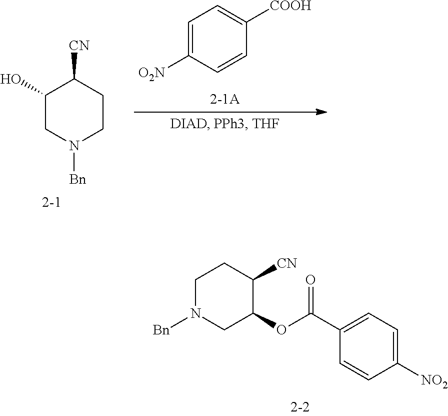 [SMILES: N#C[C@H]1CCN(Cc2ccccc2)C[C@@H]1O ; O=C(O)c1ccc([N+](=O)[O-])cc1 ; N#C[C@@H]1CCN(Cc2ccccc2)C[C@@H]1OC(=O)c1ccc([N+](=O)[O-])cc1]

To a mixture of compound 2-1 (1.1 g, 5.09 mmol, 1.0eq), compound 2-1A (849.97 mg, 5.09 mmol, 1.0eq) and PPh₃ (1.6 g, 6.10 mmol, 1.2eq) in THF (10 mL) was added DIAD (1.23 g, 6.10 mmol, 1.2eq) at 0° C. The mixture was stirred for 3 hours at 30° C. The mixture was concentrated under reduced pressure. To the mixture was added water (100 mL) and EA (100 mL). The EA layer was dried over Na2SO4 and concentrated under reduced pressure to give a residue. To the residue was added HCl (1M, 50 mL) and MTBE (50 mL). The mixture was filtered and the filter cake was concentrated under reduced pressure to give the title compound (1.3 g, crude) as a white solid. LC-MS: [M+H]⁺=366.2.

### Step 2: (3R,4S)-1-benzyl-3-hydroxypiperidine-4-carbonitrile

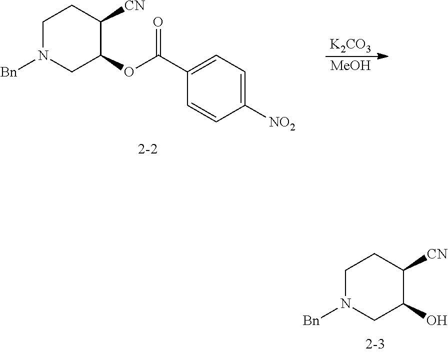 [SMILES: "MeOH" ; N#C[C@@H]1CCN(Cc2ccccc2)C[C@@H]1OC(=O)c1ccc([N+](=O)[O-])cc1 ; "K2CO3" ; N#C[C@@H]1CCN(Cc2ccccc2)C[C@@H]1O]

To a mixture of 2-2 (1 g, 2.74 mmol, 1.0 eq) in MeOH (10 mL) was added K2CO3 (756.5 mg, 5.47 mmol, 2.0 eq) (756.5 mg, 5.47 mmol, 2.0 eq) at 25° C. The mixture was stirred for 40 hs at 25° C. To water (100 mL) was added the mixture and EA (100 mL). The organic layer was washed with brine (50 mL), dried over Na₂SO₄ and concentrated under reduced pressure to give the title compound (500 mg, crude) as a yellow oil. ¹H NMR (400 MHz, DMSO-d₆) δ ppm 7.37-7.28 (m, 5H), 4.06-3.94 (m, 1H), 3.57-3.49 (m, 1H), 3.46-3.40 (m, 1H), 3.07 (mz, 1H), 2.87-2.71 (m, 2H), 2.46-2.32 (m, 1H), 2.14-1.54 (m, 3H).

### Step 3: (3R,4S)-3-hydroxypiperidine-4-carbonitrile

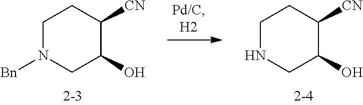 [SMILES: N#C[C@@H]1CCN(Cc2ccccc2)C[C@@H]1O ; N#C[C@@H]1CCNC[C@@H]1O]

To a mixture of compound 2-3 (200 mg, 924.73 umol, 1.0 eq) in MeOH (4 mL) was added Pd/C (wet, 100 mg, 10%) at 25° C. The mixture was stirred for 2 h at 25° C. under H2 (15 psi). The mixture was filtered and the filtrate was concentrated under reduced pressure to give the title compound (130 mg, crude) as a yellow oil.

### Step 4: N-(5-((3S,4R)-4-cyano-3-hydroxypiperidin-1-yl)pentyl)-5-(4-fluorophenyl)isoxazole-3-carboxamide

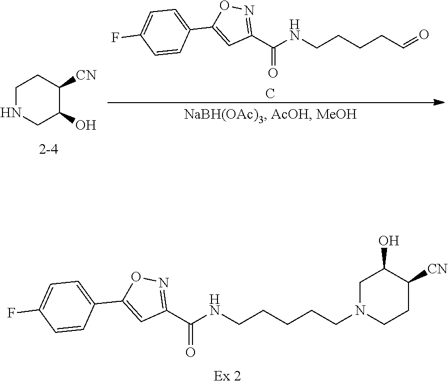

To a mixture of compound 2-4 (65 mg, crude) and Intermediate C (92.04 mg, 317.07 μmol, 0.8 eq) in MeOH (2 mL) were added HOAc (23.8 mg, 396.33 μmol, 1.0 eq) and NaHB(OAc)3 (168.0 mg, 792.67 μmol, 2.0 eq) at 25° C. The mixture was stirred for 1 h at 25° C. The mixture was filtered. The filtrate was purified by Prep-HPLC (base, then TFA), and then SFC to give two peaks both as a white solid. The title compound (Peak 1) (10.27 mg) was confirmed by LCMS, HNMR, 2D_NMR and SFC. LCMS: RT=0.950 min [M+H]⁺=401.4; ¹H NMR (400 MHz, METHANOL-d₄, C-05763-051-P1B1) δ=7.89-7.75 (m, 2H), 7.18 (t, J=8.8 Hz, 2H), 6.95 (s, 1H), 3.57-3.49 (m, 1H), 3.46-3.38 (m, 1H), 3.30 (t, J=7.1 Hz, 2H), 3.10 (ddd, J=2.3, 7.3, 9.5 Hz, 1H), 2.99-2.91 (m, 1H), 2.80-2.69 (m, 1H), 2.64 (dt, J=3.7, 6.1 Hz, 1H), 2.37 (dt, J=7.6, 9.7 Hz, 1H), 2.26 (m, 1H), 2.13-2.00 (m, 1H), 1.93 (m, 1H), 1.63-1.41 (m, 4H), 1.41-1.24 (m, 2H)

### Example 3

### N-(5-(3-(N-(oxetan-3-yl)sulfamoyl)azetidin-1-yl)pentyl)-5-(thiophen-2-yl)isoxazole-3-carboxamide

### Step 1: benzyl 3-(acetylthio)azetidine-1-carboxylate

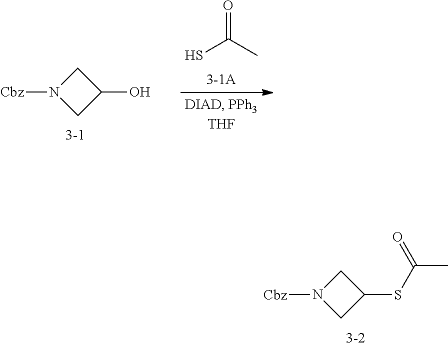

To a solution of PPh₃ (15.82 g, 60.32 mmol, 1.25 eq) in THF (60 mL) at −78° C. was added DIAD (11.9 g, 58.87 mmol, 1.22 eq) in THF (40 mL). After 10 min, thiolacetic acid (4.78 g, 4.48 mL, 62.73 mmol, 1.3 eq) in THF (40 mL) was added followed by, after 10 min, compound 3-1 (10 g, 48.26 mmol, 1.0 eq) in THF (60 mL). The mixture was stirred at −78° C. for 1 hour and then allowed to warm to 23° C. and stirred for 14 hours. TLC (hexane/EtOAc 3:1) showed the reaction was completed. The reaction was quenched with brine (100 mL). The layers were separated. The aqueous phase was extracted with EtOAc (20 mL*3). The combined organic phase was dried over Na₂SO₄, filtered. The filtrate was concentrated under reduced pressure. The residue was purified by column chromatography on silica gel (PE/EA 20:1 to 5:1) to afford compound 3-2 (4.0 g, 31.2% yield) as a light yellow oil. ¹H NMR (400 MHz, CDCl₃) δ ppm 7.37˜7.34 (m, 5H), 5.10 (s, 2H), 4.48-4.44 (m, 2H), 4.24-4.21 (m, 1H), 3.93-3.89 (m, 2H), 2.34 (s, 3H).

### Step 2: benzyl 3-(N-(oxetan-3-yl)sulfamoyl)azetidine-1-carboxylate

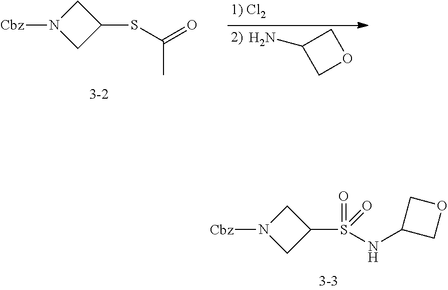 [SMILES: CC(=O)SC1CN(C(=O)OCc2ccccc2)C1 ; NC1COC1 ; O=C(OCc1ccccc1)N1CC(S(=O)(=O)NC2COC2)C1]

To a solution of compound 3-2 (1.5 g, 5.65 mmol, 1.0 eq) in CH₂Cl₂ (20 mL) was added water (5 mL). The mixture was cooled to 0° C. and chlorine gas was bubbled through at 0˜10° C. with stirring for 3 hours. TLC (petroleum ether/EtOAc 3:1) showed the reaction was completed. The layers were separated. The organic phase was washed with brine (20 mL*3). The organic phase was used directly without further purification in the next step. To this crude solution (equivalent to 207 mg, 2.83 mmol, 3 eq) in CH₂Cl₂ (10 mL) was added Et₃N (477 mg, 4.71 mmol, 5 eq) followed by addition of amino oxatane (0.273 g, 0.942 mmol, 1 eq) in CH₂Cl₂ (10 mL) at 0° C. The mixture was stirred at 20-24° C. for 14 hours. LCMS showed the reaction was completed. The volatile was removed under reduced pressure, the residue was purified by prep-HPLC (Column: Xtimate C18 150*25 mm*5 um, gradient: 2050% B (A=0.05% ammonia hydroxide B=CAN), flow rate: 25 mL/min) to afford the title compound (160 mg, 52.03% yield) as a light yellow oil. LC-MS: [M+Na]⁺=349.1.

### Step 3: N-(oxetan-3-yl)azetidine-3-sulfonamide

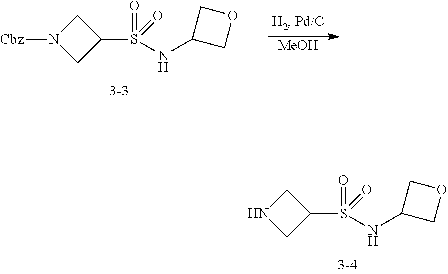 [SMILES: "MeOH" ; O=C(OCc1ccccc1)N1CC(S(=O)(=O)NC2COC2)C1 ; O=S(=O)(NC1COC1)C1CNC1]

To an autoclave was added compound 3-3 (160 mg, 0.490 mmol, 1 eq) and MeOH (15 mL) followed by addition of Pd/C (104 mg, 5% wt) under N₂. The reaction was stirred at 19-23° C. for 18 hours under H2 (15 psi). LCMS showed the starting material was consumed. The suspension was filtered through a pad of celite and the pad was washed with MeOH (10 mL*4). The combined filtrates were concentrated to dryness to give product 3 (92 mg, 97.62% yield) as a colorless oil. LC-MS: [M+Na]⁺=193.1.

### Step 4: N-(5-(3-(N-(oxetan-3-yl)sulfamoyl)azetidin-1-yl)pentyl)-5-(thiophen-2-yl)isoxazole-3-carboxamide

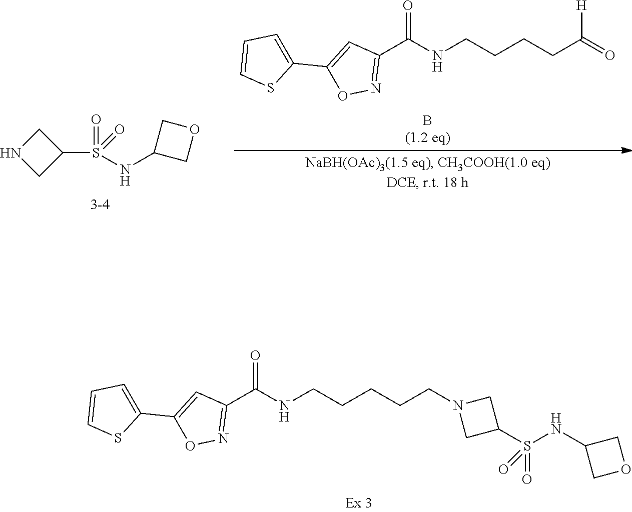 [SMILES: O=CCCCCNC(=O)c1cc(-c2cccs2)on1 ; O=S(=O)(NC1COC1)C1CNC1 ; O=C(NCCCCCN1CC(S(=O)(=O)NC2COC2)C1)c1cc(-c2cccs2)on1]

To a solution of compound 3-4 (92 mg, 478.58 umol, 1.3 eq) in DCE (5 mL) and MeOH (1 mL) was added Intermediate B (100 mg, 0.359 mmol, 1 eq) and HOAc (22 mg, 0.359 mmol, 1.0 eq) followed by addition of NaBH(OAc)₃ (114 mg, 0.538 mmol, 1.5 eq) at 0° C. and the reaction was stirred at 16-21° C. for 18 hours. LCMS showed the reaction was completed. The reaction was quenched with water (1 mL) and concentrated. The residue was dissolved with MeOH (3 mL) and purified by prep-HPLC (Column: Kromasil 150*25 mm*10 um, gradient: 25-55% B (A=(0.05% ammonia hydroxide v/v), B═CH₃CN), Flow Rate (mL/min) 30) to give Example 3 (22.8 mg 13.96% yield) as a white solid. ¹H NMR (400 MHz, CDCl₃) δ ppm 7.56 (dd, J=0.8, 3.6 Hz, 1H), 7.51 (dd, J=0.8, 5.2 Hz, 1H), 7.16 (dd, J=3.6, 4.8 Hz, 1H), 6.95 (brs, 1H), 6.85 (s, 1H), 5.23-5.15 (m, 1H), 4.92-4.88 (m, 2H), 4.75-4.65 (m, 1H), 4.62-4.57 (m, 2H), 3.95-3.85 (m, 1H), 3.60-3.52 (m, 2H), 3.50-3.42 (m, 2H), 3.40-3.35 (m, 2H), 2.49-2.46 (m, 2H), 1.68-1.55 (m, 2H), 1.45-1.35 (m, 4H). LC-MS: [M+H]⁺=455.1.

### Example 4

### N-(5-(3-(N-(2-cyanoethyl)sulfamoyl)azetidin-1-yl)pentyl)-5-(thiophen-2-yl)isoxazole-3-carboxamide

### Step 1: N-(5-(3-sulfamoylazetidin-1-yl)pentyl)-5-(thiophen-2-yl)isoxazole-3-carboxamide

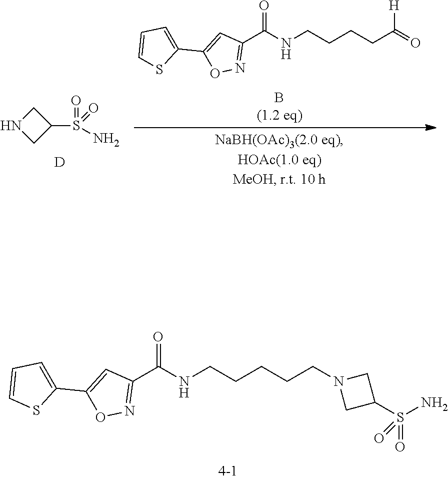

To a mixture of Intermediate B (200 mg, 0.719 mmol, 1.0 eq) in MeOH (4 mL) was added Intermediate D (98 mg, 0.719 mmol, 1.0 eq), HOAc (43 mg, 0.719 mmol, 1.0 eq), NaBH(OAc)₃ (305 mg, 1.44 mmol, 2.0 eq) at 12-46° C. The mixture was stirred at 1216° C. for 14 hours. LCMS showed the reaction was completed. Two parallel reactions with same scale were carried out. The reaction mixture was purified by basic pre-HPLC (Xtimate C18 150*25 mm*5 um, gradient: 26-56% B (A=water (0.05% ammonia hydroxide v/v), B═CH₃CN), flow rate: 25 mL/min) to afford the title compound. (209.2 mg, 73.1% yield as a white solid). ¹H NMR (400 MHz, CD₃OD) δ ppm 7.69-7.66 (m, 2H), 7.21 (dd, J=4.8 Hz, 8.8 Hz, 1H), 6.91 (s, 1H), 4.05-4.03 (m, 1H), 3.65-3.63 (m, 2H), 3.47-3.45 (m, 2H), 3.39-3.37 (m, 2H), 2.56-2.52 (m, 2H), 1.67-1.60 (m, 2H), 1.42-1.39 (m, 4H). LC-MS: [M+H]⁺=399.0.

### Step 2: N-(5-(3-(N-(2-cyanoethyl)sulfamoyl)azetidin-1-yl)pentyl)-5-(thiophen-2-yl)isoxazole-3-carboxamide

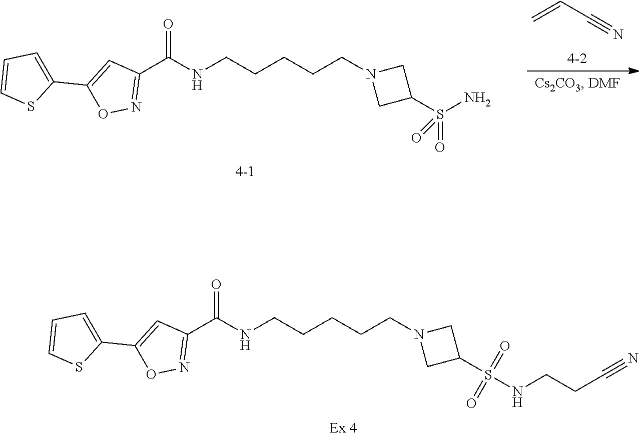

To a suspension of Compound 4-1 (80 mg, 0.201 mmol, 1 eq) in DMF (4.4 mL) and compound 4-2 (10.7 mg, 0.201 mmol, 1 eq) was added Cs₂CO₃ (327 mg, 1.0 mmol, 5 eq) at 8-14° C. The reaction vessel was sealed and heated via microwave at 100° C. for 20 min. LCMS showed the reaction was completed. The reaction was filtered. The filtrate was purified by prep-HPLC (Column Xtimate C18 150*25 mm*5 um, gradient: 26-56% B (A=water (0.05% ammonia hydroxide v/v) B=MeCN), Flow Rate=25 ml/min) to give the title compound (13 mg, 14.34% yield) as an off-white solid. ¹H NMR (400 MHz, CD₃OD) δ ppm 7.71-7.68 (m, 2H), 7.21 (dd, J=4.0, 5.2 Hz, 1H), 6.93 (s, 1H), 4.17-4.12 (m, 1H), 3.68-3.60 (m, 2H), 3.48-3.31 (m, 6H), 2.66 (t, J=6.4 Hz, 2H), 2.57-2.49 (m, 2H), 1.69-1.59 (m, 2H), 1.48-1.37 (m, 4H). LC-MS: [M+H]⁺=452.1.

### Example 5

### 2-(methylamino)-2-oxoethyl (1-(5-(5-(thiophen-2-yl)isoxazole-3-carboxamido)pentypazetidin-3-yl)carbamate

### Step 1: tert-butyl (1-(5-(5-(thiophen-2-yl)isoxazole-3-carboxamido)pentypazetidin-3-yl)carbamate

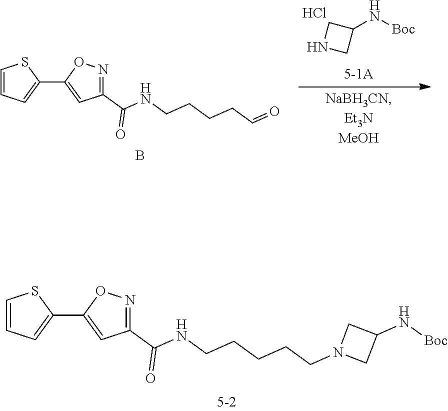

To a mixture of Compound 5-1A (900 mg, 4.31 mmol, 1.2 eq) in MeOH (20 mL) was added Et₃N (473 mg, 4.67 mmol, 1.3 eq) and Intermediate B (1.0 g, 3.59 mmol, 1.0 eq). The mixture was stirred at 26° C. for 1 hour. NaBH₃CN (452 mg, 7.19 mmol, 2.0 eq) was added at 0˜5° C. The mixture was stirred at 25° C. for 14 hours. LCMS indicated the reaction was completed. The mixture diluted with CH₂Cl₂ (40 mL). The organic phase was washed with 10 mL of water. The aqueous layers were extracted with CH₂Cl₂ (20 mL*3). The combined organic phase was dried over Na₂SO₄, filtered. The filtrate was concentrated to give a residue, which was purified by column chromatography on silica gel (PE/EA 10:1 to 1:1) to afford the title compound (0.81 g, 51.9% yield) as a light yellow solid. LC-MS: [M+H]⁺=435.1.

### Step 2: N-(5-(3-aminoazetidin-1-yl)pentyl)-5-(thiophen-2-yl)isoxazole-3-carboxamide

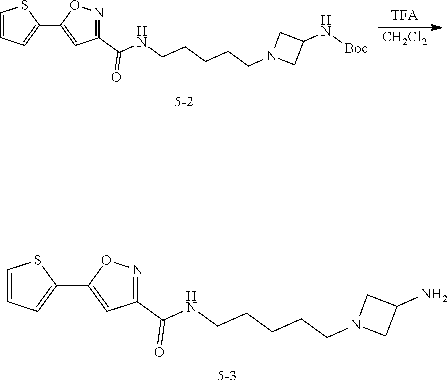

To a solution of Compound 5-2 (0.475 g, 1.09 mmol, 1 eq) in CH₂Cl₂ (12 mL) was added TFA (4 mL). The mixture was stirred at 26-35° C. for 18 hours. LCMS indicated the reaction was completed. The volatile was removed under reduced pressure to afford Compound 5-3 (crude, 100% yield), which was used in the next step without further purification. LC-MS: [M+H]⁺=335.1.

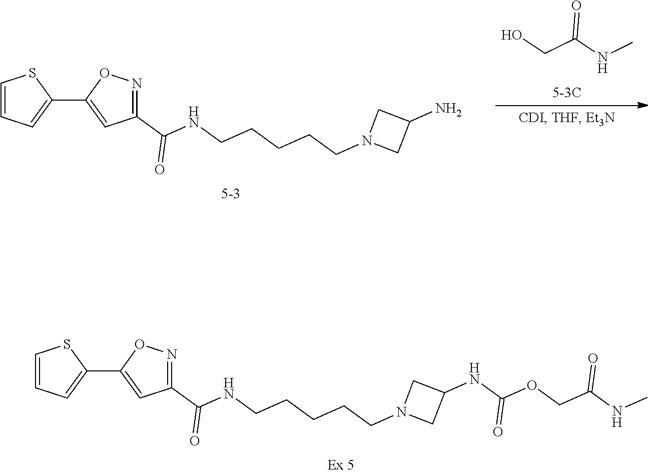

To a solution of Compound 5-3 (100 mg, 0.299 mmol, 1.0 eq) in THF (3 mL) was added Et₃N (151 mg, 1.50 mmol, 5 eq) and CDI (485 mg, 2.99 mmol, 10.0 eq). The mixture was stirred at 26-35° C. for 1 hour. Compound 5-3C (266 mg, 2.99 mmol, 10.0 eq) was added. The mixture was stirred at 60° C. for 15 hours. LCMS showed the reaction was completed. The volatile was removed under reduced pressure. The residue was purified by basic pre-HPLC (Xtimate C18 150*25 mm*Sum, gradient: 16-46% B (A=water (0.05% ammonia hydroxide v/v) to afford the title compound (18.3 mg, 13.39% yield) as a white solid. ¹H NMR (400 MHz, CD₃OD) δ ppm 7.71-7.68 (m, 2H), 7.21 (t, J=4.0 Hz, 1H), 6.93 (s, 1H), 4.48 (s, 2H), 4.26-4.25 (m, 1H), 3.71-3.70 (m, 2H), 3.42-3.40 (m, 2H), 3.10-3.04 (m, 2H), 2.78 (s, 3H), 2.51-2.49 (m, 2H), 1.65-1.63 (m, 2H), 1.44-1.43 (m, 4H). LC-MS: [M+H]⁺=450.2.

### Example 6

### N-(5-(3-sulfamoylpiperidin-1-yl)pentyl)-5-(thiophen-2-yl)isoxazole-3-carboxamide

### Step 1: benzyl 3-((methylsulfonyl)oxy)piperidine-1-carboxylate

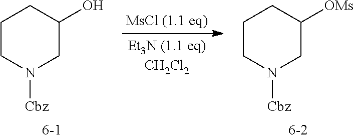 [SMILES: O=C(OCc1ccccc1)N1CCCC(O)C1 ; CS(=O)(=O)OC1CCCN(C(=O)OCc2ccccc2)C1]

To a solution of Compound 6-1 (5.0 g, 21.25 mmol, 1.0 eq) in CH₂Cl₂ (50 mL) was added Et₃N (2.37 g, 23.38 mmol, 1.1 eq) and MsCl (2.68 g, 23.38 mmol, 1.10 eq) at 0° C. After addition, the reaction mixture was warmed to 7-16° C. slowly and stirred at 7-16° C. for 18 hours. LCMS showed the reaction was completed. The organic phase was washed with saturated aqueous NaHCO₃ (30 mL) and brine (30 mL), dried over Na₂SO₄ and filtered. The filtrate was concentrated to give the title compound (5.4 g, crude) as a brown oil. LC-MS: [M+Na]⁺=336.0.

### Step 2: benzyl 3-(acetylthio)piperidine-1-carboxy late

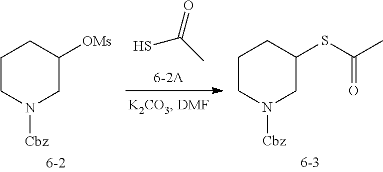

Compound 6-2A (1.97 g, 25.85 mmol, 1.5 eq) was added to a suspension of K₂CO₃ (4.76 g, 34.46 mmol, 2.0 eq) in DMF (50 mL) at 0° C., followed by addition of Compound 2 (5.40 g, 17.23 mmol, 1.0 eq). The resulting mixture was heated at 55° C. for 14 hours. LCMS showed the reaction was completed. The mixture was poured into water (150 mL) and extracted with EtOAc (200 mL). The organic layer was washed with water (100 mL*2) and brine (100 mL). The organic layer was concentrated and purified by column chromatography on silica gel (PE/EA 50:1 to 1:1) to afford the title compound (2.1 g) as a brown oil. LC-MS: [M+H]⁺=294.1.

### Step 3: benzyl 3-(chlorosulfonyl)piperidine-1-carboxylate

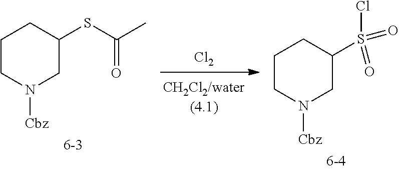 [SMILES: CC(=O)SC1CCCN(C(=O)OCc2ccccc2)C1 ; "Cl2" ; O=C(OCc1ccccc1)N1CCCC(S(=O)(=O)Cl)C1]

To a solution of Compound 6-3 (2.1 g, 7.15 mmol, 1.0 eq) in CH₂Cl₂ (40 mL) was added water (8 mL) at 0° C. and the reaction mixture was bubbled through chlorine gas for 3.5 hours. TLC (petroleum ether/EtOAc 5:1) showed most of Compound 6-3 was consumed. The organic layer was separated and dried over Na₂SO₄, filtered. The filtrate was used for the next step directly as a solution.

### Step 4: benzyl 3-sulfamoylpiperidine-1-carboxylate

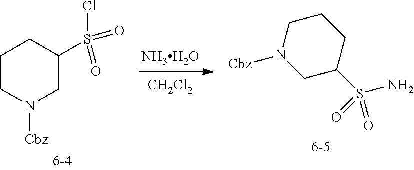

To a solution of compound 6-4 (7.15 mmol, 1.0 eq) in CH₂Cl₂ (40 mL) was added NH₃.H₂O (100 mL) at 0° C. The reaction mixture was stirred for 18 hours at 1317° C. as monitored by LCMS. The mixture was concentrated in vacuum. The residue was dissolved in MeOH (90 mL) and purified by acidic pre-HPLC (Column Boston Green ODS 150*30 5 u, gradient: 20-50% B (A=water (0.1% TFA v/v) B═CH₃CN), Flow Rate 30 ml/min) to afford the title compound (210 mg, 41.3% yield) as a light yellow solid. LC-MS: [M+H]⁺=299.0.

### Step 5: piperidine-3-sulfonamide

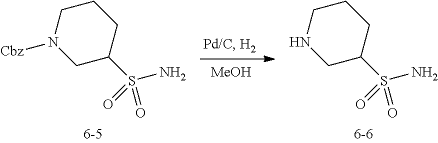 [SMILES: "MeOH" ; NS(=O)(=O)C1CCCN(C(=O)OCc2ccccc2)C1 ; "Pd/C, H2" ; NS(=O)(=O)C1CCCNC1]

To a stirred solution of compound 6-5 (136 mg, 0.456 mmol, 1.0 eq) in MeOH (10 mL) was added Pd/C (100 mg, 10% wt, wet) under N₂. The reaction was stirred at 13-19° C. for 14 hours under hydrogen atmosphere (15 psi). LCMS showed the starting material was consumed. The suspension was filtered through a pad of celite, and the pad was washed with MeOH (10 mL*3). The combined filtrate was concentrated to dryness to give compound 6-6 (78 mg) as a white solid. LCMS: 5-95AB_220 &254 chromatography (MK RP-18e 25-2 mm).

### Step 6: N-(5-(3-sulfamoylpiperidin-1-yl)pentyl)-5-(thiophen-2-yl)isoxazole-3-carboxamide

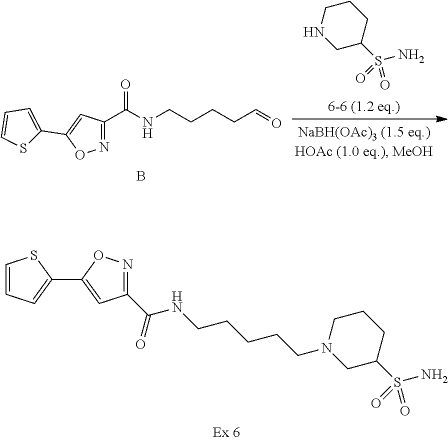

To a solution of compound 6-6 (78 mg, 0.475 mmol, 1 eq) in MeOH (2 mL) was added Intermediate B (132 mg, 0.475 mmol, 1 eq) and HOAc (29 mg, 0.475 mmol, 1.0 eq) followed by addition of NaBH(OAc)₃ (151 mg, 0.712 mmol, 1.5 eq) at 0° C. and the reaction was warmed to 8-14° C., and then it was stirred at 8-14° C. for 18 hours. LCMS showed the reaction was completed. The reaction was quenched with water (1 mL) and concentrated. The residue was diluted with MeOH (2 mL) and purified by prep-HPLC (Column Xtimate C18 150*25 mm*5 um, gradient: 25-55% B (A=water (0.05% ammonia hydroxide v/v) B=CAN), Flow Rate (ml/min) 25) to give the title compound (69 mg, 34.06% yield) as a white solid. ¹H NMR (400 MHz, CD₃OD) δ ppm 7.71-7.68 (m, 2H), 7.23 (dd, J=4.0, 5.2 Hz, 1H), 6.93 (s, 1H), 3.43-3.33 (m, 3H), 3.15-3.05 (m, 1H), 2.98-2.94 (m, 1H), 2.49-2.43 (m, 2H), 2.25-2.17 (m, 1H), 2.10 (t, J=10.8 Hz, 1H), 1.99-1.90 (m, 1H), 1.88-1.80 (m, 1H), 1.70-1.35 (m, 8H). LC-MS: [M+H]⁺=427.2.

### Example 7

### N-(5-(3-sulfamoylpyrrolidin-1-yl)pentyl)-5-(thiophen-2-yl)isoxazole-3-carboxamide

### Step 1: benzyl 3-((methylsulfonyl)oxy)pyrrolidine-1-carboxylate

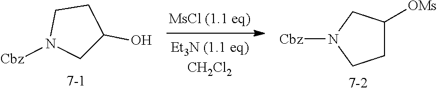

To a solution of compound 7-1 (1.0 g, 4.52 mmol, 1.0 eq) in CH₂Cl₂ (15 mL) was added Et₃N (572 mg, 5.65 mmol, 1.25 eq) and MsCl (622 mg, 5.42 mmol, 1.20 eq) at 0˜5° C. The mixture was stirred at 1113° C. for 14 hours. TLC (PE/EA 3:1) showed the reaction was completed. The organic phase was washed with brine (20 mL*3). The organic phase was dried over Na₂SO₄, filtered. The filtrate was concentrated under reduced pressure to afford the title compound (1.5 g, 100% yield, 90% wt) as a light yellow oil. ¹H NMR (400 MHz, CDCl₃) δ ppm 7.30˜7.30 (m, 5H), 5.07 (s, 2H), 3.78-3.68 (m, 1H), 3.66-3.58 (m, 3H), 3.57-3.56 (m, 1H), 3.04 (s, 3H), 2.32-2.27 (m, 1H), 2.18-2.15 (m, 1H).

### Step 2: benzyl 3-(acetylthio)pyrrolidine-1-carboxylate

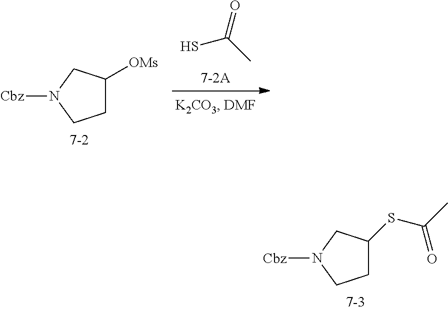

To a solution of compound 7-2 (1.5 g, 4.51 mmol, 1.0 eq) in DM (15 mL) was added K₂CO₃ (935 mg, 6.76 mmol, 1.5 eq) and ethanethioic acid (515 mg, 6.76 mmol, 1.5 eq). The resulting mixture was heated at 70° C. for 14 hours. TLC (hexane/EtOAc 3:1) showed the reaction was completed. The solvent was removed under reduced pressure. The residue was portioned between EtOAc (20 mL) and water (20 mL). The aqueous phase was extracted with EtOAc (20 mL*3). The combined organic phase was dried over Na₂SO₄, filtered. The filtrate was concentrated. The residue was purified by column chromatography on silica gel (PE/EA 30:1 to 3:1) to afford two spot, which have same HNMR, as chocolate-brown oil. (Spot 1 (0.3 g) and spot 2 (0.2 g), overall yield: 39.6%). ¹H NMR (400 MHz, CDCl₃) δ ppm 7.37˜7.32 (m, 5H), 5.15 (s, 2H), 4.01-3.98 (m, 1H), 3.87-3.82 (m, 1H), 3.54-3.50 (m, 2H), 3.34-3.31 (m, 1H), 2.34 (s, 3H), 2.33-2.27 (m, 1H), 1.92-1.89 (m, 1H).

### Step 3: benzyl 3-(chlorosulfonyl)pyrrolidine-1-carboxylate

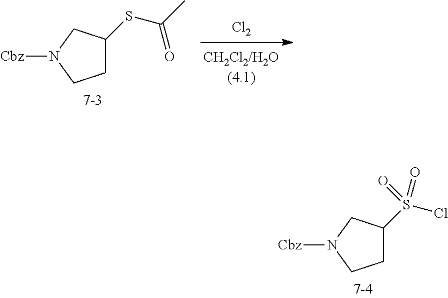

To a solution of compound 7-3 (0.5 g, 1.79 mmol, 1.0 eq) in CH₂Cl₂ (20 mL) was added water (5 mL). The mixture was cooled to 0° C. and chlorine gas was bubbled through with stirring for 3.5 hours. TLC (petroleum ether/EtOAc 3:1) indicated the reaction was completed. The mixture was washed with brine (20 mL*3). The layers were separated to afford a light yellow solution containing compound 4 (1.79 mmol, 100% yield) in CH₂Cl₂ (20 mL), which was used in the next step.

### Step 4: benzyl 3-sulfamoylpyrrolidine-1-carboxylate

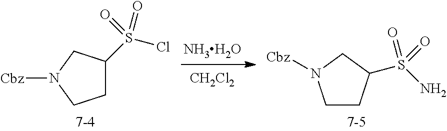

To a solution of NH₃.H₂O (50 mL, 0.359 mol, 28% wt, D=0.9, 200 eq) was added a solution of compound 7-4 (1.79 mmol, 1.0 eq) in CH₂Cl₂ (20 mL) at 0˜5° C. The mixture was stirred at 1622° C. for 48 hours. LCMS showed the reaction was completed. The volatile was removed under reduced pressure. The residue was purified by acidic pre-HPLC (Boston Green ODS 150*30 5 u, gradient: 23-33% B (A=water (0.1% TFA), B═CH₃CN), flow rate: 30 mL/min) to afford the title compound (210 mg, 41.3% yield) as a light yellow solid. ¹H NMR (400 MHz, CD₃OD) δ ppm 7.36˜7.33 (m, 5H), 5.14 (s, 2H), 4.92 (brs, 1H), 4.79 (brs, 1H), 3.82-3.71 (m, 4H), 3.54-3.51 (m, 1H), 3.20-3.10 (m, 1H), 2.35-2.33 (m, 1H). LC-MS: [M+I-]⁺=285.0.

### Step 5: pyrrolidine-3-sulfonamide

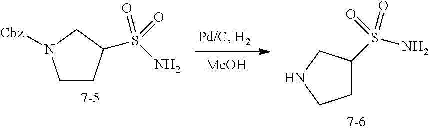 [SMILES: "MeOH" ; NS(=O)(=O)C1CCN(C(=O)OCc2ccccc2)C1 ; "Pd/C, H2" ; NS(=O)(=O)C1CCNC1]

To a solution of compound 7-5 (0.21 g, 0.738 mmol, 1.0 eq) in MeOH (10 mL) was added Pd/C (100 mg, 10% wt, wet). The mixture was stirred under hydrogen atmosphere (15 psi) at 4˜9° C. for 14 hours. LCMS showed the reaction was completed. The mixture was filtered, and the cake was washed with MeOH (10 mL*2). The combined organic phase was concentrated under reduced pressure to afford the title compound (100 mg, 90.2% yield) as a white solid.

### Step 6: N-(5-(3-sulfamoylpyrrolidin-1-yl)pentyl)-5-(thiophen-2-yl)isoxazole-3-carboxamide

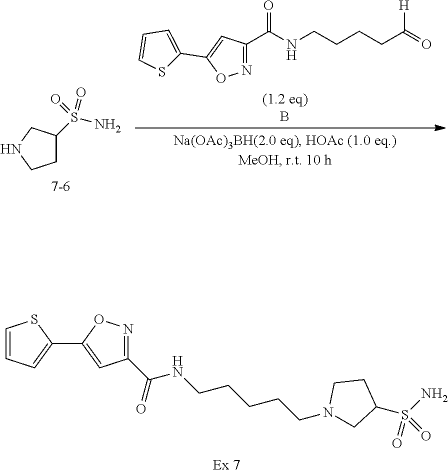 [SMILES: O=CCCCCNC(=O)c1cc(-c2cccs2)on1 ; NS(=O)(=O)C1CCNC1 ; "(1.2 eq)B" ; "Na(OAc)3BH(2.0 eq), HOAc (1.0 eq.)MeOH, r.t. 10 h" ; NS(=O)(=O)C1CCN(CCCCCNC(=O)c2cc(-c3cccs3)on2)C1]

To a solution of Intermediate B (0.1 g, 0.359 mmol, 1.0 eq) in MeOH (2 mL) was added compound 7-6 (54 mg, 0.359 mmol, 1.0 eq), HOAc (22 mg, 0.359 mmol, 1.0 eq), NaBH(OAc)₃ (152.3 mg, 0.718 mmol, 2.0 eq) at 7-13° C. for 14 hours. LCMS showed the reaction was completed. The reaction was quenched by water (0.5 mL). The mixture was purified by basic pre-HPLC (Xtimate C18 150*25 mm*5 um, gradient: 26-56% B (A=water (0.05% ammonia hydroxide v/v), B═CH₃CN), flow rate: 25 mL/min) to afford the title compound (53.6 mg, 36.1% yield) as a light brown solid. ¹H NMR (400 MHz, CD₃OD) δ ppm 7.69-7.66 (m, 2H), 7.21 (dd, J=4.8 Hz, 8.4 Hz, 1H), 6.92 (s, 1H), 4.20-3.96 (m, 2H), 3.85-3.75 (m, 1H), 3.67-3.46 (m, 1H), 3.47-3.35 (m, 2H), 3.30-3.20 (m, 3H), 2.70-2.55 (m, 1H), 2.45-2.35 (m, 1H), 1.85-1.80 (m, 2H), 1.75-1.65 (m, 2H), 1.52-1.39 (m, 2H). LC-MS: [M+H]⁺=413.2.

### Example 8

### N-(5-(3-carbamoylpyrrolidin-1-yl)pentyl)-5-(4-fluorophenyl)isoxazole-3-carboxamide

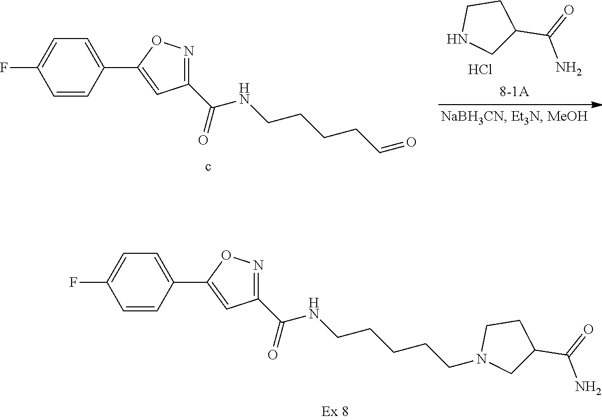

To a solution of compound 8-1A (75 mg, 0.496 mmol, 1.2 eq) in MeOH (2 mL) was added Et₃N (54 mg, 0.537 mmol, 1.3 eq), followed by addition of Intermediate C (120 mg, 0.413 mmol, 1.0 eq) at 5-11° C. The mixture was stirred for 1 hour. NaBH₃CN (52 mg, 0.826 mmol, 2.0 eq) was added and the reaction was stirred at 5-11° C. for 18 hours. LCMS showed the reaction was completed. The reaction was quenched with water (2 mL) and diluted with MeOH (2 mL). The mixture was purified by basic prep-HPLC (Column Kromasil 150*25 mm*10 um, gradient: 20-50% B (A=(0.05% ammonia hydroxide v/v) B═CH₃CN), Flow Rate: 30 ml/min) to afford the title compound (11.4 mg 7.1% yield) as a white solid. ¹H NMR (400 MHz, CD₃OD) δ ppm 7.95-7.92 (m, 2H), 7.30-7.26 (m, 2H), 7.06 (s, 1H), 3.42-3.38 (m, 2H), 3.05-2.90 (m, 2H), 2.85-2.75 (m, 1H), 2.65-2.45 (m, 4H), 2.15-1.95 (m, 2H), 1.70-1.55 (m, 4H), 1.48-1.38 (m, 2H). LC-MS: [M+H]⁺=389.2.

### Example 9

### N-(5-(3-carbamoyl-3-(hydroxymethyl)azetidin-1-yl)pentyl)-5-(thiophen-2-yl)isoxazole-3-carboxamide

### Step 1: diethyl 2,2-bis(hydroxymethyl)malonate

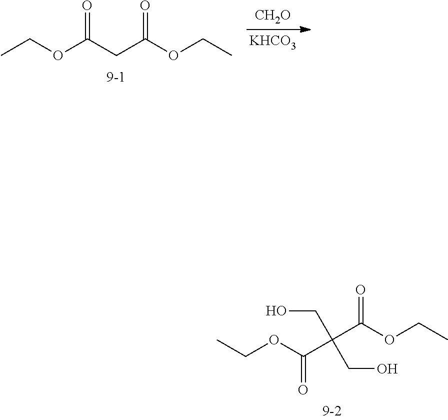

To a mixture of KHCO₃ (500 mg, 4.99 mmol, 0.08 eq) in aq.CH₂O (37%, 16 mL) was added compound 9-1 (10 g, 62.43 mmol, 1.0 eq) at 0° C. The mixture was stirred for 16 hours at 28° C. To the mixture was added water (50 mL) and EtOAc (50 mL). The organic layer was dried over Na₂SO₄, filtered and concentrated under reduced pressure to give the title compound (13 g, crude) as a yellow oil. ¹H NMR (400 MHz, CDCl₃) δ ppm 4.34-4.19 (m, 4H), 4.17-4.06 (m, 4H), 1.36-1.19 (m, 6H).

### Step 2: diethyl 1-benzylazetidine-3,3-dicarboxylate

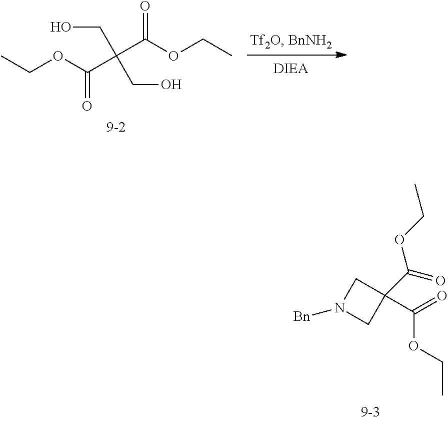

To a mixture of compound 9-2 (8 g, 36.33 mmol, 1.0 eq) in MeCN (160 mL) was added Tf2O (21.52 g, 76.29 mmol, 2.1 eq) at −20° C., followed by DIEA (23.48 g, 181.64 mmol, 5.0 eq). After 0.5 h, BnNH2 was added at −20° C. The mixture was stirred for 2 h at 70° C. EA (200 mL) and brine (100 mL) were added. The organic layer was dried over Na₂SO₄, filtered and concentrated under reduced pressure to a residue. The residue was purified by column chromatography on silica gel (PE:EA=20:1) to give the tile compound (2.7 g). LC-MS: [M+H]⁺=292.3.

### Step 3: ethyl 1-benzyl-3-(hydroxymethyl)azetidine-3-carboxylate

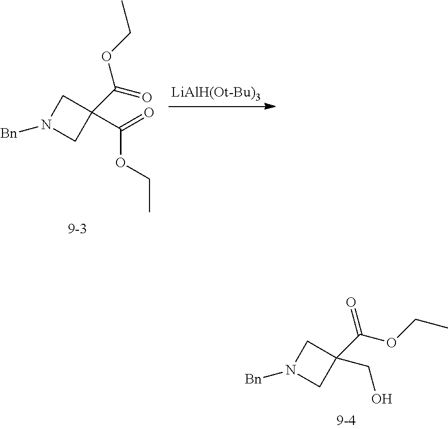

To a solution of compound 9-3 (2.5 g, 8.58 mmol, 1.0 eq) in THF (50 mL) was added LiAlH(Ot-Bu)3 (10.91 g, 42.90 mmol, 5.0 eq) at 0° C. The mixture was stirred for 7 h at 30° C. To sat.NH₄Cl (500 mL) was added the mixture and EA (250 mL). The aqueous layer was extracted with EA (100 mL). The combined EA layers was washed with brine (200 mL), dried over Na₂SO₄, filtered and concentrated under reduced pressure to a residue. The residue was purified by column chromatography on silica gel (PE:EA=10:1 to 1:1) to give the title compound (1.2 g, 49% yield) as a yellow oil. LC-MS: [M+H]⁺=250.3.

### Step 4: 1-benzyl-3-(hydroxymethyl)azetidine-3-carboxamide

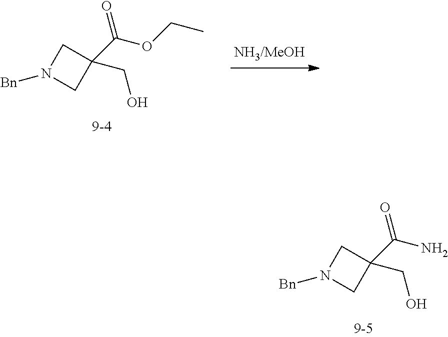 [SMILES: CCOC(=O)C1(CO)CN(Cc2ccccc2)C1 ; NC(=O)C1(CO)CN(Cc2ccccc2)C1]

A mixture of compound 9-4 (500 mg, 2.21 mmol, 1.0 eq) in NH₃/MeOH (15M, 20 mL) in a sealed tube was stirred for 16 hrs at 20° C. and 24 hrs at 35° C. The mixture was concentrated under reduced pressure to give compound 5 (500 mg, crude) as a yellow oil, which was confirmed by LCMS. LCMS: t_{R}=0.686 min MS (ESI) m/z 221.3 [M+H]⁺.

### Step 5: 3-(hydroxymethyl)azetidine-3-carboxamide

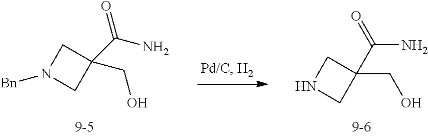 [SMILES: NC(=O)C1(CO)CN(Cc2ccccc2)C1 ; NC(=O)C1(CO)CNC1]

To a mixture of compound 9-5 (500 mg, 2.27 mmol, 1.0 eq) in MeOH (10 mL) was added Pd/C (200 mg, wet, 10%) at 30° C. The mixture was stirred for 4 hours at 30° C. under H2 (15 psi). The mixture was filtered and the filtrate was concentrated under reduced pressure to give the title compound (300 mg. crude) as a yellow oil.

### Step 6: N-(5-(3-carbamoyl-3-(hydroxymethyl)azetidin-1-yl)pentyl)-5-(thiophen-2-yl)isoxazole-3-carboxamide

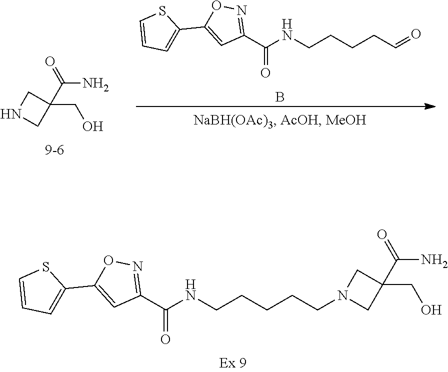

To a mixture of compound 9-6 (150 mg, crude), Intermediate B (213.86 mg, 768.37 umol, 1.0 eq) and AcOH (46.14 mg, 768.37 umol, 1.0 eq) in MeOH (4 mL) were added NaBH(OAc)3 (325.7 mg, 1.54 mmol, 2.0 eq) at 30° C. The mixture was stirred for 3 h at 30° C. To the mixture was added sat.NaHCO₃ (0.5 mL). The mixture was filtered and the filtrate was purified by Prep-HPLC (base) to give the title compound (108.93 mg, 100% purity, 36.1% yield) as a white solid. ¹H NMR (400 MHz, CD₃OD) δ ppm 7.69 (m, 2H), 7.23 (dd, J=3.7, 4.9 Hz, 1H), 6.92 (s, 1H), 3.88 (s, 2H), 3.44-3.36 (m, 4H), 3.34 (m, 2H), 2.51 (t, J=7.1 Hz, 2H), 1.65 (quin, J=7.0 Hz, 2H), 1.50-1.30 (m, 4H). LC-MS: [M+H]⁺=393.3.

### Example 10

### N-(5-(3-carbamoyl-3-(hydroxymethyl)azetidin-1-yl)pentyl)-5-(4-fluorophenyl)isoxazole-3-carboxamide

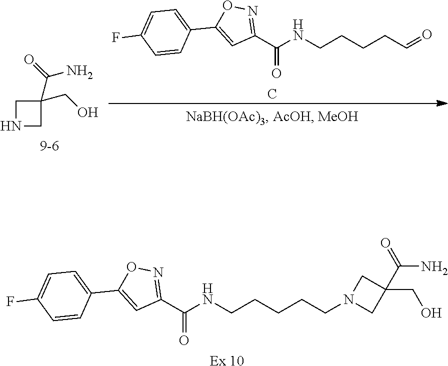

To a mixture of compound 9-6 (150 mg, crude), Intermediate C (223.05 mg, 768.37 umol, 1.0 eq) and AcOH (46.14 mg, 768.37 umol, 1.0 eq) in MeOH (4 mL) were added NaBH(OAc)3 (325.7 mg, 1.54 mmol, 2.0 eq) at 30° C. The mixture was stirred for 3 h at 30° C. To the mixture was added sat.NaHCO₃ (0.5 mL). The mixture was filtered and the filtrate was purified by Prep-HPLC (base) to give the title compound (85.96 mg, 99% purity, 27.5% yield) as a white solid. ¹H NMR (400 MHz, CD₃OD) δ ppm 8.00-7.88 (m, 2H), 7.36-7.23 (m, 2H), 7.07 (s, 1H), 3.88 (s, 2H), 3.45-3.36 (m, 4H), 3.36-3.33 (m, 2H), 2.51 (t, J=7.1 Hz, 2H), 1.66 (m, 2H), 1.51-1.31 (m, 4H). LC-MS: [M+H]⁺=405.4.

### Example 11

### N-(5-(3-carbamoyl-3-methylazetidin-1-yl)pentyl)-5-(thiophen-2-yl)isoxazole-3-carboxamide

### Step 1: 1-benzyl 3-ethyl 3-methylazetidine-1,3-dicarboxylate

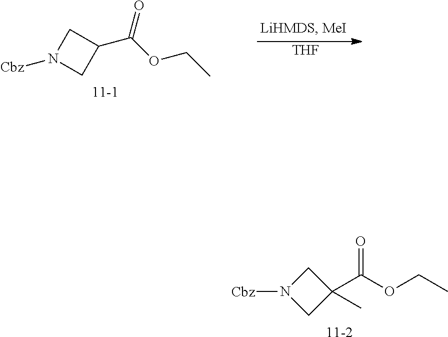

To a solution of compound 11-1 (1 g, 4.01 mmol, 1.0 eq) and MeI (1.14 g, 8.02 mmol, 2.0 eq) in THF (20 mL) was added LiHMDS (1M, 8 mL) at −70° C. The mixture was allowed to warm to 25° C. and stirred for 16 h. To sat.NH₄Cl (200 mL) was added the mixture and EA (150 mL). The organic layer was washed with brine (100 mL), dried over Na₂SO₄, filtered. The filtrate was concentrated under reduced pressure to give compound 11-2 (550 mg, crude) as a yellow oil. LCMS: t_{R}=0.882 min MS (ESI) m/z 264.0 [M+H]⁺.

### Step 2: benzyl 3-carbamoyl-3-methylazetidine-1-carboxy late

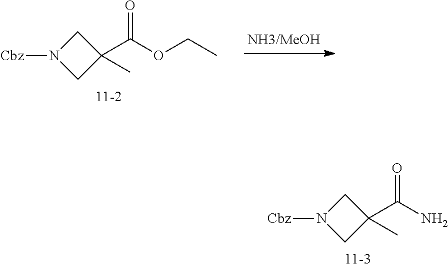

To compound 11-2 (550 mg, 2.09 mmol) was added NH₃/MeOH (7M, 30 mL) at 25° C. The mixture was stirred for 16 hat 25° C. and 6 h at 35° C. The mixture was concentrated under reduced pressure and to give compound 11-3 (500 mg, crude) as a yellow oil. LCMS: t_{R}=0.761 min [M+Na]⁺270.9.

### Step 3: 3-methylazetidine-3-carboxamide

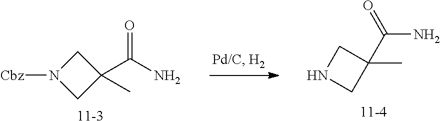

To a solution of compound 11-3 (500 mg, 20.01 mmol) in MeOH (10 mL) was added Pd/C (wet, 10%, 1 g) at 25° C. The mixture was stirred at 25° C. under H2 at 15 psi for 16 hours. The mixture was filtered. The filtrate was concentrated under reduced pressure to give the title compound (250 mg, crude) as a yellow oil.

### Step 4: N-(5-(3-carbamoyl-3-methylazetidin-1-yl)pentyl)-5-(thiophen-2-yl)isoxazole-3-carboxamide

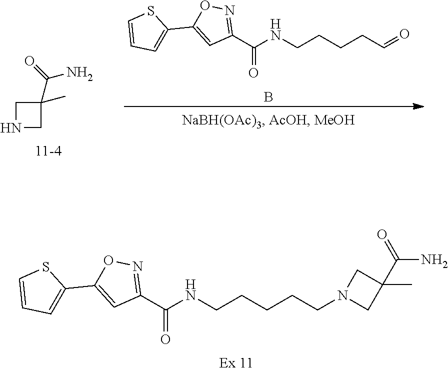

To a mixture of compound 11-4 (120 mg, crude) and Intermediate B (146.3 mg, 525.64 umol, 1.0 eq) in MeOH (3 mL) were added HOAc (31.57 mg, 525.64 umol, 1.0 eq) and NaHB(OAc)₃ (222.81 g, 1.05 mmol, 2.0 eq) at 25° C. The mixture was stirred for 3 h at 25° C. To the reaction was added water (0.5 mL). The mixture was filtered and filtrate was purified by basic pre-HPLC (Phenomenex Gemini 150*25 mm*10 um, gradient: 24-54% B (A=water (0.05% ammonia hydroxide v/v), B═CH₃CN), flow rate: 25 mL/min) to give the title compound (43.99 mg, 100%, 22% yield) as a white solid. ¹H NMR (400 MHz, CD₃OD) δ ppm 7.69 (m, 2H), 7.23 (dd, J=3.8, 5.0 Hz, 1H), 6.92 (s, 1H), 3.45 (d, J=8.3 Hz, 2H), 3.40 (t, J=7.0 Hz, 2H), 3.18 (d, J=8.3 Hz, 2H), 2.49 (br t, J=7.2 Hz, 2H), 1.65 (m, 2H), 1.53 (s, 3H), 1.48-1.33 (m, 4H). LC-MS: [M+H]⁺=377.4.

### Example 12

### N-(5-(3-carbamoyl-3-methylazetidin-1-yl)pentyl)-5-(4-fluorophenyl)isoxazole-3-carboxamide

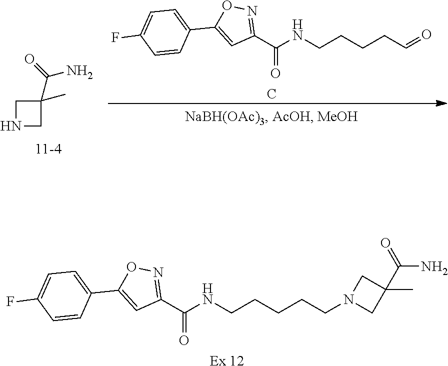

To a mixture of compound 11-4 (120 mg, crude) and Intermediate C (152.59 mg, 525.64 umol, 1.0 eq) in MeOH (3 mL) were added HOAc (31.57 mg, 525.64 umol, 1.0 eq) and NaHB(OAc)₃ (222.81 g, 1.05 mmol, 2.0 eq) at 25° C. The mixture was stirred for 3 h at 25° C. To the reaction was added water (0.5 mL). The mixture was filtered and filtrate was purified by basic pre-HPLC (Phenomenex Gemini 150*25 mm*10 um, gradient: 26-56% B (A=water (0.05% ammonia hydroxide v/v), B═CH₃CN), flow rate: 25 mL/min) to afford the title compound (65.5 mg, 100%, 32% yield) as a white solid. ¹H NMR (400 MHz, CD₃OD) δ ppm 8.00-7.89 (m, 2H), 7.35-7.24 (m, 2H), 7.07 (s, 1H), 3.49-3.37 (m, 4H), 3.18 (d, J=8.0 Hz, 2H), 2.49 (t, J=7.2 Hz, 2H), 1.65 (m, 2H), 1.53 (s, 3H), 1.49-1.33 (m, 4H). LC-MS: [M+H]⁺=389.4.

### Example 13

### N-(5-(3-carbamoyl-4-hydroxypyrrolidin-1-yl)pentyl)-5-(thiophen-2-yl)isoxazole-3-carboxamide

### Step 1: tert-butyl 3-cyano-4-hydroxypyrrolidine-1-carboxylate

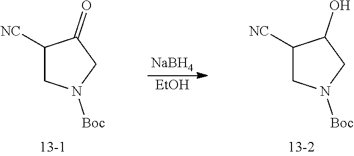 [SMILES: "EtOH" ; CC(C)(C)OC(=O)N1CC(=O)C(C#N)C1 ; "NaBH4" ; CC(C)(C)OC(=O)N1CC(O)C(C#N)C1]

The compound 13-1 in 150 mL of EtOH was cooled to 0° C. To this solution was added portionwise NaBH₄ (1.08 g, 28.54 mmol, 2.0 eq). The mixture was stirred for 30 min at 0° C. The mixture was concentrated under reduced pressure. The residue was diluted with EA (100 mL). To the mixture was added water (50 mL). The EA layer was washed with brine (50 mL), dried over Na₂SO₄ and concentrated under reduced pressure to give the title compound (3.0 crude). 41 NMR (400 MHz, CHLOROFORM-d) δ=4.54 (m, 1H), 3.87-3.63 (m, 2H), 3.46-3.21 (m, 1H), 3.08-2.90 (m, 1H), 2.67 (s, 1H), 1.46-1.31 (s, 9H). LC-MS: [M−55]⁺=157.1.

### Step 2: tert-butyl 3-carbamoyl-4-hydroxypyrrolidine-1-carboxylate

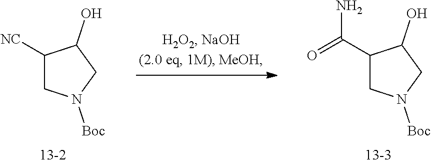 [SMILES: CC(C)(C)OC(=O)N1CC(O)C(C#N)C1 ; CC(C)(C)OC(=O)N1CC(O)C(C(N)=O)C1]

To a solution of compound 13-2 in a mixture of aq NaOH (1N, 20 mL) and MeOH (40 mL) was added H₂O₂ at 10° C. The mixture was stirred at 10° C. for 16 hours. Then sat.NH₄Cl (50 mL) was added to the mixture. The result mixture was extracted with EA (500 mL×4). The organic layer was washed with brine (500 mL), dried over Na₂SO₄ and concentrated under reduced pressure to give the title compound (1 g, crude) as a white solid. LC-MS: [M+Na]⁺=253.3.

### Step 3: 4-hydroxypyrrolidine-3-carboxamide

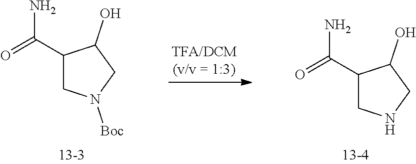 [SMILES: CC(C)(C)OC(=O)N1CC(O)C(C(N)=O)C1 ; NC(=O)C1CNCC1O]

To a mixture of compound 13-3 in DCM (15 mL) was added TFA (3 mL) at 10° C. The mixture was stirred for 16 hours at 10-15° C. The mixture was concentrated under reduced pressure to give the title compound (380 mg, crude) as a yellow oil. LC-MS: [M+H]⁺=131.1.

### Step 4: N-(5-(3-carbamoyl-4-hydroxypyrrolidin-1-yl)pentyl)-5-(thiophen-2-yl)isoxazole-3-carboxamide

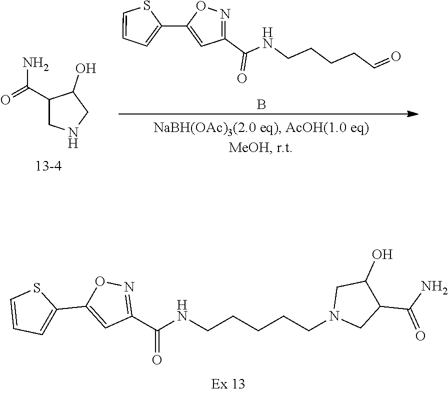

To a mixture of compound 13-4 (350 mg, 2.69 mmol, 1.0 eq), Intermediate B (748.51 mg, 2.69 mmol, 1.0 eq) and HOAc (161.5 mg, 2.69 mmol, 1.0 eq) in MeOH (15 mL) was added NaBH(OAc)₃ (1.14 g, 5.38 mmol, 2.0 eq) at 10° C. The mixture was stirred for 6 hours at 10-15° C. To the mixture was added sat.NaHCO₃ (5 mL). The mixture was concentrated under reduced pressure. The residue was purified by Prep-HPCL (base) (column: Phenomenex Gemini C18 250*50 mm*10 um, gradient: 2045% B (A=water/0.05% ammonia, B═CH₃CN, flow rate: 100 mL/min) to give the title compound (230 mg, 92% purity, 21.7% yield) as a white solid. 41 NMR (400 MHz, METHANOL-d₄) δ=7.69 (m, 2H), 7.22 (dd, J=3.8, 5.0 Hz, 1H), 6.92 (s, 1H), 4.53-4.42 (m, 1H), 3.41 (m, 2H), 3.15-3.05 (m, 1H), 3.05-2.80 (m, 2H), 2.76-2.70 (m, 1H), 2.63-2.39 (m, 3H), 1.73-1.53 (m, 4H), 1.51-1.36 (m, 2H). LC-MS: [M+H]⁺=393.4.

### Example 14

### N-(5-(4-sulfamoylpiperidin-1-yl)pentyl)-5-(thiophen-2-yl)isoxazole-3-carboxamide

### Step 1: benzyl 4-sulfamoylpiperidine-1-carboxylate

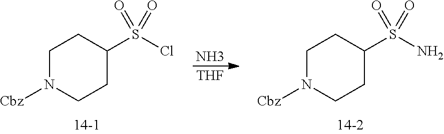

To a mixture of compound 14-1 (0.5 g, 1.57 mmol, 1.0 eq) in THF (10 mL) was bubbled NH₃ at 0° C. for 10 min. The mixture was concentrated. The residue was dissolved into EA (50 mL). The EA layer was washed with water (50 mL) and brine (50 mL). The organic layer was dried over Na₂SO₄, filtered and concentrated to afford the title compound as a white solid. LC-MS: [M+Na]⁺=321.1.

### Step 2: piperidine-4-sulfonamide

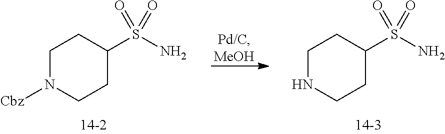 [SMILES: NS(=O)(=O)C1CCN(C(=O)OCc2ccccc2)CC1 ; "Pd/C,MeOH" ; NS(=O)(=O)C1CCNCC1]

A mixture of compound 14-2 (480 mg, 1.61 mmol, 1.0 eq) and Pd/C (100 mg, 10%, wet) in MeOH (10 mL) was stirred under H2 at 50 psi for 6 h at 30° C. LCMS showed desired mass was detected. The mixture was filtered and concentrated to give compound 14-3 (180 mg, crude) as a white solid, which was used for next step directly.

### Step 3: N-(5-(4-sulfamoylpiperidin-1-yl)pentyl)-5-(thiophen-2-yl)isoxazole-3-carboxamide

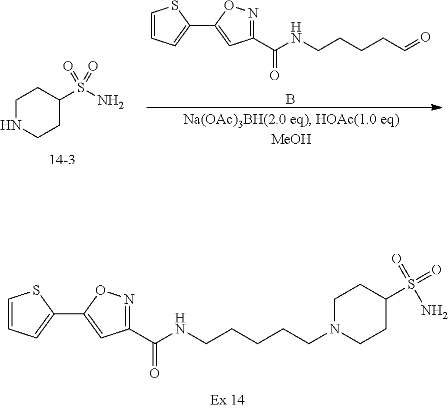

To a mixture of compound 14-3 (160 mg, 574.86 nmol, 1.0 eq) and Intermediate B (94.41 mg, 574.86 nmol, 1.0 eq) in MeOH (4 mL) were added AcOH (34.52, 574.86 nmol, 1.0 eq) and NaBH(OAc)₃ (243.67, 1.15 mmol, 2.0 eq) at 10-15° C. The mixture was stirred for 16 h at 8-15° C. To the reaction was added sat.NaHCO₃ (1 mL) and MeOH (50 mL). The mixture was filtered and filtrate was concentrated under reduced pressure to give a crude product. The crude product was purified by basic prep-HPLC (Phenomenex Gemini 150*25 mm*10 um, gradient: 33-63% B (A=water (0.05% ammonia hydroxide v/v), B═CH₃CN), flow rate: 25 mL/min) to afford the title compound (97.3 mg, 38.6% yield) as a white solid. ¹H NMR (400 MHz, DMSO-d₆) δ=8.81 (t, J=5.9 Hz, 1H), 7.88 (dd, 5.0 Hz, 1H), 7.80 (dd, J=1.1, 3.6 Hz, 1H), 7.28 (dd, J=3.8, 5.0 Hz, 1H), 7.18 (s, 1H), 6.70 (s, 2H), 3.25 (q, J=6.9 Hz, 2H), 2.95 (d, J=11.0 Hz, 2H), 2.75 (m, 1H), 2.26 (t, J=7.3 Hz, 2H), 1.95 (d, J=10.5 Hz, 2H), 1.86 (t, J=11.9 Hz, 2H), 1.66-1.49 (m, 4H), 1.45 (m, 2H), 1.36-1.22 (m, 2H). LC-MS: [M+H]⁺=427.3.

### Example 15

### 5-(4-fluorophenyl)-N-(5-(4-sulfamoylpiperidin-1-yl)pentyl)isoxazole-3-carboxamide

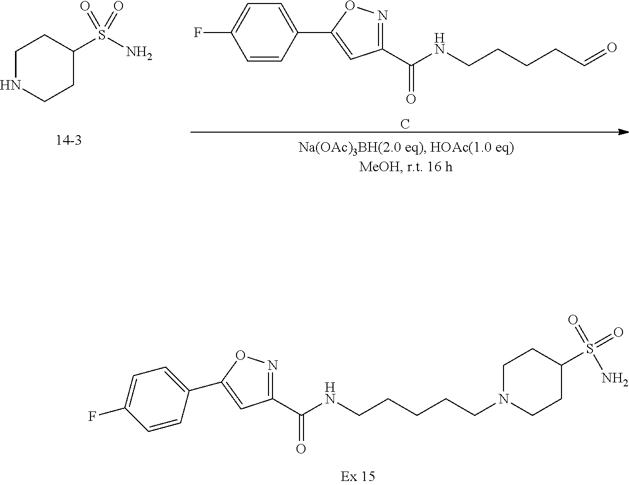

To a mixture of Intermediate C (160 mg, 551.17 nmol, 1.0 eq) and compound 14-3 (90.52 mg, 551.17 nmol, 1.0 eq) in MeOH (4 mL) were added AcOH (33.10 mg, 551.17 nmol, 1.0 eq) and NaBH(OAc)₃ (233.63 mg, 1.10 mmol, 2.0 eq) at 10-15° C. The mixture was stirred for 16 h at 8-15° C. To the reaction was added sat.NaHCO₃ (1 mL) and MeOH (20 mL). The mixture was filtered and filtrate was concentrated under reduced pressure to give a crude product. The crude product was purified by basic prep-HPLC (Phenomenex Gemini 150*25 mm*10 um, gradient: 35-65% B (A=water (0.05% ammonia hydroxide v/v), B═CH₃CN), flow rate: 25 mL/min) to afford the title compound (117.66 mg, 48.7% yield) as a white solid. ¹H NMR (400 MHz, DMSO-d₆) δ ppm 8.82 (t, J=5.8 Hz, 1H), 8.06-7.94 (m, 2H), 7.48-7.38 (m, 2H), 7.35 (s, 1H), 6.70 (br s, 2H), 3.30-3.21 (m, 2H), 2.95 (d, J=11.3 Hz, 2H), 2.82-2.70 (m, 1H), 2.26 (t, J=7.2 Hz, 2H), 1.95 (d, J=12.3 Hz, 2H), 1.86 (t, J=11.9 Hz, 2H), 1.68-1.50 (m, 4H), 1.49-1.39 (m, 2H), 1.36-1.23 (m, 2H). LC-MS: [M+H]⁺=439.3.

### Example 16

### N-(5-((3R,4R)-4-cyano-3-hydroxypiperidin-1-yl)pentyl)-5-(thiophen-2-yl)isoxazole-3-carboxamide

### Step 1: 3-benzyl-7-oxa-3-azabicyclopioiheptane

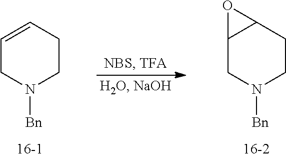 [SMILES: C1=CCN(Cc2ccccc2)CC1 ; c1ccc(CN2CCC3OC3C2)cc1]

compound 16-1 (6.60 g, 38.9 mmol, 1.0 eq) was added dropwised to the mixture of triflouroacetic acid (4.34 g, 38.9 mmol, 1.0 eq) and water (70 mL) and stirred at 26° C. The resulting suspension was stirred for 15 min, NB S (8.81 g, 49.52 mmol, 1.3 eq) was added portionwise to the mixture over 10 min, during the time the temperature was increased to 30-35° C. After been stirred for 16 h at 26° C., a 20% aqueous NaOH (70 mL) was added dropwise to solution, and then the mixture was stirred for 2 h. The reaction mixture was extracted with EtOAc (200 mL*3) dried over Na₂SO₄, the organic layer was concentrated. The residue was purified by silica gel column chromatography (PE/EA=10:1) to afford the title compound. ¹H NMR (400 MHz, CDCl₃) δ ppm 7.22-7.17 (m, 5H), 3.37 (s, 2H), 3.15-3.11 (m, 2H), 2.92-2.91 (m, 1H), 2.61-2.58 (d, J=13.3 Hz, 1H), 2.28-2.22 (m, 1H), 2.15-2.09 (m, 1H), 1.95-1.90 (m, 2H).

### Step 2: 1-benzyl-3-hydroxypiperidine-4-carbonitrile

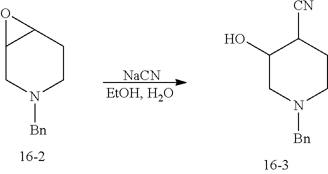

A solution of compound 16-2 (2.0 g, 10.57 mmol, 1.0 eq) and NaCN (1.04 g, 21.14 mmol, 2.0 eq) in EtOH/H2O=5/1 (24 mL) was stirred at 30° C. for 16 h. TLC (PE:EA=2:1) showed that starting materials (Rf=0.6) was still remained and 2 spots was formed (Rf=0.4 and Rf=0.2). And the resulting suspension was stirred for 16 h at 50° C. TLC (PE:EA=2:1) showed that less starting materials was remained, then stop the reaction. The mixture was poured into water (10 mL) and exacted with EA (60 mL), the organic layer was washed with brine (40 mL), dried over Na₂SO₄.

The filtrate was purified by flash column chromatography on silica gel (PE/EA=10:1) to give the the title compound as a colorless oil (550 mg in yield 25%). ¹H NMR (400 MHz, DMSO-d₆) δ ppm 7.3-7.21 (m, 5H), 5.52-5.51, (d, J=5.99, 1H), 3.56-3.51 (m, 2H), 2.88-2.85 (dd, J=10.94, 1H), 2.70-2.67 (m, 2H), 2.47-2.45 (m, 1H), 2.00-1.97 (m, 1H), 1.89-1.88 (m, 1H), 1.74-1.66 (m, 2H).

### Step 3: (3R,4R)-3-hydroxypiperidine-4-carbonitrile

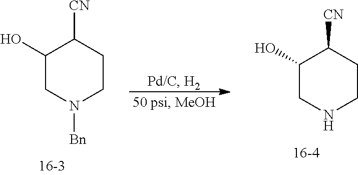 [SMILES: N#CC1CCN(Cc2ccccc2)CC1O ; "Pd/C, H2" ; N#C[C@H]1CCNC[C@@H]1O]

To solution of compound 16-3 (100.0 mg, 0.46 mmol, 1.0 eq) in MeOH (5 mL) was added Pd/C (10 mg). The mixture was purged with H2 gas (50 psi), and stirred at 25° C. for 16 hours. TLC (PE/EA=3:1) showed that starting materials (Rf=0.5) was disappeared and a new spot (Rf=0.1) was formed, The organic layer was filtered and dried in vacuo to give a crude product (20 mg) in 34% yield, which was confirmed by the nmr. ¹H NMR (400 MHz, CDCl₃) δ ppm 3.88-3.84 (m, 1H), 3.26-3.20 (m, 1H), 3.00-2.97 (m, 1H), 2.72-2.69 (m, 2H), 2.15-2.10 (m, 2H), 1.80-1.76 (m, 1H).

### Step 4: N-(5-((3R,4R)-4-cyano-3-hydroxypiperidin-1-yl)pentyl)-5-(thiophen-2-yl)isoxazole-3-carboxamide

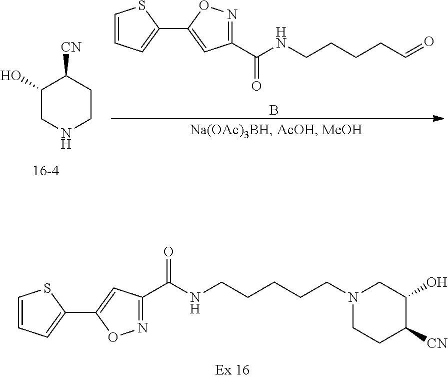

To a 10 ml schlenk tube compound 16-4 (40 mg, 0.3 mmol, 1.0 eq) and Intermediate B (88. mg, 0.3 mmol, 1.0 eq), HOAc (19.4 mg, 0.3 mmol, 1.0 eq) were dissolved in 3 ml MeOH, and NaHB(OAc)₃ (201 mg, 0.9 mmol, 3.0 eq) was added to the mixture under 25° C. stirred for 30 min. TLC (PE/EA=1:1) showed that starting materials (Rf=0.5) was disappeared and a new spot was formed (Rf=0.1). LCMS showed desired mass was detected. The residue was purified by Prep-HPLC (base condition) to afford the title compound as white powder. ¹H NMR (400 MHz, CD₃OD) δ ppm 7.70-7.66 (m, 2H), 7.23-7.21 (dd, J=5.01, 1H), 6.91 (s, 1H), 3.75-3.69 (m, 1H), 3.42-3.39 (t, J=7.09, 2H), 3.04 (m, 1H), 2.86 (m, 1H), 2.50-2.39 (m, 3H), 2.14-2.08 (m, 1H), 2.03-1.95 (m, 1H), 1.91-1.76 (m, 2H), 1.70-1.63 (m, 2H), 1.61-1.54 (m, 2H), 1.45-1.379 (m, 2H). LC-MS: [M+H]⁺=389.2.

### Example 17

### N-(5-(4-(3-amino-3-oxopropyl)piperazin-1-yl)pentyl)-5-(thiophen-2-yl)isoxazole-3-carboxamide

### Step 1: tert-butyl 4-(3-amino-3-oxopropyl)piperazine-1-carboxylate

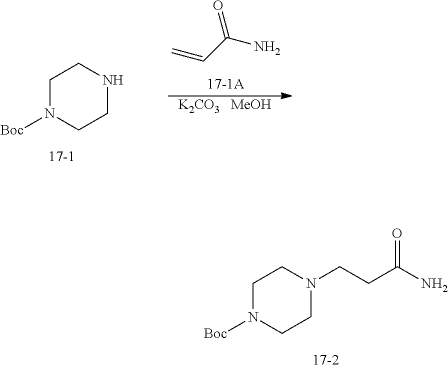

To a mixture of compound 17-1 (200 mg, 1.07 mmol, 1.0 eq) in MeOH (2 mL) was added compound 17-1A (99.22 mg, 1.40 mmol, 1.3 eq) and K₂CO₃ (222.61 mg, 1.61 mmol, 1.5 eq), the reaction mixture was stirred at 25° C. for 5 h. TLC (DCM:MeOH 10:1) showed A (Rf=0.5) was consumed completely, and a new spot (Rf=0.55) was detected. The mixture was concentrated in vacuum. Water (10 ml) and EA (10 ml) was added to the mixture, the organic layer dried over Na₂SO₄ and concentrated in vacuum to afford the title compound (150 mg, crude). It was used in the next step directly. ¹H NMR (400 MHz, CDCl₃) δ ppm 7.81 (s, 1H), 5.59 (s, 1H), 3.50-3.43 (t, J=4.8 4H), 2.71-2.62 (t, J=5.6, 2H), 2.51-2.38 (m, 6H), 1.47 (s, 9H).

### Step 2: 3-(piperazin-1-yl)propanamide

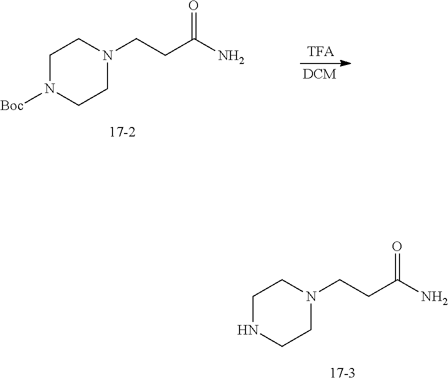 [SMILES: CC(C)(C)OC(=O)N1CCN(CCC(N)=O)CC1 ; NC(=O)CCN1CCNCC1]

To a solution of compound 17-2 (150 mg, 582.91 umol, 1.0 eq) in DCM (1 mL) was added TFA (0.2 ml). The mixture was stirred at 25° C. for 1 h. TLC (MeOH:DCM=1:10) showed A (RF=0.55) was consumed completely and a new spot (RF=0) was directed. The mixture was concentrated in vacuum. Compound 17-3 (120 mg, crude) was obtained as a colorless oil, which would confirmed in the next step.

### Step 3: N-(5-(4-(3-amino-3-oxopropyl)piperazin-1-yl)pentyl)-5-(thiophen-2-yl)isoxazole-3-carboxamide

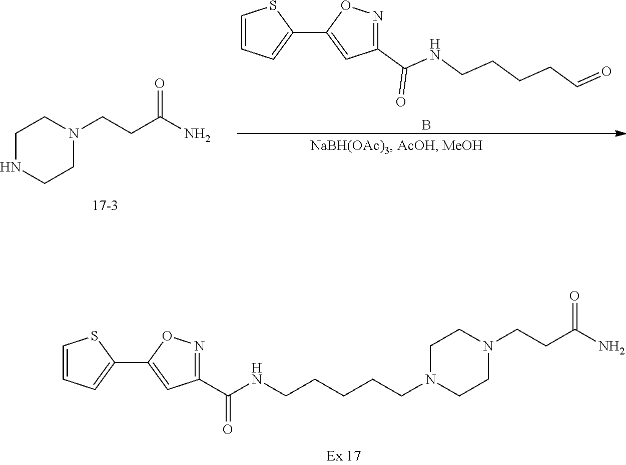

To a mixture of compound 17-3 (120 mg, 763.29 umol, 1.0 eq) in MeOH (1 mL) was added Intermediate B (233.69 mg, 839.62 umol, 1.1 eq), NaBH(CH₃COO)₃ (323.55 mg, 1.53 mmol, 2.0 eq) and CH₃COOH (45.84 mg, 763.29 umol, 1.0 eq). The mixture was stirred at 20° C. for 1 hour. LCMS C-05708-28-P1A1 showed desired mass was detected. The reaction mixture was concentrated in vacuum. MeOH (2 ml) and NaHCO₃ (0.1 g) was added to the mixture, and it was filtered, the crude was purified by Prep-HPLC (NH₃H₂O) to afford the title compound (58 mg, 100% purity, 18.11% yield) as a white solid after lyophilized. ¹H NMR (400 MHz, CDCl₃) δ ppm 8.20 (s, 1H), 7.57 (dd, J=1.0, 3.6 Hz, 1H), 7.52 (dd, J=1.0, 5.0 Hz, 1H), 7.17 (dd, 5.0 Hz, 1H), 6.97-6.81 (m, 1H), 6.84 (s, 1H), 5.31 (s, 1H), 3.48 (m, 2H), 2.74-2.32 (m, 14H), 1.72-1.66 (m, 2H), 1.60-1.52 (m, 2H), 1.49-1.38 (m, 2H). LC-MS: [M+H]⁺=420.4.

### Example 18

### N-(5-(4-(2-amino-2-oxoethyl)piperazin-1-yl)pentyl)-5-(thiophen-2-yl)isoxazole-3-carboxamide

### Step 1: benzyl 4-(2-amino-2-oxoethyl)piperazine-1-carboxylate

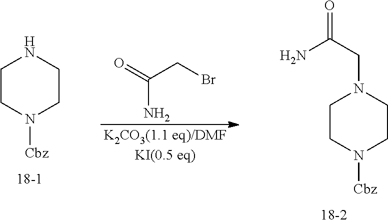 [SMILES: O=C(OCc1ccccc1)N1CCNCC1 ; NC(=O)CBr ; NC(=O)CN1CCN(C(=O)OCc2ccccc2)CC1]

A mixture of compound 18-1 (300 mg, 1.36 mmol, 1.0 eq), 2-bromoacetamide (206.69 mg, 1.50 mmol, 1.1 eq), K₂CO₃ (282.35 mg, 2.04 mmol, 1.5 eq) and KI (113.05 mg, 680.99 μmmol, 0.5 eq) in DMF (5 mL) was stirred for 2 hours at 50° C. The mixture was poured into water (50 mL) and extracted with EA (50 mL×2). The combined organic phase was washed with brine (50 mL), dried over Na2SO4 and filtered. The filtrate was concentrated under reduced pressure to title compound (250 mg) as a white solid, which was used for next step directly.

### Step 2: 2-(piperazin-1-yl)acetamide

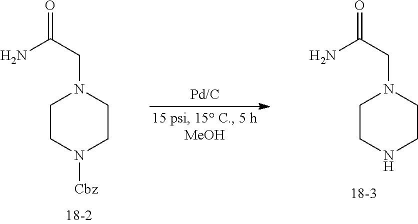 [SMILES: NC(=O)CN1CCN(C(=O)OCc2ccccc2)CC1 ; NC(=O)CN1CCNCC1]

A mixture of compound 18-2 (0.25 g, 901.49 μmol) and Pd/C (50 mg, wet, 10%) in MeOH (5 mL) was stirred for 4 hours under H2 at 15 psi at 10-15° C. The mixture was filtered and the filtrate was concentrated to give a crude product (140 mg). LC-MS: [M+H]⁺=144.2.

### Step 3: N-(5-(4-(2-amino-2-oxoethyl)piperazin-1-yl)pentyl)-5-(thiophen-2-yl)isoxazole-3-carboxamide

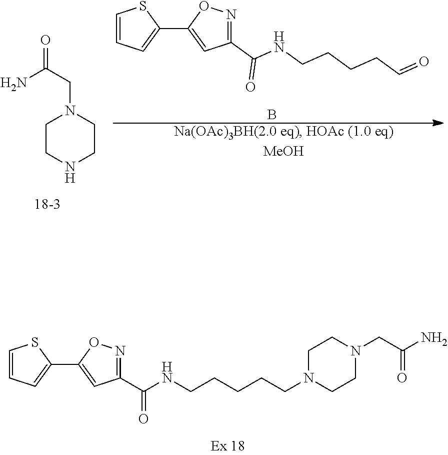

To a mixture of Intermediate B (225 mg, 808.40 μmol, 1.0 eq), Compound 18-3 (115.75 mg, 808.40 μmol, 1.0 eq) and HOAc (48.55 mg, 808.40 μmol, 1.0 eq) in MeOH (5 mL) was added NaBH(OAc)₃ (342.67 mg, 1.62 mmol, 2.0 eq) at 10° C. The mixture was stirred for 16 hours at 10-15° C. To the mixture was added Na₂CO₃ (5 mL) and MeOH (50 mL). The result mixture was filtered and concentrated to give a residue. The residue was purified by Prep-HPLC (base) (column: Phenomenex Gemini C18 250*50 mm*10 um, gradient: 3262% B (A=water/0.05% ammonia, B═CH3CN, flow rate: 25 mL/min) to give the title compound (169.21 mg, 49% yield) as a white solid. ¹H NMR (400 MHz, DMSO-d₆) δ ppm 8.80 (t, J=5.8 Hz, 1H), 7.88 (dd, 5.0 Hz, 1H), 7.80 (dd, J=1.1, 3.6 Hz, 1H), 7.28 (dd, J=3.5, 5.0 Hz, 1H), 7.18 (s, 1H), 7.10 (s, 2H), 3.25 (q, J=6.8 Hz, 2H), 2.82 (s, 2H), 2.49-2.29 (m, 8H), 2.25 (t, J=7.3 Hz, 2H), 1.53 (m, 2H), 1.44 (m, 2H), 1.35-1.23 (m, 2H). LC-MS: [M+H]⁺=406.3.

### Example 19

### N-(5-(4-(2-sulfamoylethyl)piperazin-1-yl)pentyl)-5-(thiophen-2-yl)isoxazole-3-carboxamide

### Step 1: ethenesulfonamide

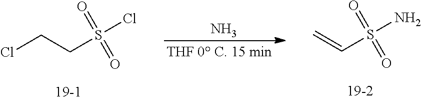

Ammonia was bubbled through a solution of Compound 19-1 (2.5 g, 15.34 mmol) in THF (40 mL) at 0° C. for 15 min, then water (20 mL) was added to the reaction mixture. The resulting solution was extracted with EA (50 mL*4). The combined organic layer was washed with brine (40 mL), dried over Na₂SO₄, filtered and concentrated to afford the title compound (800 mg, crude) as yellow oil. The residue was used for next step without purification. ¹H NMR (400 MHz, DMSO-d₆) δ ppm 7.05 (s, 2H), 6.85-6.73 (m, 1H), 5.98 (d, J=16.4 Hz, 1H), 5.83 (d, J=10.0 Hz, 1H).

### Step 2: tert-butyl 4-(2-sulfamoylethyl)piperazine-1-carboxylate

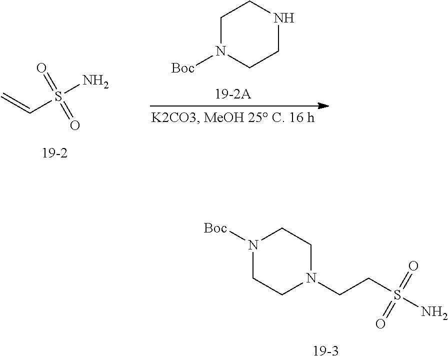

To a solution of Compound 19-2 (200 mg, crude) and Compound 19-2A (268 mg, 1.44 mmol) in MeOH (4 mL) was added K₂CO₃ (298 mg 2.16 mmol). The reaction mixture was stirred at 25° C. for 16 hours. TLC (PE:EA=5:1) showed most of the staring material (Rf=0.1) was consumed and one new spot (Rf=0.5) was observed, The reaction mixture was diluted with water (10 mL), extracted with EA (10 mL*5). The organic phase was washed with brine (30 mL), dried over Na₂SO₄, filtered and concentrated. The residue was purified by column chromatography (SiO₂, PE:EA=50:1 to 1:1) to afford the title compound (310 mg, 73% yield) as yellow solid. 41 NMR (400 MHz, CDCl₃) δ ppm 5.26 (br s, 2H), 3.47-3.40 (m, 4H), 3.31-3.16 (m, 2H), 2.99-2.83 (m, 2H), 2.55-2.42 (m, 4H), 1.45 (s, 9H).

### Step 3: 2-(piperazin-1-yl)ethane-1-sulfonamide

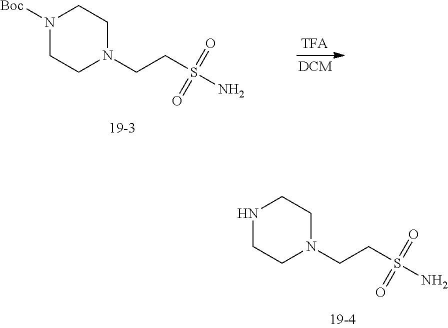 [SMILES: CC(C)(C)OC(=O)N1CCN(CCS(N)(=O)=O)CC1 ; NS(=O)(=O)CCN1CCNCC1]

To a mixture of Compound 19-3 (300 mg, 1.02 mmol) in DCM (4 mL) was added TFA (1 ml). The reaction mixture was stirred at 20° C. for 4 hrs. TLC (EA) showed Compound 19-3 (R_{f}=0.7) was consumed completely, and one new spot (Rf=0.05) was observed. The reaction mixture was concentrated to give the title compound (400 mg, crude) as yellow oil. The residue was used for next step directly without purification.

### Step 4: N-(5-(4-(2-sulfamoylethyl)piperazin-1-yl)pentyl)-5-(thiophen-2-yl)isoxazole-3-carboxamide

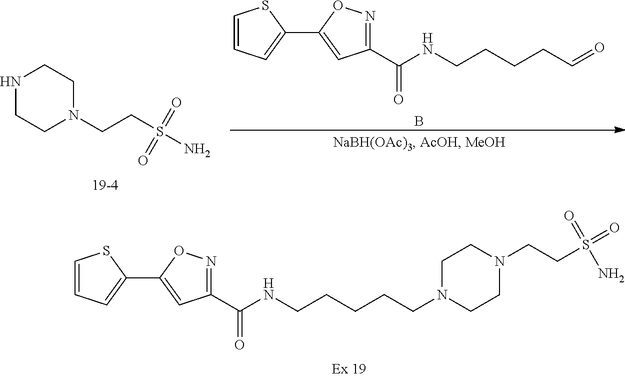

To a mixture of Compound 18-4 (400 mg, crude) and Intermediate 19 (258 mg, 926 μmop in MeOH (9 mL), wad added NaBH(OAc)₃ (393 mg, 1.85 mmol) and AcOH (56 mg, 926 μmop. The reaction mixture was stirred at 20° C. for 5 hours. LCMS (C-05668-99-P1A1) showed Compound 19-4 was almost completed the desired MW was observed. The reaction mixture was quenched by water (20 ml), extracted with EA (20 ml*3), the organic layer were washed with brine (40 ml), dried over Na₂SO₄. The residue were purified by prep-HPLC (base) to give the title compound (69.98 mg, 16% yield) as white solid. ¹H NMR (400 MHz, CDCl₃) δ ppm 7.57 (d, J=3.20 Hz, 1H), 7.52 (d, J=5.20 Hz, 1H), 7.18 (t, J=4.00 Hz, 1H), 6.88-6.85 (m, 1H), 6.84 (s, 1H), 5.34 (s, 2H), 3.54-3.43 (m, 2H), 3.25 (t, J=7.50 Hz, 2H), 2.94 (m, J=6.40 Hz, 2H), 2.76-2.39 (m, 8H), 2.36 (t, J=7.20 Hz, 2H), 1.70-1.67 (m, 2H), 1.58-1.51 (m, 2H), 1.47-1.37 (m, 2H). LC-MS: [M+H]⁺=456.3.

### Example 20

### N-(5-(4-cathamoylpiperidin-1-yl)pentyl)-5-(thiophen-2-yl)isoxazole-3-carboxamide

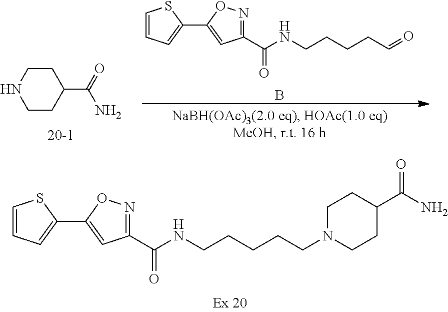

To a solution of Compound 20-1 (300 mg, 2.34 mmol, 1.0 eq) and Intermediate B (651 mg, 2.34 mmol, 1.0 eq) in MeOH (8 mL) was added HOAc (140 mg, 2.34 mmol, 1.0 eq), NaBH(OAc)₃ (992 mg, 4.68 mmol, 2.0 eq) at 0° C. The reaction mixture was stirred at 15° C. for 16 hrs. LCMS showed most of Intermediate B was consumed and desired MW was observed as a main peak. The reaction mixture was quenched with saturated aqueous NaHCO₃ (20 mL) and extracted with EA (10 mL*3). The combined organic layer was dried over Na₂SO₄, filtered and concentrated. The residue was purified by Pre-HPLC (NH₃.H₂O) to afford the title compound (161 mg, 18% yield, 99% purity) as white solid. ¹H NMR (400 MHz, DMSO-d₆) δ ppm 8.79 (m, 1H), 7.88 (d, J=4.8 Hz, 1H), 7.80 (d, J=2.8 Hz, 1H), 7.29-7.27 (m, 1H), 7.17 (s, 2H), 6.69 (s, 1H), 3.27-3.22 (m, 2H), 2.84 (d, J=11.6 Hz, 2H), 2.22 (t, J=6.8 Hz, 2H), 2.02-1.92 (m, 1H), 1.81 (t, J=10.4 Hz, 2H), 1.64 (m, 2H), 1.58-1.40 (m, 6H), 1.35-1.16 (m, 2H). LC-MS: [M+H]⁺=391.3.

### Example 21

### N-(5-(3-carbamoyl-3-hydroxyazetidin-1-yl)pentyl)-5-(thiophen-2-yl)isoxazole-3-carboxamide

### Step 1

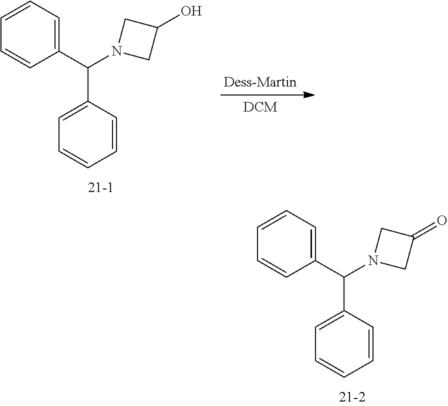 [SMILES: OC1CN(C(c2ccccc2)c2ccccc2)C1 ; O=C1CN(C(c2ccccc2)c2ccccc2)C1]

To a solution of compound 21-1 (5 g, 20.89 mmol, 1.0 eq) and Na₂CO₃ (8.78 g, 104.47 mmol, 5.0 eq) in DCM (100 mL) was added Dess-Martin (17.8 g, 41.8 mmol, 2.0 eq) at 0° C. The mixture was allowed to warm to 30 and stirred for 3 h. The organic layer was separated, dried over Na₂SO₄ and concentrated under reduced pressure to give a residue, which was purified by MPLC to afford the title compound 21-2 (1.8 g, crude) as a yellow oil. 41 NMR (400 MHz, CDCl₃) δ ppm 7.56-7.43 (m, 5H), 7.36-7.28 (m, 5H), 4.64 (s, 1H), 4.06 (s, 4H).

### Step 2: 1-benzhydryl-3-((trimethylsilyl)oxy)azetidine-3-carbonitrile

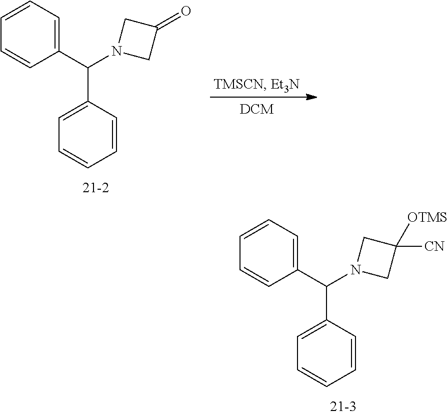

To compound 21-2 (200 mg, 842.83 ummol, 1.0 eq) and Et3N (127.93 mg, 1.26 mmol, 1.5 eq) in DCM (1 mL) was added TMSCN (209.03 mg, 2.11 mmol, 2.0 eq) at 30° C. The mixture was stirred for 3 h at 30° C. The mixture was concentrated under reduced pressure and to afford the title compound 21-3 (350 mg, crude) as a yellow solid. LC-MS: [M+H]⁺=337.3.

### Step 3: 1-benzhydryl-3-hydroxyazetidine-3-carboxamide

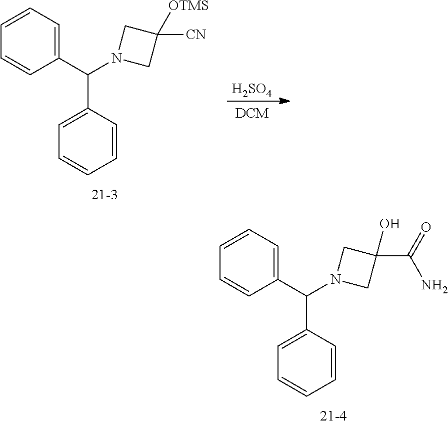

To a solution of compound 21-3 (350 mg, crude) in DCM (2 mL) was added H2504 (0.2 mL) at 0° C. The mixture was allowed to warm to 30° C. and stirred for 2 h. To the mixture was added NH₃.H₂O to PH=11. The mixture was concentrated under reduced pressure to give a residue, which was purified by Prep-HPLC (base) to give the title compound (58 mg. 98% purity) as white solid. LC-MS: [M+H]⁺=283.3.

### Step 4: 3-hydroxy azetidine-3-carboxamide

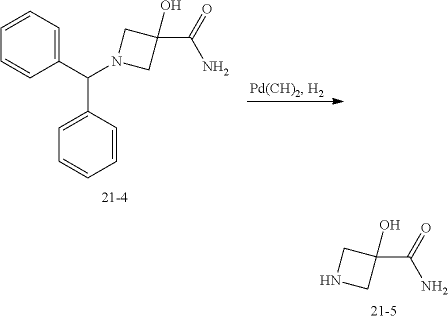 [SMILES: NC(=O)C1(O)CN(C(c2ccccc2)c2ccccc2)C1 ; NC(=O)C1(O)CNC1]

To a solution of compound 21-4 (58 mg, 205.43 umol) in MeOH (1 mL) was added Pd(OH)2 (wet, 10%, 50 mg) at 30° C. The mixture was stirred at 30° C. under H2 at 45 psi for 5 hours. The mixture was filtered. The filtrate was concentrated under reduced pressure to give compound 21-5 (20 mg, crude) as a white solid.

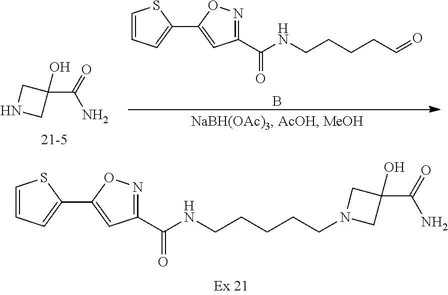

To a mixture of compound 21-5 (20 mg, crude) and Intermediate B (47.94 mg, 172.24 umol, 1.0 eq) in MeOH (1 mL) were added HOAc (10.34 mg, 172.24 umol, 1.0 eq) and NaHB(OAc)₃ (73.01 mg, 344.48 umol, 2.0 eq) at 30° C. The mixture was stirred for 16 h at 30° C. To the reaction was added sat.NaHCO₃ (1 mL). The mixture was concentrated and the residue was purified by basic pre-HPLC (base) to afford the title compound (18.55 mg, 98.9% purity) as a white solid. ¹H NMR (400 MHz, CD₃OD) δ ppm 7.71-7.67 (m, 2H), 7.23 (dd, J=3.7, 4.9 Hz, 1H), 6.92 (s, 1H), 3.63 (d, J=9.3 Hz, 2H), 3.40 (t, J=7.1 Hz, 2H), 3.27 (d, J=9.0 Hz, 2H), 2.57 (t, J=7.2 Hz, 2H), 1.66 (m, 2H), 1.52-1.32 (m, 4H). LC-MS: [M+H]⁺=379.3.

### Example 22

### 5-(5-fluorothiophen-2-yl)-N-(5-(3-(methylcarbamoyl)azetidin-1-yl)pentyl)isoxazole-3-carboxamide

### Step 1: 5-fluoro-N-methoxy-N-methylthiophene-2-carboxamide

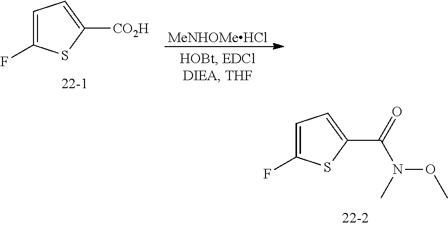

To a solution of compound 22-1 (2.0 g, 13.69 mmol, 1.0 eq.) in THF (150 mL) was added MeNHOMe HCl salt (2.67 g, 27.37 mmol, 2.0 eq), HOBt (1.85 g, 13.69 mmol, 1.0 eq), DIEA (8.84 g, 68.43 mmol, 5.0 eq) under nitrogen atmosphere at 0° C., followed by addition of EDCI (5.25 g, 27.37 mmol, 2.0 eq). The mixture was allowed to warm to 25° C. and stirred at 25° C. for 14 hours. TLC (PE/EA 3:1) showed that the reaction was completed. The reaction was quenched with 50 mL of aq. saturated NaHCO₃. The layers were separated. The aqueous phase was extracted with EtOAc (30 mL*3). The combined organic phase was dried over anhydrous sodium sulfate, filtered. The filtrate was concentrated under reduced pressure. The residue was purified by column chromatography on silica gel (petroleum ether:EtOAc=20:1 to 1:1) to afford the title compound (2.5 g, 96.5% yield) as a light yellow oil. ¹H NMR (400 MHz, CDCl₃) δ ppm 7.68 (t, J=4.0 Hz, 1H), 6.55 (dd, J=1.6 Hz, J=4.0 Hz, 1H), 3.79 (s, 3H), 3.36 (s, 3H).

### Step 2: 1-(5-fluorothiophen-2-yl)ethan-1-one

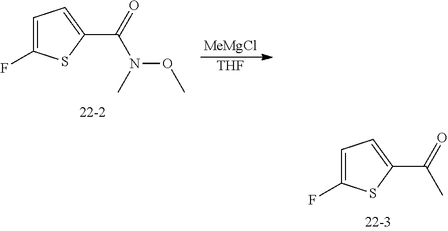

To a stirred solution of compound 22-2 (2.5 g, 13.2 mmol, 1.0 eq.) in THF (30 mL) was added MeMgCl (6.6 mL, 19.8 mmol, 3 M solution in THF, 1.5 eq) at 0° C. under N₂ atmosphere over a period of 20 minutes, while maintaining the internal temperature below 10° C. After addition, the mixture was stirred at 25° C. for 2 hours. LCMS showed the reaction was completed. The reaction mixture was poured into aq. saturated NH₄Cl (30 mL). The layers were separated. The aqueous was extracted with EtOAc (20 mL*3). The combined organic phase was dried over anhydrous sodium sulfate, filtered. The filtrate was concentrated under reduced pressure to give a residue, which was purified by column chromatography on silica gel (petroleum ether:EtOAc=20:1 to 5:1) to afford the title compound (1.6 g, 84.2% yield) as a light yellow oil. ¹H NMR (400 MHz, CDCl₃) δ ppm 7.39 (t, J=4.0 Hz, 1H), 6.55 (dd, J=0.8 Hz, 4.0 Hz, 1H), 2.49 (s, 3H). LC-MS: [M+H]⁺=144.8.

### Step 3: ethyl 4-(5-fluorothiophen-2-yl)-2,4-dioxobutanoate

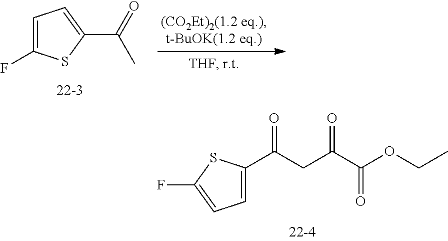

To a solution of compound 22-3 (1.6 g, 11.1 mmol, 1.0 eq.) in THF (30 mL) was added t-BuOK (1.49 g, 13.32 mmol, 1.2 eq.) and (CO₂Et)₂ (1.95 g, 13.32 mmol, 1.2 eq.) at 25° C. The mixture was stirred at 25° C. for 3 hours. TLC (PE/EA 5:1) showed the reaction was completed. The reaction was quenched with 1N HCl to adjust pH to 1-2. The layers were separated. The aqueous phase was extracted with EtOAc (20 mL*3). The combined organic phase was dried over Na₂SO₄, filtered. The filtrate was concentrated under reduced pressure. The residue was purified by column chromatography on silica gel (PE/EA 20:1 to 1:1) to afford the title compound (2.0 g, 73.8% yield) as a light yellow oil. 41 NMR (400 MHz, CDCl₃) δ ppm 7.57 (t, J=4.4 Hz, 1H), 6.85 (s, 1H), 6.62 (d, J=3.2 Hz, 1H), 4.40 (q, J=7.2 Hz, 2H), 1.42 (t, J=7.2 Hz, 3H).

### Step 4: ethyl 5-(5-fluorothiophen-2-yl)isoxazole-3-carboxylate

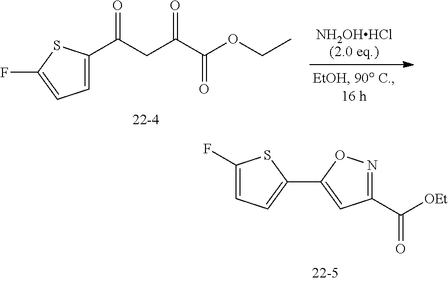 [SMILES: CCOC(=O)C(=O)CC(=O)c1ccc(F)s1 ; CCOC(=O)c1cc(-c2ccc(F)s2)on1]

To a solution of compound 22-4 (2.0 g, 8.19 mmol, 1.0 eq.) in EtOH (20 mL) was added NH₂OH.HCl (683 mg, 9.83 mmol, 1.2 eq.). The mixture was stirred under reflux for 14 hours. LCMS indicated the reaction was completed. The solvent was removed under reduced pressure. The residue was partitioned between aq. saturated NaHCO₃ (20 mL) and EtOAc (20 mL). The layers were separated. The aqueous layers were extracted with EtOAc (20 mL*3). The combined organic phase was dried over anhydrous sodium sulfate, filtered. The filtrate was concentrated. The residue was purified by column chromatography on silica gel (PE/EA=20:1 to 3:1) to give compound 22-5 (1.6 g, 80.8% yield) as a light yellow solid. ¹H NMR (400 MHz, CDCl₃) δ ppm 7.22 (t, J=4.4 Hz, 1H), 6.70 (s, 1H), 6.58 (t, J=2.0 Hz, 1H), 4.47 (q, J=7.2 Hz, 2H), 1.43 (t, J=7.2 Hz, 3H). LC-MS: [M+H]⁺=241.9.

### Step 5: 5-(5-fluorothiophen-2-yl)isoxazole-3-carboxylic acid

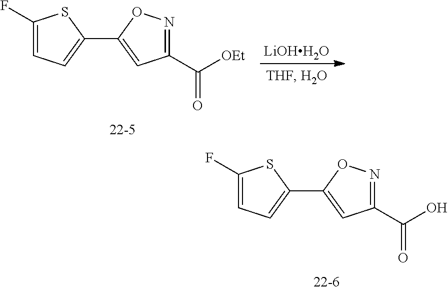

To a solution of compound 22-5 (1.6 g, 6.63 mmol, 1.0 eq.) in THF (10 mL) was added a solution of LiOH.H₂O (557 mg, 13.26 mmol, 2.0 eq.) in water (5 mL). The mixture was stirred at 25° C. for 3 hours. TLC (PE/EA 5:1) showed the reaction was completed. The solvent was removed. The aqueous phase was extracted with EtOAc (20 mL*3). The combined organic phase was dried over anhydrous sodium sulfate, filtered. The filtrate was concentrated under reduced pressure to afford the title compound (1.2 g, 66.7% yield) as yellow solid. 41 NMR (400 MHz, CDCl₃) δ ppm 7.24 (t, J=4.0 Hz, 1H), 6.76 (s, 1H), 6.60 (dd, J=1.2 Hz, 4.0 Hz, 1H).

### Step 6: 5-(5-fluorothiophen-2-yl)-N-(5-hydroxypentyl)isoxazole-3-carboxamide

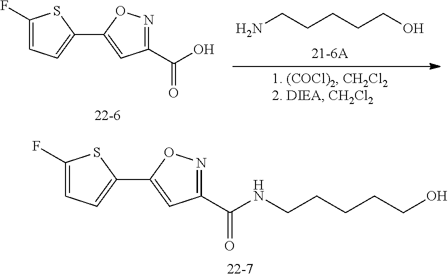 [SMILES: O=C(O)c1cc(-c2ccc(F)s2)on1 ; NCCCCCO ; O=C(NCCCCCO)c1cc(-c2ccc(F)s2)on1]

To a solution of compound 22-6 (0.8 g, 3.75 mmol, 1 eq) in CH₂Cl₂ (10 mL) was added oxalyl chloride (715 mg, 5.63 mmol, 1.5 eq) and 2 drops of DMF at 25° C. The mixture was stirred at 25° C. for 2 hours. The volatile was removed under reduced pressure. The residue was dissolved in CH₂Cl₂ (10 mL), which was transferred into a solution of compound 22-6A (774 mg, 7.51 mmol, 1.5 eq) and Et₃N (1.9 g, 18.76 mmol, 5.0 eq) in CH₂Cl₂ (20 mL) at 0˜5° C. dropwise. The mixture was stirred at 25° C. for 14 hours. LCMS showed the reaction was completed. The reaction was quenched with aq. saturated NaHCO₃ (10 mL). The layers were separated. The aqueous phase was extracted with CH₂Cl₂ (10 mL*3). The combined organic phase was dried over Na₂SO₄, filtered. The filtrate was concentrated under reduced pressure to give the title compound (1 g, 89.2% yield) as a light yellow solid. 41 NMR (400 MHz, CD₃OD) δ ppm 7.60 (t, J=3.6 Hz, 1H), 6.88 (s, 1H), 6.73 (dd, J=2.0 Hz, 4.0 Hz, 1H), 3.56 (t, J=6.4 Hz, 2H), 3.39 (t, J=7.2 Hz, 2H), 1.67-1.57 (m, 4H), 1.46-1.44 (m, 2H). LC-MS: [M+H]⁺=299.0.

### Step 7: N-(5-bromopentyl)-5-(5-fluorothiophen-2-yl)isoxazole-3-carboxamide

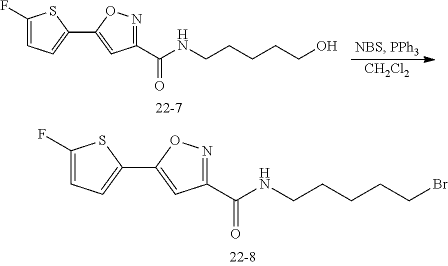

To a solution of compound 22-7 (1 g, 3.35 mmol, 1 eq) in CH₂Cl₂ (20 mL) was added PPh₃ (1.06 g, 4.02 mmol, 2.0 eq) and NBS (0.72 g, 4.02 mmol, 2.0 eq) at 0˜5° C. The mixture was allowed to warm to 25° C. and stirred for 14 hours. LCMS showed the reaction was completed. The reaction was quenched with aq. saturated NaHCO₃ (20 mL). The layers were separated. The aqueous phase was extracted with CH₂Cl₂ (20 mL*3). The combined organic phase was dried over Na₂SO₄, filtered. The filtrate was concentrated under reduced pressure. The residue was purified by column chromatography on silica gel (PE/EA 30:1 to 5:1) to afford the title compound (0.85 g, 70.2% yield) as a light yellow solid. ¹H NMR (400 MHz, CDCl₃) δ ppm 7.19 (t, J=4.0 Hz, 1H), 6.85 (brs, 1H), 6.74 (s, 1H), 6.57 (dd, J=1.6 Hz, 4.4 Hz, 1H), 3.50-3.41 (m, 4H), 1.94-1.90 (m, 2H), 1.67-1.65 (m, 2H), 1.57-1.55 (m, 2H). LC-MS: [M+H]⁺=360.9, 362.9.

### Step 8: methyl 1-(5-(5-(5-fluorothiophen-2-yl)isoxazole-3-carboxamido)pentyl)azetidine-3-carboxylate

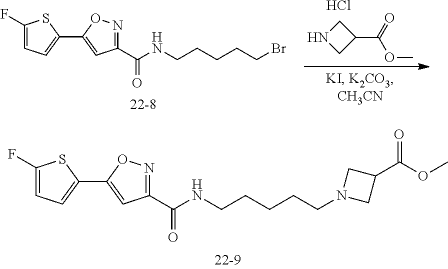

To a suspension of compound 22-8 (400 mg, 1.11 mmol, 1 eq) in CH₃CN (8 mL) was added K₂CO₃ (459 mg, 3.32 mmol, 3 eq) and KI (184 mg, 1.11 mmol, 1 eq) at 0° C. After addition 10 min, compound 22-8a (335 mg, 2.21 mmol, 2.0 eq) was added and stirred at 24-30° C. for 18 h. LCMS showed the reaction was completed. The mixture was filtered. The filtrate was concentrated under reduced pressure. The residue was purified by column chromatography on silica gel (CH₂Cl₂:MeOH=50:1 to 10:1) to afford the title compound (355 mg, 81.07% yield) as a colorless oil. LC-MS: [M+H]⁺=396.1.

### Step 9: 1-(5-(5-(5-fluorothiophen-2-yl)isoxazole-3-carboxamido)pentyl)azetidine-3-carboxylic acid

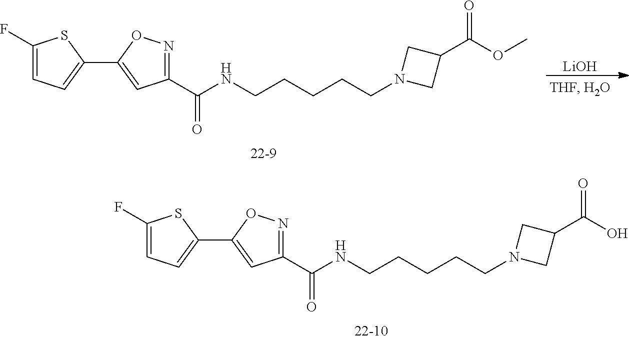

To a solution of compound 22-9 (200 mg, 0.505 mmol, 1.0 eq) in MeOH (5 mL) was added water (2.5 mL) and LiOH.H₂O (64 mg, 1.52 mmol, 3.0 eq) at 0° C. The mixture was stirred at 17-20° C. for 18 hours. LCMS showed the reaction was completed. The mixture was acidified with aq. HCl (1.0 M) to adjust pH to 3-4 and remove the THF under reduced pressure and the residual aqueous was lyophilized to give compound 22-10 (190 mg, 100% yield) as a yellow solid. LC-MS: [M+H]⁺=382.1.

### Step 10: 5-(5-fluorothiophen-2-yl)-N-(5-(3-(methylcarbamoyl)azetidin-1-yl)pentyl)isoxazole-3-carboxamide

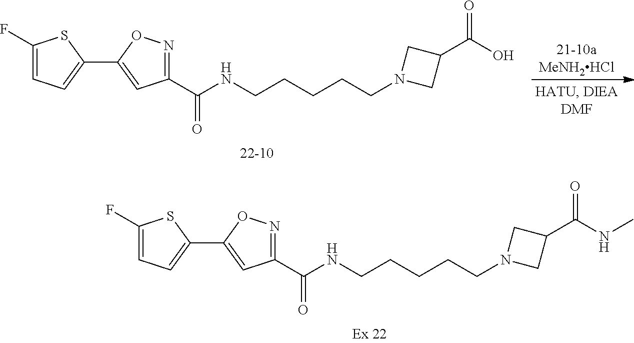 [SMILES: O=C(NCCCCCN1CC(C(=O)O)C1)c1cc(-c2ccc(F)s2)on1 ; CNC(=O)C1CN(CCCCCNC(=O)c2cc(-c3ccc(F)s3)on2)C1]

To a stirred solution of compound 22-10 (180 mg, 0.471 mmol, 1.0 eq) in DMF (2 mL) was added DIEA (305 mg, 2.36 mmol, 5 eq) and compound 22-10a (95.6 mg, 1.42 mmol, 3 eq) at 18-22° C. Then the mixture was stirred at for 3 min, HATU (359 mg, 0.943 mmol, 2 eq) was added. After addition the reaction was stirred at 18-22° C. for 18 hours. LCMS showed the reaction was completed. The mixture was diluted with DMF (3 mL) and purified by prep-HPLC (Kromasil 150*25 mm*10 um, gradient: 25-55% B (A=water (0.05% ammonia hydroxide v/v), B═CH₃CN), flow rate: 25 mL/min) to afford the title compound (40 mg, 21.49% yield) as a yellow solid. 41 NMR (400 MHz, CD₃OD) δ ppm 7.35 (t, J=4.0 Hz, 1H), 6.90 (s, 1H), 6.72 (dd, J=2.8 Hz, 4.4 Hz, 1H), 3.50-3.49 (m, 2H), 3.36-3.34 (m, 2H), 3.29-3.22 (m, 3H), 2.70 (s, 3H), 2.48-2.45 (m, 2H), 1.62-1.59 (m, 2H), 1.38-1.37 (m, 4H). LC-MS: [M+H]⁺=381.1.

### Example 23

### N-(5-(3-carbamoylazetidin-1-yl)pentyl)-5-(5-fluorothiophen-2-yl)isoxazole-3-carboxamide

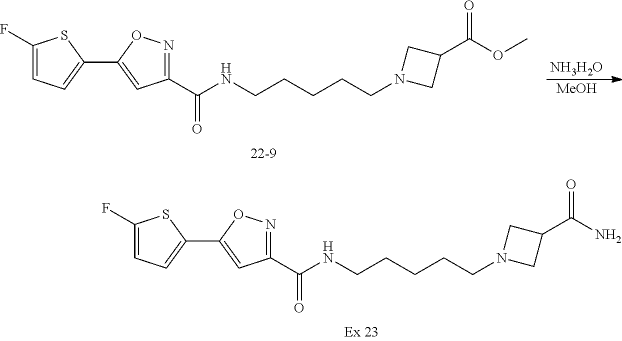

To a solution of compound 21-9 (150 mg, 0.379 mmol, 1.0 eq) in MeOH (1 mL) was added NH₃H₂O (1 mL) at 0° C. Then the mixture was allowed warm to 17-20° C. and stirred for 18 hours. LCMS showed the reaction was completed. The mixture was diluted with MeOH (3 mL) and purified by prep-HPLC (Xtimate C18 150*25 mm*5 um, gradient: 9-39% B (A=water (0.05% ammonia hydroxide v/v), B═CH₃CN), flow rate: 25 mL/min) to afford the title compound (43 mg, 29.8% yield) as a white solid. ¹H NMR (400 MHz, CDCl₃) δ ppm 7.35 (t, J=4.0 Hz, 1H), 6.90 (s, 1H), 6.75 (dd, J=2.0 Hz, 4.4 Hz, 1H), 3.56-3.54 (m, 2H), 3.39-3.33 (m, 2H), 3.29-3.28 (m, 3H), 2.53-2.49 (m, 2H), 1.66-1.63 (m, 2H), 1.42-1.41 (m, 4H). LC-MS: [M+H]⁺=381.1.

### Example 24

### N-(5-(3-((1-cyanoethyl)carbamoyl)azetidin-1-yl)pentyl)-5-(4-fluorophenyl)isoxazole-3-carboxamide

### Step 1: benzyl 3-((l-methoxy-1-oxopropan-2-yl)carbamoyl)azetidine-1-carboxylate

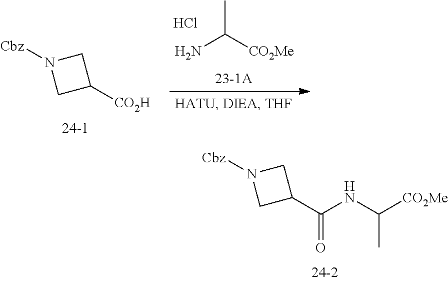

To a solution of compound 24-1 (2.0 g, 8.5 mmol, 1.0 eq) in THF (50 mL) were added compound 24-1A (2.37 g, 17 mmol, 2.0 eq), DIEA (5.49 g, 42.5 mmol, 5.0 eq), HATU (6.47 g, 17 mmol, 2.0 eq) at 4-9° C. The mixture was stirred at 4-9° C. for 14 hours. LCMS showed the reaction was completed. The reaction was quenched by 50 mL of saturated aqueous NaHCO₃. The layers were separated. The aqueous phase was extracted with EtOAc (30 mL*3). The combined organic phase was washed with aqueous HCl (1M, 50 mL) and brine (50 mL). The organic phase was dried over Na₂SO₄, filtered. The filtrate was concentrated under reduced pressure. The residue was purified by column chromatography on silica gel (CH₂Cl₂/MeOH 50:1 to 10:1) to afford the title compound (2.2 g, 86.8% yield) as a light yellow solid. LC-MS: [M+Na]⁺=343.0.

### Step 2: benzyl 3-((1-amino-1-oxopropan-2-yl)carbamoyl)azetidine-1-carboxylate

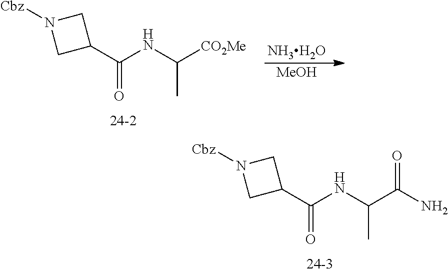 [SMILES: "MeOH" ; COC(=O)C(C)NC(=O)C1CN(C(=O)OCc2ccccc2)C1 ; "NH3·H2O" ; CC(NC(=O)C1CN(C(=O)OCc2ccccc2)C1)C(N)=O]

To a mixture of compound 24-2 (1.0 g, 3.12 mmol, 1.0 eq) in MeOH (15 mL) was added NH₃.H₂O (30 mL, 192.6 mmol, 61.7 eq, 25% wt) at 7-14° C. The mixture was stirred at 2530° C. for 14 hours. LCMS showed the reaction was completed. The volatile was removed under reduced pressure. The residue was purified by acidic pre-HPLC (Agela ASB 150*25 mm*5 um, gradient: 20-40% B (A=water (0.05% TFA v/v), B═CH₃CN), flow rate: 25 mL/min) to afford the title compound (350 mg, 36.7% yield) as a white solid. LC-MS: [M+H]⁺=306.1.

### Step 3: benzyl 3-((1-cyanoethyl)carbamoyl)azetidine-1-carboxy late

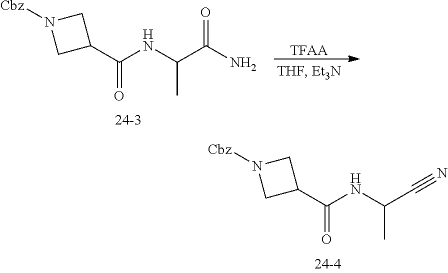 [SMILES: CC(NC(=O)C1CN(C(=O)OCc2ccccc2)C1)C(N)=O ; "TFAA" ; CC(C#N)NC(=O)C1CN(C(=O)OCc2ccccc2)C1]

To a mixture of compound 24-3 (168 mg, 0.55 mmol, 1.0 eq) in THF (2 mL) was added Et₃N (78 mg, 0.77 mmol, 1.4 eq) at 1-8° C. To the mixture was added TFAA (150.2 mg, 0.715 mmol, 1.3 eq) at 1-8° C. The mixture was stirred at 1-8° C. for 4 hours. LCMS showed about 55% of desired product was formed and 25% of starting material was remained. The mixture was quenched with 10 mL of brine. The aqueous was extracted with EtOAc (10 mL*3). The combined organic phase was dried over Na₂SO₄, filtered. The filtrate was concentrated under reduced pressure to afford compound 24-4 (0.3 mmol, crude) as a light yellow solid. LC-MS: [M+Na]⁺=309.9.

### Step 4: N-(1-cyanoethyl)azetidine-3-carboxamide

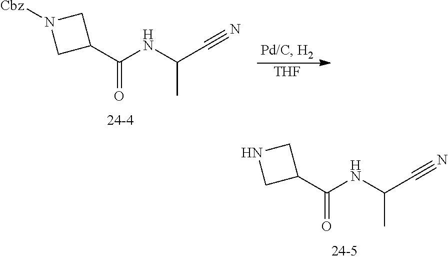 [SMILES: CC(C#N)NC(=O)C1CN(C(=O)OCc2ccccc2)C1 ; CC(C#N)NC(=O)C1CNC1 ; "THF"]

To an autoclave was charged with compound 24-4 (0.3 mmol, crude), Pd/C (50 mg, 10% wt, wet), THF (10 mL) under nitrogen atmosphere. The mixture was stirred at 3-8° C. under hydrogen atmosphere (15 psi) for 4 hours. LCMS showed most of the starting material was consumed. The mixture was filtered, and the cake was washed with THF (2 mL*2). The filtrate was concentrated under reduced pressure to afford compound 24-5 (0.3 mmol) as a colorless oil.

### Step 5: N-(5-(3-((l-cyanoethyl)carbamoyl)azetidin-1-yl)pentyl)-5-(4-fluorophenyl)isoxazole-3-carboxamide

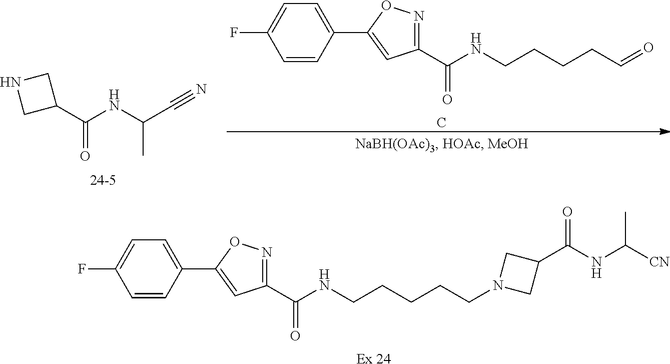

To a mixture of compound 24-5 (0.3 mmol, 1.0 eq) in MeOH (2 mL) was added Intermediate C (100 mg, 0.345 mmol, 1.15 eq), HOAc (18 mg, 0.3 mmol, 1.0 eq), NaBH(OAc)₃ (127.3 mg, 0.6 mmol, 2.0 eq) at 3-9° C. The mixture was stirred at 3-9° C. for 14 hours. LCMS showed the reaction was completed. The reaction was quenched by water (0.5 mL). The mixture was purified by basic pre-HPLC (Kromasil 150*25 mm*10 um, gradient: 33-43% B (A=water (0.05% ammonia hydroxide v/v), B═CH₃CN), flow rate: 30 mL/min) and acidic pre-HPLC (Boston Green ODS 150*30 5 u, gradient: 16-46% B (A=water (0.05% TFA v/v), B═CH₃CN), flow rate: 30 mL/min) respectively to afford Example 24 (18.4 mg, 14.3% yield) as a white solid. ¹H NMR (400 MHz, CD₃OD) δ ppm 7.95-7.91 (m, 2H), 7.31-7.26 (m, 2H), 7.07 (s, 1H), 4.42-4.38 (m, 2H), 4.20-4.05 (m, 2H), 3.70-3.40 (m, 4H), 3.28-3.25 (m, 2H), 1.80-1.64 (m, 4H), 1.53 (d, J=7.6 Hz, 3H), 1.47-1.35 (m, 2H). LC-MS: [M+H]⁺=428.3.

### Example 25

### N-(5-(3-carbamoyl-4-methylpiperazin-1-yl)pentyl)-5-(thiophen-2-yl)isoxazole-3-carboxamide

### Step 1: piperazine-2-carbonitrile

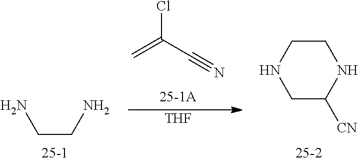

To a mixture of compound 25-1 (5.15 g, 85.71 mmol, 1.5 eq) in THF (15 mL) was added compound 25-1A (5 g, 57.14 mmol, 1.0 eq) drop-wise over 30 min at 0° C. The mixture was stirred for 16 hours at 10-15° C. The mixture was filtered. The filtrate was acidized by 35% HCl to PH=4. The mixture was filter and the residue was dissolved into 20% HCl. The result mixture was poured into THF (300 mL) and filtered to afford the title compound (3.8 g, 57% yield) as a white solid. ¹H NMR: (400 MHz, D₂O) ppm 4.83-4.75 (m, 1H), 3.74-3.52 (m, 2H), 3.49-3.21 (m, 4H).

### Step 2: 4-benzylpiperazine-2-carbonitrile

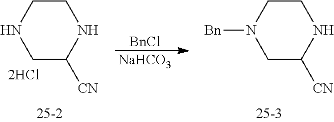

To a mixture of compound 25-2 (3.5 g, 18.89 mmol, 1.0 eq), BnCl (2.39 g, 18.89 mmol, 1.0 eq) and NaHCO₃ (7.14 g, 85.02 mmol, 4.5 eq) in EtOH (150 mL) was heated to 85° C. and stirred for 1 hours at 85° C. The mixture was filtered and the filtrate was concentrated to give a residue. The residue was purified by reversed flash (base) to afford the title compound (1.5 g, 90% purity, 35.5%) as a yellow oil. ¹H NMR (400 MHz, CDCl₃) δ ppm 7.45-7.29 (m, 5H), 3.98 (t, J=3.4 Hz, 1H), 3.67-3.60 (d, 1H), 3.59-3.51 (d, 1H), 3.26-3.24 (m, 1H), 2.92 (m, 1H), 2.88-2.81 (m, 1H), 2.74 (d, J=11.0 Hz, 1H), 2.55-2.42 (m, 1H), 2.41-2.27 (m, 1H), 1.91 (br s, 1H).

Step: 4-benzyl-1-methylpiperazine-2-carbonitrile

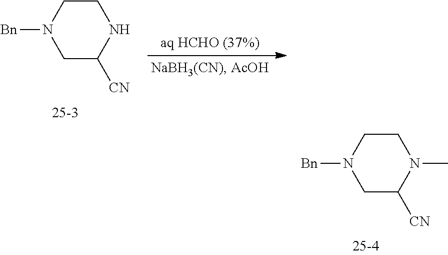 [SMILES: N#CC1CN(Cc2ccccc2)CCN1 ; CN1CCN(Cc2ccccc2)CC1C#N]

To a solution of compound 25-3 (1.4 g, 6.96 mmol, 1.0 eq), aq HCHO (37%, 6 mL) and AcOH (417.72 mg, 6.96 mmol, 1.0 eq in MeOH (30 mL) were added NaBH₃(CN) (874.25 mg, 13.91 mmol, 2.0 eq) at 10° C. The mixture was stirred for 16 h at 10-15. The mixture was concentrated under reduced pressure. The residue was purified by Prep-HPCL (base) to afford the title compound (450 mg, 89% purity, 26.7% yield) as yellow oil. LC-MS: [M+H]⁺=216.3.

### Step 4: 4-benzyl-1-methylpiperazine-2-carboxamide

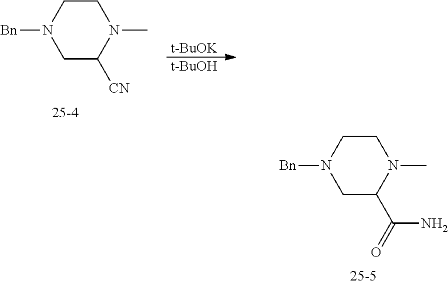 [SMILES: "t-BuOK" ; "t-BuOH" ; CN1CCN(Cc2ccccc2)CC1C#N ; CN1CCN(Cc2ccccc2)CC1C(N)=O]

To a mixture of compound 25-4 (450 mg, 2.09 mmol, 1.0 ea) in t-BuOH (9 mL) was added t-BuOK (938.17 mg, 8.36 mmol, 4.0 eq) at 30° C. The mixture was stirred for 40 h at 30° C. To the mixture was added sat.NH₄Cl (50 mL) and EA (50 mL. The aqueous layer was extracted with EA (50 mL). The combined EA layers were washed with brine (50 mL), dried over Na₂SO₄ and concentrated to afford the title compound (430 mg, crude) as a yellow solid. LC-MS: [M+H]⁺=234.3.

### Step 5: 1-methylpiperazine-2-carboxamide

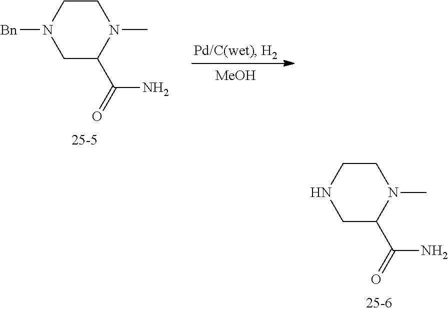

To a mixture of compound 25-5 (230 mg, 985.82 μmol, 1.0 eq) in MeOH (5 mL) was added Pd/C (wet, 10%, 50 mg) at 10° C. The mixture was stirred for 48 hours at 10-15° C. under H2 at 50 psi. TLC showed compound 25-5 was consumed. The mixture was filtered and the filtrate was concentrated under reduced pressure to afford the title compound (140 mg, crude) as a yellow oil.

### Step 6: N-(5-(3-carbamoyl-4-methylpiperazin-1-yl)pentyl)-5-(thiophen-2-yl)isoxazole-3-carboxamide

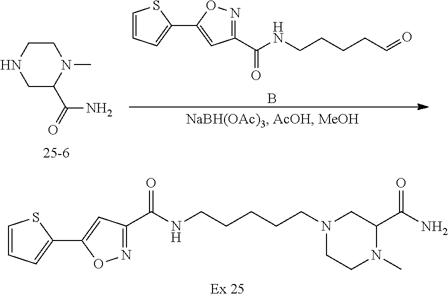

To a mixture of compound 25-6 (140 mg, 977.74 μmol, 1.0 eq) and Intermediate B (244.92 mg, 879.97 μmol, 0.9 eq) in MeOH (5 mL) were added AcOH (58.72 mg, 977.74 μmol, 1.0 eq) and NaBH(OAc)₃ (414.452 mg, 1.96 mmol, 2.0 eq) at 10-15° C. The mixture was stirred for 16 h at 8-15° C. To the mixture was added sat.NaHCO₃ (1 mL). The mixture was filtered. The filtrate was purified by Prep-HPLC (base) (Phenomenex Gemini 150*25 mm*10 um, gradient: 28-58% B (A=water (0.05% ammonia hydroxide v/v), B═CH₃CN), flow rate: 30 mL/min) to afford the title compound (130 mg, 98% purity, 32% yield) as a white solid. ¹H NMR (400 MHz, CD₃OD) δ ppm 7.69 (m, 2H), 7.23 (dd, J=3.8, 5.0 Hz, 1H), 6.93 (s, 1H), 3.41 (t, J=7.0 Hz, 2H), 2.97 (d, J=11.0 Hz, 1H), 2.86 (d, J=9.0 Hz, 2H), 2.75 (dd, J=3.1, 10.4 Hz, 1H), 2.45-2.38 (m, 2H), 2.37-2.29 (m, 1H), 2.29-2.21 (m, 4H), 2.18 (t, J=10.9 Hz, 1H), 1.67 (m, 2H), 1.59 (m, 2H), 1.48-1.37 (m, 2H). LC-MS: [M+H]⁺=406.4.

### Example 26

### N-(5-(4-carbamoyl-4-hydroxypiperidin-1-yl)pentyl)-5-(thiophen-2-yl)isoxazole-3-carboxamide

### Step 1: 1-benzyl-4-hydroxypiperidine-4-carbonitrile

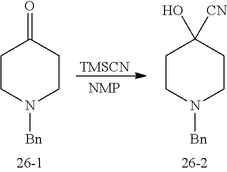 [SMILES: "TMSCN" ; O=C1CCN(Cc2ccccc2)CC1 ; N#CC1(O)CCN(Cc2ccccc2)CC1]

To a mixture of compound 25-1 (4 g, 21.14 mmol, 1.0 eq) in NMP (40 mL) was added TMSCN (4.19 g, 42.27 mmol, 2.0 eq) dropwised at 25° C. The mixture was stirred for 4 hours at 25° C. TLC (PE:EA=10:1, Rf=0.35) showed compound 26-1 was consumed and a new point was appeared. To the mixture was added water (20 mL) and extracted with EA (20 mL*3). The organic layer was dried over Na₂SO₄, filtered and concentrated under reduced pressure to give residue, The residue was purified by column chromatography on silica gel (PE:EA=50:1-20:1) to afford the title compound (2.5 g, 54.7% yield) as a yellow oil.

### Step 2: 1-benzyl-4-hydroxypiperidine-4-carboxamide

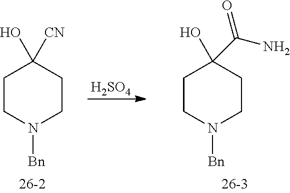

To a mixture of compound 26-2 (2.0 g, 9.25 mmol, 1.0 eq) and in H2SO₄:H₂O (8 mL, v/v=9:1) at 0° C. The mixture reaction was stirred at 25° C. for 16 h. Poured the mixture reaction into water (10 mL) and extracted with EA (15 mL*3). The organic layer was dried over Na₂SO₄, filtered and concentrated under reduced pressure to give residue, The residue was purified by column chromatography on silica gel (PE:EA=2:1-1:1) to afford the title compound (800 mg, pure) as white solid. 41 NMR (400 MHz, CDCl₃) δ ppm 7.36-7.27 (m, 5H), 6.53 (br s, 1H), 5.41 (br s, 1H), 3.55 (s, 2H), 2.85-2.77 (m, 2H), 2.67 (br s, 1H), 2.36-2.25 (m, 2H), 2.24-2.13 (m, 2H), 1.64 (m, 2H). LC-MS: [M+H]⁺=235.1.

### Step 3: 4-hydroxypiperidine-4-carboxamide

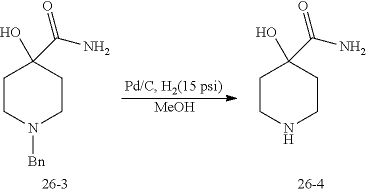 [SMILES: "MeOH" ; NC(=O)C1(O)CCN(Cc2ccccc2)CC1 ; NC(=O)C1(O)CCNCC1]

To a solution of compound 26-3 (650 mg, 2.77 mmol, 1.0 eq) in MeOH (13 mL) was added Pd/C (130 mg) stirred for 2 h at 25° C. TLC (DCM:MeOH=5:1, Rf=0.05) showed compound 26-3 was consumed and a new point was appeared. The mixture reaction was filtered and concentrated in vacuo to the title compound (600 mg, crude) as white solid. ¹H NMR (400 MHz, CD₃OD) δ ppm 2.97-2.83 (m, 4H), 2.05-1.95 (m, 2H), 1.54-1.44 (m, 2H).

### Step 4: N-(5-(4-carbamoyl-4-hydroxypiperidin-1-yl)pentyl)-5-(thiophen-2-yl)isoxazole-3-carboxamide

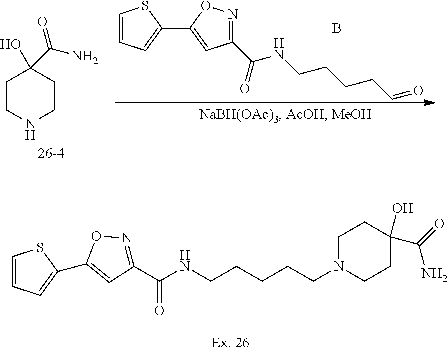

To a solution of compound 26-4 (100 mg, 693.62 umol, 1.0 eq), Intermediate B (96.53 mg, 346.81 umol, 0.5 eq) in MeOH (2 mL) was added HOAc (41.65 mg, 693.62 umol, 1.0 eq) dropwised. Then a solution of NaBH₄ (52.48 mg, 1.39 mmol, 2.0 eq) was added, the mixture reaction was stirred for 2 h at 25° C. TLC (DCM:MeOH=5:1, Rf=0.35) showed Intermediate B was consumed and a new point was appeared. The mixture reaction was added water (5 mL) and extracted with EA (10 mL*3). The combined organic layer was washed with brine (5 mL) dried over Na₂SO₄, filtered and concentrated under reduced pressure to give residue, The residue was purified by Prep-HPLC (base) to afford the title compound (50.27 mg, 99.5% purity) as white solid. ¹H NMR (400 MHz, CD₃OD) δ ppm 7.67 (ddd, J=1.0, 4.4, 9.1 Hz, 2H), 7.21 (dd, J=3.8, 5.0 Hz, 1H), 6.91 (s, 1H), 3.40 (t, J=7.0 Hz, 2H), 2.85-2.80 (m, 2H), 2.48-2.35 (m, 4H), 2.20-2.10 (m, 2H), 1.70-1.55 (m, 6H), 1.46-1.35 (m, 2H). LC-MS: [M+H]⁺=407.3.

### Example 27

### 5-(4-fluorophenyl)-N-(5-(3-(((1S,2R)-2-hydroxycyclopentyl)carbamoyl)azetidin-1-yl)pentyl)isoxazole-3-carboxamide

### Step 1: N-((1S,2S)-2-hydroxycyclopentyl)acetamide (trans relative)

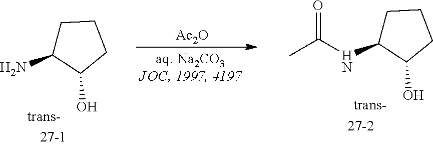

To a starting material compound 27-1 (2.5 g, 24.72 mmol, 1.0 eq) was suspended in 24 mL of acetone at 0° C. and then 24 mL of aqueous 10% Na₂CO₃ was added and followed by addition of Ac₂O (2.52 g 24.72 mmol 1 eq) slowly. Then the reaction was allowed to warm to 20-26° C. over 1 hour and stirred for another 2 hours during which time the solution became homogeneous. The reaction was diluted with 10 mL each of NaHCO₃ (saturated) and NaCl (saturated). The solution was then extracted (5×10 mL) of (CH₂Cl₂:i-PrOH=9:1). The combined organic extracts were dried over Na₂SO₄, filtered, and concentrated to afford the title compound (1.60 g 45.21% yield) as a colorless oil. LC-MS: [M+H]⁺=144.1.

### Step 2: (3aS,6aR)-2-methyl-3a,5,6,6a-tetrahydro-4H-cyclopenta[d]oxazole (cis relative)

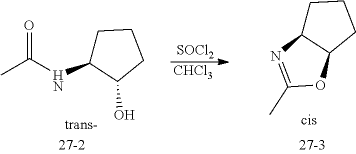

To a stirred solution of compound 27-2 (1.60 g, 11.17 mmol, 1.0 eq) in CH₂Cl₂ (10 mL) was slowly added neat SOCl₂ (5.32 g, 44.7 mmol, 4 eq) at 0° C. The reaction was allowed to warm to 19-26° C. over 1 hour and stirred another 2 hours. The crude mixture was concentrated in vacuo to afford the title compound (1.4 g 100% yield) as a brown oil. ¹H NMR (400 MHz, CD₃OD) δ ppm 5.78-5.75 (m, 1H), 4.87-4.84 (m, 1H), 2.43 (s, 3H), 2.20-2.10 (m, 1H), 2.00-1.87 (m, 4H), 1.70-1.60 (m, 1H).

### Step 3: (1R,2S)-2-aminocyclopentan-1-ol hydrochloride (cis relative)

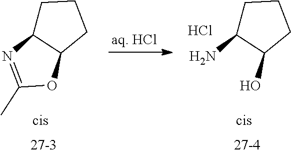

To a solution of compound 27-3 (1.40 g, 9.78 mmol, 1.0 eq) in 15 mL of 1.3N HCl was stirred at 105° C. for 1 hour. The cooled solution was concentrated in vacuo and the resulting residue was dissolved in 1:1 MeOH:CH₂Cl₂ (50 mL) and dried over anhydrous Na₂SO₄, filtered and concentrated to the title compound (500 mg, 40.85% yield) as a brown solid. ¹H NMR (400 MHz, CD₃OD) δ ppm 7.93 (brs, 3H), 5.40 (brs, 1H), 4.09-4.07 (m, 1H), 3.24 (m, 1H), 1.89-1.48 (m, 6H).

### Step 4: 5-(4-fluorophenyl)-N-(5-(3-(((1S,2R)-2-hydroxycyclopentyl)carbamoyl)azetidin-1-yl)pentyl)isoxazole-3-carboxamide

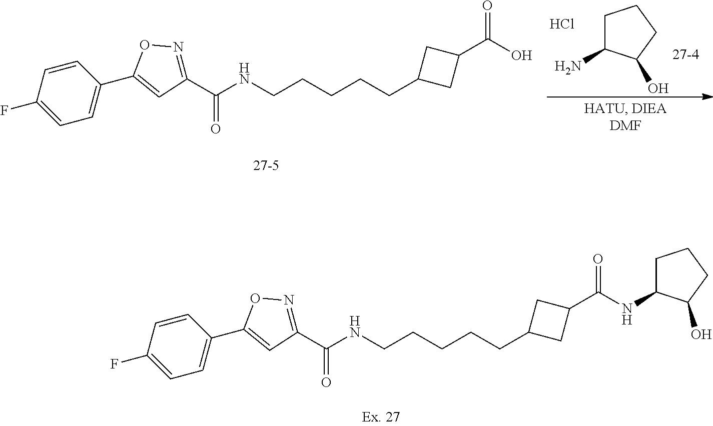 [SMILES: O=C(NCCCCCC1CC(C(=O)O)C1)c1cc(-c2ccc(F)cc2)on1 ; Cl ; N[C@H]1CCC[C@H]1O ; O=C(NCCCCCC1CC(C(=O)N[C@H]2CCC[C@H]2O)C1)c1cc(-c2ccc(F)cc2)on1]

Compound 27-5 was prepared similarly as Compound 22-10 by using a procedure corresponding to Example 22, Steps 6 to 9, but replacing Compound 22-6 with 5-(4-fluorophenyl)isoxazole-3-carboxylic acid. To a stirred solution of Compound 27-5 (200 mg, 0.533 mmol, 1.0 eq) in DMF (3 mL) was added DIEA (344 mg, 2.66 mmol, 5 eq) and Compound 27-4 (219.9 mg, 1.60 mmol, 3 eq) at 18-22° C. Then the mixture was stirred at 18-22° C. for 3 min, HATU (405 mg, 1.07 mmol, 2 eq) was added. After addition the reaction was stirred at 18-22° C. for 18 hours. LCMS showed the reaction was completed. The mixture was diluted with DMF (2 mL) and purified by prep-HPLC (Kromasil 150*25 mm*10 um, gradient: 25-55% B (A=water (0.05% ammonia hydroxide v/v), B ═CH₃CN), flow rate: 25 mL/min) to afford the title compound (89 mg 36.43% yield) as a white solid. LCMS: t_{R}=0.692 min in 5-95AB_220 &254 chromatography (MK RP-18e 25-2 mm), MS (ESI) m/z 459.3 [M+H]⁺. ¹H NMR (400 MHz, CD₃OD) δ ppm 7.93-7.89 (m, 2H), 7.28-7.24 (m, 2H), 7.03 (s, 1H), 4.10-4.07 (m, 1H), 3.97-3.94 (m, 1H), 3.54-3.52 (m, 2H), 3.43-3.39 (m, 2H), 3.37-3.35 (m, 1H), 3.30-3.28 (m, 2H), 2.48-2.47 (m, 2H), 1.87-1.82 (m, 3H), 1.64-1.60 (m, 5H), 1.40-1.38 (m, 4H). LC-MS: [M+H]⁺=459.3.

### Example 28

### N-(5-((3R,4S)-4-carbamoyl-3-fluoropiperidin-1-yl)pentyl)-5-(thiophen-2-yl)isoxazole-3-carboxamide (Ex 28-cis)

### N-(5-((3S,4S)-4-carbamoyl-3-fluoropiperidin-1-yl)pentyl)-5-(thiophen-2-yl)isoxazole-3-carboxamide (Ex 28-trans)

### Step 1: methyl 1-benzyl-3-fluoropiperidine-4-carboxylate

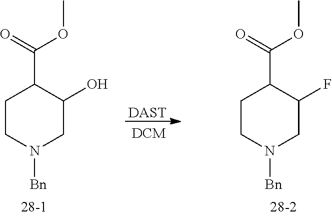

To a mixture of compound 28-1 (5 g, 20.06 mmol, 1.0 eq) in DCM (100 mL) was added DAST (8.08, 50.14 mmol, 2.5 eq) at −60° C. The mixture was allowed to warm to 0° C. and stirred for 3 h. The mixture was stirred for 16 h at 15° C. To sat.NaHCO₃ (250 mL) was added the mixture. The DCM layer was washed with brine (100 mL). The organic layer was dried over Na₂SO₄, filtered and concentrated to afford a residue, which was purified by column chromatography on silica gel (PE:EA=50:1 to 20:1) to the title compound (1.1 g, 94% purity, 20% yield) as a yellow oil.

LC-MS: [M+H]⁺=252.3.

### Step 2: 1-benzyl-3-fluoropiperidine-4-carboxamide

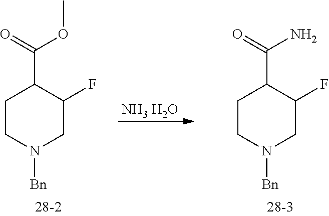

A mixture of compound 28-2 (1.1 g, 4.38 mmol, 1.0 eq) and NH3.H2O (25%, 200 mL) in MeOH (20 mL) was stirred 26 h at 15° C. The mixture was extracted with EA (100 mL×2). The EA layers was washed with brine (100 mL), dried over Na2SO4 and concentrated to give a residue, which was purified by column chromatography on silica gel (PE:EA=1:1) to afford the title compound (340 mg, 34% yield) as a yellow solid. LC-MS: [M+H]⁺=237.2.

### Step 3: 3-fluoropiperidine-4-carboxamide

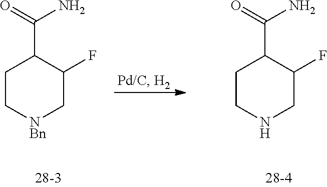

A mixture of compound 28-3 (340 mg, 1.44 mmol, 1.0 eq) and Pd/C (150 mg, 10%, wet) in MeOH (6 mL) was stirred under H2 at 50 psi for 6 h at 15° C. The mixture was filtered and concentrated to the title compound (170 mg, crude) as a yellow oil. ¹H NMR (400 MHz, CD₃OD) δ ppm 3.58-3.46 (m, 1H), 3.11-3.03 (m, 1H), 2.97-2.90 (m, 1H), 2.76-2.66 (m, 1H), 2.61-2.46 (m, 2H), 2.12 (m, 1H), 2.05-1.95 (m, 1H).

### Step 4: N-(5-(4-carbamoyl-3-fluoropiperidin-1-yl)pentyl)-5-(thiophen-2-yl)isoxazole-3-carboxamide

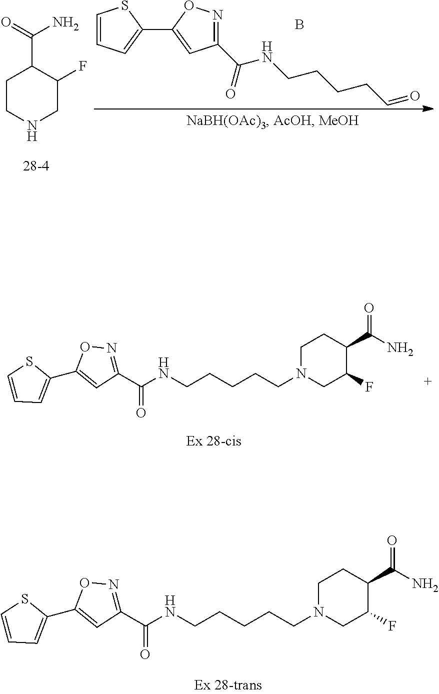

To a mixture of compound 28-4 (160 mg, 1.09 mmol, 1.0 eq) and Intermediate B (274.21 mg, 985.2 μmol, 0.9 eq) in MeOH (5 mL) were added AcOH (65.74, 1.09 mmol, 1.0 eq) and NaBH(OAc)₃ (464.01 mg, 2.19 mmol, 2.0 eq) at 20° C. The mixture was stirred for 4 h at 20° C. To the reaction was added H₂O (1 mL). The mixture was filtered. The filtrate was purified by basic pre-HPLC (Phenomenex Gemini 150*25 mm*10 um, gradient: 25-55% B (A=water (0.05% ammonia hydroxide v/v), B═CH₃CN), flow rate: 30 mL/min) to afford Example 28-cis (71.74 mg, 99% purity) and Example 28-trans (39.32 mg, 98.6% purity) both as a white solid. Example 28-cis and Example 28-trans were both confirmed by LCMS, SFC and HNMR.

Example 28-cis: ¹H NMR (400 MHz, CD₃OD) δ ppm 7.72-7.65 (m, 2H), 7.22 (dd, J=3.8, 5.0 Hz, 1H), 6.92 (s, 1H), 4.55-4.50 (m, 0.5H), 4.43-4.38 (m, 0.5H), 3.46-3.37 (m, 2H), 3.20-3.12 (m, 1H), 3.04-2.85 (m, 2H), 2.85-2.74 (m, 1H), 2.55-2.44 (m, 1H), 2.44-2.18 (m, 2H), 2.14-1.92 (m, 2H), 1.81-1.53 (m, 4H), 1.51-1.36 (m, 2H). LC-MS: [M+H]⁺=409.3.

Example 28-trans: ¹H NMR (400 MHz, CD₃OD) δ ppm 8.80 (br t, J=5.7 Hz, 1H), 7.87 (dd, J=1.1, 5.0 Hz, 1H), 7.80 (dd, J=1.2, 3.7 Hz, 1H), 7.46 (br s, 1H), 7.27 (dd, J=3.7, 4.9 Hz, 1H), 7.18 (s, 1H), 6.95 (br s, 1H), 4.70 (m, 1H), 4.58 (m, 1H), 3.25 (q, J=6.7 Hz, 2H), 3.20-3.10 (m, 1H), 2.78 (d, J=8.8 Hz, 1H), 2.39-2.18 (m, 3H), 1.95-1.69 (m, 3H), 1.61-1.37 (m, 5H), 1.36-1.19 (m, 2H). LC-MS: [M+H]⁺=409.3.

### Example 29

### N-(5-((3R,4S)-3-carbamoyl-4-cyanopiperidin-1-yl)pentyl)-5-(thiophen-2-yl)isoxazole-3-carboxamide

### Step 1: methyl 1-benzyl-4-oxopiperidine-3-carboxylate

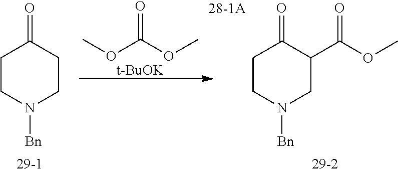

To a solution of compound 29-1A (19.04 g, 211.36 mmol), t-BuOK (29.65 g, 264.20 mmol) in Toluene (250 mL) was added compound 29-1 (20 g, 105.68 mmol) at 90° C. and stirred at 90° C. for 2 hours. TLC (PE:EA=2:1, Rf=0.7) showed compound 29-1 was consumed. The mixture was filtered, the organic layers was diluted with NH₄Cl (aq) (300 mL), extracted with EA (100 mL*3) and concentrated in vacuo to afford the title compound (30 g, crude), as yellow oil. LC-MS: [M+H]⁺=248.3.

### Step 2: methyl 1-benzyl-4-hydroxypiperidine-3-carboxylate

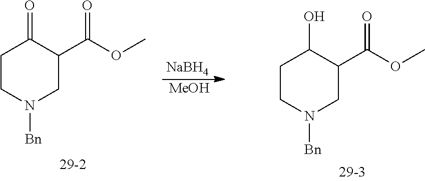

To a solution of compound 28-2 (30 g, 121.32 mmol) in MeOH (300 mL) was added NaBH₄ (6.88 g, 181.97 mmol) at 0° C. and stirred 0° C. for 1 hours. TLC (PE:EA=2:1, Rf=0.1) indicated compound 29-2 was consumed. The mixture was quenched by H₂O (100 mL), concentrated in vacuo to remove MeOH, extracted with EA (100 mL*3) and concentrated in vacuo to afford the title compound (16 g, crude). LC-MS: [M+H]⁺=250.4.

### Step 3: methyl 1-benzyl-4-((methylsulfonyl)oxy)piperidine-3-carboxylate

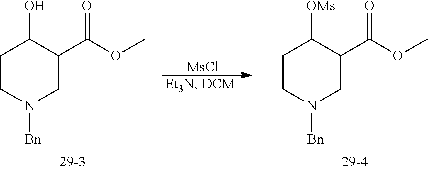

To a solution of compound 28-3 (15 g, 60.17 mmol) in DCM (150 mL) was added Et₃N (24.35 g, 240.67 mmol), MsCl (13.78 g, 120.33 mmol) at 0° C. and stirred at 25° C. for 16 hours. TLC (PE:EA=2:1, Rf=0.6) indicated compound 29-3 was consumed, the mixture was washed with NH₄Cl (aq) (100 mL*5) and concentrated in vacuo to give compound 29-4 (20 g, crude) as yellow oil, which was used to the next step directly.

### Step 4: methyl (3 S,4R)-1-benzyl-4-cyanopiperidine-3-carboxy late

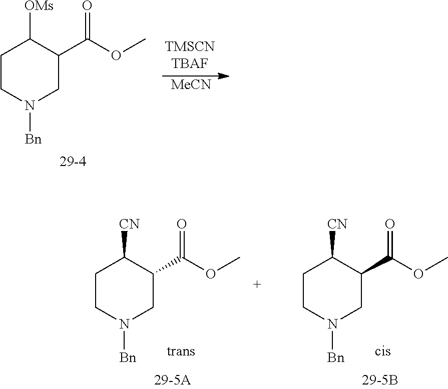 [SMILES: "MeCN" ; COC(=O)C1CN(Cc2ccccc2)CCC1OS(C)(=O)=O ; COC(=O)[C@@H]1CN(Cc2ccccc2)CC[C@H]1C#N ; COC(=O)[C@H]1CN(Cc2ccccc2)CC[C@H]1C#N]

To a solution of compound 29-4 (20 g, 61.09 mmol) in MeCN (200 mL) was added TBAF (91.63 mL, 91.63 mmol), TMSCN (9.09 g, 91.63 mmol) and stirred at 80° C. for 16 hours. TLC (PE:EA=2:1, Rf=0.6, 0.4) indicated compound 29-4 was consumed, the mixture was concentrated in vacuo to give a residue, then the residue was diluted with H₂O (150 mL), extracted with EA (100 mL*3), concentrated in vacuo to give a residue (12 g, crude). The residue was purified by column chromatography (PE:EA=100:1 to 10:1) to give compound 29-5A (3.0 g, 16.9% yield, 89% purity) and compound 29-5B (2.5 g, 13.4% yield, 85% purity).

Compound 29-5A: ¹H NMR (400 MHz, CD₃OD) δ ppm 7.42-7.20 (m, 5H), 3.70 (s, 3H), 3.58-3.47 (m, 2H), 3.02 (d, J=3.3 Hz, 1H), 2.97-2.82 (m, 2H), 2.81-2.67 (m, 1H), 2.45-2.19 (m, 2H), 2.17-2.05 (m, 1H), 1.89-1.75 (m, 1H). LC-MS: [M+H]⁺=259.2.

Compound 29-5B: ¹H NMR (400 MHz, CD₃OD) δ ppm 7.38-7.21 (m, 5H), 3.71 (s, 3H), 3.62-3.49 (m, 2H), 3.37 (m, 1H), 3.10-2.88 (m, 2H), 2.81-2.64 (m, 1H), 2.57-2.26 (m, 2H), 2.12-1.99 (m, 1H), 1.95-1.83 (m, 1H). LC-MS: [M+H]⁺=259.2.

### Step 5: (3 S,4R)-1-benzyl-4-cyanopiperidine-3-carboxamide

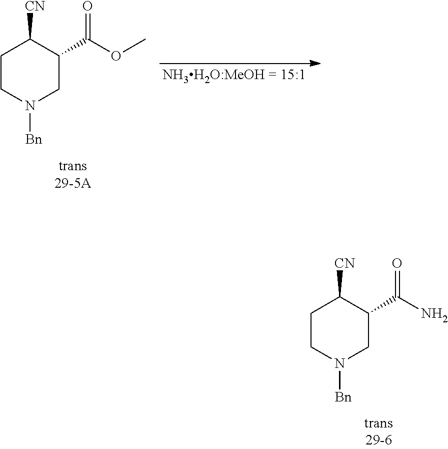 [SMILES: COC(=O)[C@@H]1CN(Cc2ccccc2)CC[C@H]1C#N ; N#C[C@@H]1CCN(Cc2ccccc2)C[C@H]1C(N)=O]

A solution of compound 29-5A (2.0 g, 7.74 mmol) in MeOH (2 mL) and NH₃.H₂O (20 mL, 25% purity) was stirred at 25° C. for 16 hours. TLC (PE:EA=2:1, Rf=0.4) indicated compound 29-5A was consumed, the mixture was extracted with EA (15 mL*5), concentrated in vacuo to give a residue. The residue was purified by column chromatography (PE:EA=10:1 to 0:1) to afford the title compound (750 mg, 39.81% yield). LC-MS: [M+H]⁺=244.3.

### Step 6: (3 S,4R)-4-cyanopiperidine-3-carboxamide

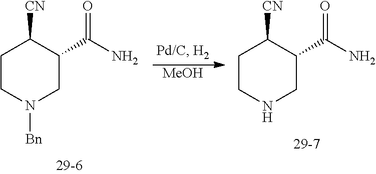 [SMILES: "MeOH" ; N#C[C@@H]1CCN(Cc2ccccc2)C[C@H]1C(N)=O ; N#C[C@@H]1CCNC[C@H]1C(N)=O]

To a solution of compound 29-6 (200 mg, 0.822 mmol) in MeOH (2 mL) was added Pd/C (200 mg, 10% purity) and stirred under H2 (15 Psi) at 25° C. for 5 hours. TLC (DCM:MeOH=10:1, Rf=0.2) indicated compound 29-6 was consumed, the mixture was filtered and concentrated in vacuo to afford the title compound (120 mg, crude), which was used to the next step directly.

### Step 7: N-(5-((3R,4S)-3-carbamoyl-4-cyanopiperidin-1-yl)pentyl)-5-(thiophen-2-yl)isoxazole-3-carboxamide

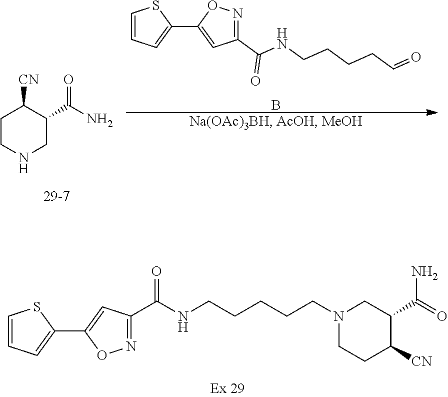

To a solution of compound 29-7 (120 mg, 0.783 mmol) in MeOH (2 mL) was added Intermediate B (218.04 mg, 0.783 mmol), AcOH (47.04 mg, 0.783 mmol), NaBH(OAc)₃ (332.06 mg, 1.57 mmol) and stirred at 25° C. for 16 hours. LCMS showed 38.7% of Intermediate B was remain, 21% of Example 29 was detected. The mixture was concentrated in vacuo to give a residue. The residue was purified by prep-HPLC (TFA condition) to give a residue, the residue was diluted with NaHCO₃aq (10 mL) extracted with EA (10 mL*3), the organic layers was concentrated in vacuo to afford the title compound (53.45 mg, 16.42% yield). 41 NMR (400 MHz, CD₃OD) δ ppm 7.71-7.63 (m, 2H), 7.21 (t, J=4.5 Hz, 1H), 6.90 (s, 1H), 3.39 (t, J=7.0 Hz, 2H), 3.05 (dd, J=11.7 Hz, 1H), 2.97-2.82 (m, 2H), 2.72 (dt, J=3.7, 10.7 Hz, 1H), 2.44-2.36 (m, 2H), 2.16-2.02 (m, 3H), 1.88-1.76 (m, 1H), 1.65 (m, 2H), 1.61-1.51 (m, 2H), 1.46-1.36 (m, 2H). LC-MS: [M+H]⁺=416.3.

### Example 30

### N-(5-(4-carbamoyl-3-hydroxypiperidin-1-yl)pentyl)-5-(thiophen-2-yl)isoxazole-3-carboxamide

### Step 1: methyl 1-benzyl-3-oxopiperidine-4-carboxylate

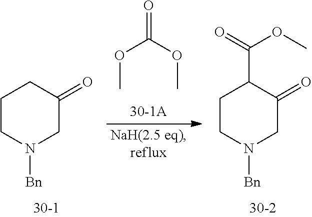

To a solution of Compound 30-1 (14 g, 74 mmol, 1 eq) in 30-1A (100 mL) was added NaH (7.6 g, 190 mmol, 2.5 eq) at 0° C., the reaction mixture was stirred at 75° C. for 1 h. TLC (PE:EA=1:1) showed the most of staring material (Rf=0.7) was consumed and one new spot (Rf=0.8, the same to C-05663-028-P1) was observed. The reaction mixture was diluted with water (200 mL) and extracted with EA (30 mL*3). The combined organic layer was washed with brine (20 mL), dried over Na₂SO₄, filtered and concentrated to afford the title compound (15 g, crude, 80% purity), it was obtained as dark oil and used for next step directly. 41 NMR (400 MHz, CDCl₃) δ ppm 7.38-7.28 (m, 5H), 3.78 (s, 3H), 3.61 (s, 2H), 3.13 (s, 2H), 2.61 (t, J=5.6 Hz, 2H), 2.37-2.33 (m, 2H).

### Step 2: methyl 1-benzyl-3-hydroxypiperidine-4-carboxylate

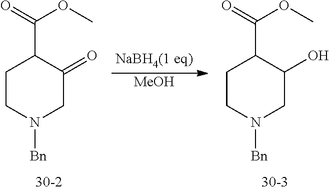 [SMILES: "MeOH" ; COC(=O)C1CCN(Cc2ccccc2)CC1=O ; COC(=O)C1CCN(Cc2ccccc2)CC1O]

To a solution of Compound 30-2 (3 g, 12 mmol, 1.0 eq) in MeOH (20 mL) was added NaBH₄ (229 mg, 6 mmol, 0.5 eq) at 0° C. The reaction mixture was stirred at 15° C. for 1 h. TLC (PE:EA=1:1) showed the most of staring material (Rf=0.8) was consumed and one new spot (Rf=0.4, the same to C-05663-039-P1) was observed. The reaction mixture was quenched by 2M HCl to adjust pH=7 and then extracted with EA (20 mL*3). The combined organic phase was dried over Na₂SO₄, filtered and concentrated to afford the title compound (2.6 g, crude, 80% purity), it was obtained as dark oil and used for next step directly. ¹H NMR (400 MHz, CDCl₃) δ ppm 7.34-7.29 (m, 5H), 3.74 (s, 3H), 3.56 (s, 2H), 3.04-2.96 (m, 1H), 2.90 (m, 1H), 2.47-2.38 (m, 1H), 2.28-2.19 (m, 1H), 2.15-1.88 (m, 2H), 1.84-1.72 (m, 2H).

### Step 3: 1-benzyl-3-hydroxypiperidine-4-carboxamide

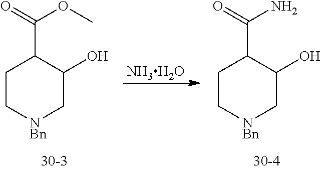 [SMILES: COC(=O)C1CCN(Cc2ccccc2)CC1O ; "NH3·H2O" ; NC(=O)C1CCN(Cc2ccccc2)CC1O]

To a mixture of Compound 30-3 (2 g, 8 mmol, 1 eq) in MeOH (1 mL) was added NH3.H2O (20 mL). The reaction mixture was stirred at 15° C. for 16 hrs. TLC (DCM:MeOH=10:1) showed the most of staring material (Rf=0.6) was consumed and one new spot (Rf=0.25, the same to C-05665-022-P1) was observed. The reaction mixture was concentrated in vacuo, the residue was diluted with EA (10 mL) and washed with brine (50 mL). The combined organic phase was dried over Na₂SO₄, filtered and concentrated to afford the title compound (1.3 g, crude, 85% purity) as yellow oil, it was used for next step directly. LC-MS: [M+H]⁺=235.4.

### Step 4: 3-hydroxypiperidine-4-carboxamide

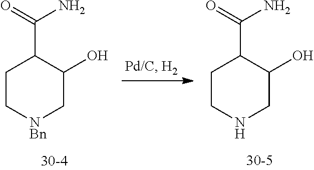 [SMILES: NC(=O)C1CCN(Cc2ccccc2)CC1O ; NC(=O)C1CCNCC1O]

To a mixture of Compound 30-4 (1 g, 4.27 mmol, 1.0 eq) in MeOH (8 mL) was added Pd/C (0.6 g, 10%), The reaction mixture was stirred under H2 (50 Psi) at 15° C. for 6 hrs. TLC (DCM:MeOH=10:1) showed the most of staring material (Rf=0.25) was consumed and one new spot (Rf=0.01, the same to C-05663-085-P1) was observed. The reaction mixture was filtered and the organic phase was concentrated in vacuo to afford the title compound (350 mg, crude, 85% purity) as yellow oil, it used for next step directly.

### Step 5

### N-(5-((3R,4R)-4-carbamoyl-3-hydroxypiperidin-1-yl)pentyl)-5-(thiophen-2-yl)isoxazole-3-carboxamide (cis)

### N-(5-((3S,4R)-4-carbamoyl-3-hydroxypiperidin-1-yl)pentyl)-5-(thiophen-2-yl)isoxazole-3-carboxamide (trans)

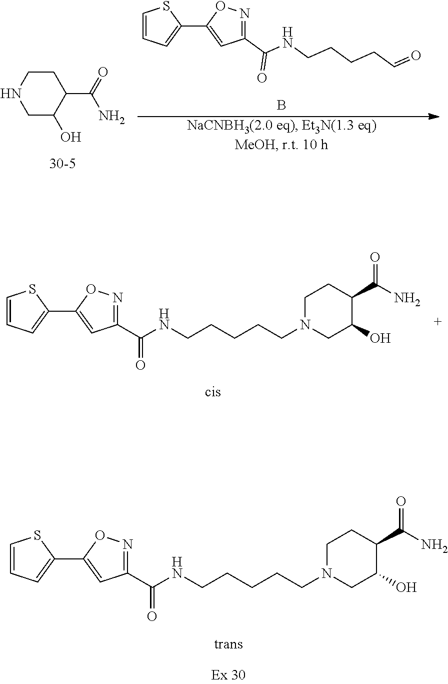

To a mixture of Compound 30-5 (300 mg, 2.08 mmol, 1.0 eq) in MeOH (8 mL) was added Intermediate B (579 mg, 2.08 mmol, 1.0 eq) and HOAc (125 mg, 2.08 mmol, 1.0 eq), followed by additional of NaBH(OAc)₃ (881 mg, 4.16 mmol, 2.0 eq) at 15° C. The reaction mixture was stirred at 15° C. for 6 hrs. LCMS (C-05665-047-P1A2) showed Compound 30-5 was consumed completely, desired MW was observed as main peak. The reaction mixture was quenched by saturated aqueous NaHCO₃ (100 mL) and extracted with EA (20 mL*3). The organic phase was dried over Na₂SO₄, filtered and concentrated. The residue was purified by Pre-HPLC(NH₃.H₂O) to afford the title compounds (80 mg cis, 16 mg trans) as white solid.

Example 30 (cis): ¹H NMR (400 MHz, CDCl₃) δ ppm 8.80 (m, 1H), 7.88 (dd, 5.2 Hz, 1H), 7.80 (dd, J=1.2, 3.6 Hz, 1H), 7.28 (dd, 5.2 Hz, 1H), 7.21 (s, 1H), 7.18 (s, 1H), 6.87 (s, 1H), 4.55 (d, J=5.6 Hz, 1H), 3.93 (s, 1H), 3.25 (m, 2H), 2.69-2.68 (m, 1H), 2.26-2.22 (m, 2H), 2.18-2.16 (m, 1H), 2.09-2.05 (m, 1H), 1.97-1.87 (m, 2H), 1.58-1.41 (m, 5H), 1.34-1.22 (m, 2H). LC-MS: [M+H]⁺=407.3.

Example 30 (trans): ¹H NMR (400 MHz, DMSO-d₆) δ ppm 8.81 (m, 1H), 7.88 (dd, J=1.2, 5.2 Hz, 1H), 7.80 (dd, J=1.2, 3.6 Hz, 1H), 7.28 (dd, J=3.6, 5.2 Hz, 1H), 7.21 (s, 1H), 7.18 (s, 1H), 6.73 (s, 1H), 4.79 (d, J=4.4 Hz, 1H), 3.29-3.21 (m, 3H), 3.18 (d, 1H), 2.90 (m, 1H), 2.78 (m, 1H), 2.27-2.21 (m, 2H), 1.95-1.85 (m, 1H), 1.76-1.62 (m, 2H), 1.60-1.53 (m, 2H), 1.50-1.43 (m, 2H), 1.41-1.25 (m, 2H). LC-MS: [M+H]⁺=407.3.

### Example 31

### N-(5-(4-carbamoyl-3-hydroxypiperidin-1-yl)pentyl)-5-(4-fluorophenyl)isoxazole-3-carboxamide

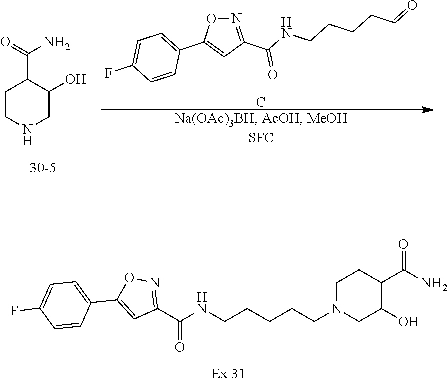

To a solution of Compound 30-5 (140 mg, 0.971 mmol, 1 eq) in MeOH (3 mL) was added Intermediate C (281.89 Mg, 0.971 mmol, 1 eq), AcOH (58.31 mg, 0.971 mmol, 1 eq) and NaBH(OAc)₃ (411.62 mg, 1.94 mmol, 2 eq). The mixture was stirred at 25° C. for 16 hours. LCMS(C-05664-110-P1A) showed 27% of 3 A wan remain, 66% of 4 was detected. The reaction was filtered and concentrated under reduced pressure. The residue was purified by Prep-HPLC (basic condition) to afford the related racemates of title compound (250 mg, 61.5% yield) as a yellow solid separated by SFC.

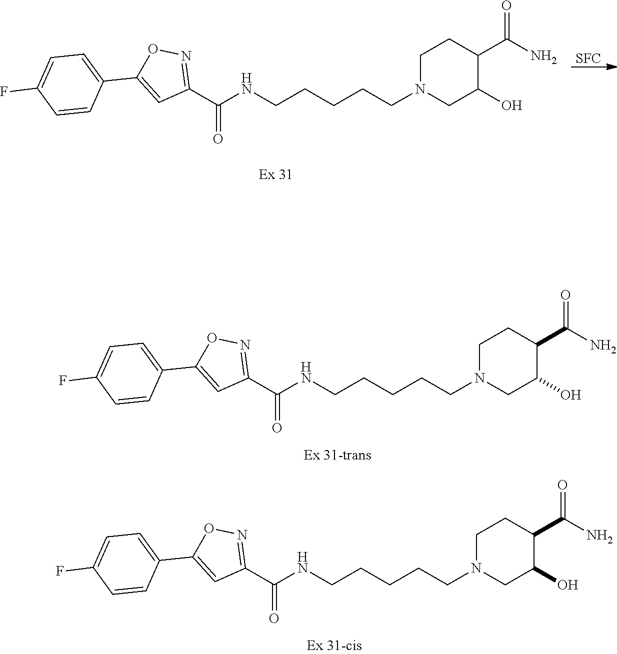

Example 31 was further purified by SFC (column: IC 250 mm*30 mm, 10 um, condition: 0.1% NH₃H₂O MeOH, flow rate: 70 mL/min) to afford Example 31-cis and -trans.

Example 31-cis was obtained as a yellow solid (126.17 mg 96.78% purity 48.84% yield). Example 30-cis was peak 2 in IC and Example 31-trans was peak 2 in IC. Peak 1: ¹H NMR (400 MHz, CD₃OD) δ ppm 8.47 (s, 1H), 7.92 (dd, J=5.3, 8.8 Hz, 2H), 7.28 (t, J=8.7 Hz, 2H), 7.06 (s, 1H), 4.03 (s, 1H), 3.51-3.35 (m, 4H), 3.04-2.92 (m, 2H), 2.88-2.58 (m, 2H), 2.42 (s, 1H), 2.11 (d, J=13.2 Hz, 1H), 1.98-1.83 (m, 1H), 1.81-1.64 (m, 3H), 1.73-1.64 (m, 1H), 1.46 (m, 2H). LC-MS: [M+H]⁺=419.4.

Peak 2: ¹H NMR (400 MHz, CD₃OD) δ ppm 7.96-7.89 (m, 2H), 7.28 (t, J=8.7 Hz, 2H), 7.05 (s, 1H), 4.10 (br s, 1H), 3.40 (t, J=7.0 Hz, 2H), 3.02-2.87 (m, 2H), 2.50-2.33 (m, 3H), 2.28 (br d, J=11.6 Hz, 1H), 2.23-2.06 (m, 2H), 1.71-1.62 (m, 3H), 1.62-1.54 (m, 2H), 1.42 (m, 2H). LC-MS: [M+H]⁺=419.4.

### Example 32

### N-(5-(3-carbamoyl-4-hydroxypyrrolidin-1-yl)pentyl)-5-(4-fluorophenyl)isoxazole-3-carboxamide

### Step 1: tert-butyl 3-cyano-4-hydroxypyrrolidine-1-carboxylate

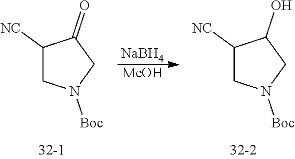 [SMILES: "MeOH" ; CC(C)(C)OC(=O)N1CC(=O)C(C#N)C1 ; "NaBH4" ; CC(C)(C)OC(=O)N1CC(O)C(C#N)C1]

To a mixture of compound 32-1 (3 g, 14.27 mmol, 1.0 eq) in EtOH (60 mL) was NaBH₄ (1.08 g, 28.54 mmol, 2.0 eq) at 0° C. and stirred for 1 h. The mixture was concentrated. The residue was dissolved into EA (50 mL). The EA layer was washed with water (50 mL) and brine (100 mL). The organic layer was dried over Na₂SO₄, filtered and concentrated to afford the title compound (3.3 g, crude) as a yellow oil. LC-MS: [M+H−56]⁺=157.1.

### Step 2: tert-butyl 3-carbamoyl-4-hydroxypyrrolidine-1-carboxylate

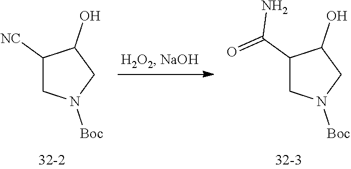 [SMILES: CC(C)(C)OC(=O)N1CC(O)C(C#N)C1 ; CC(C)(C)OC(=O)N1CC(O)C(C(N)=O)C1]

To a solution of compound 32-2 (1.5 g, 7.07 mmol, 1.0 eq) in MeOH (30 mL) were added aq NaOH (15 mL, 1M) and H₂O₂ (7.5 mL) at 15° C. and stirred for 6 h. To the mixture was added sat.NH₄Cl (200 mL) and EA (150 mL). The aqueous layer was extracted with EA (150 mL). The combined EA layers was washed with brine (100 mL), dried over Na₂SO₄ and concentrated under reduced pressure to give the title compound (800 mg, crude) as a yellow oil, which was used for next step directly. LC-MS: [M+Na]⁺=253.1.

### Step 3: 4-hydroxypyrrolidine-3-carboxamide

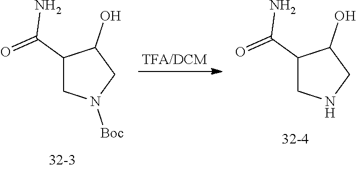 [SMILES: CC(C)(C)OC(=O)N1CC(O)C(C(N)=O)C1 ; NC(=O)C1CNCC1O]

To a mixture of compound 32-3 (800 mg, 3.47 mmol, 1.0 eq) in DCM (15 mL) was added TFA (5 mL) at 15° C. and stirred for 6 h. The mixture was concentrated to give Compound 32-4 (1.6 g, crude) as a yellow oil, which was used for next step directly. LC-MS: [M+H]⁺=131.1.

### Step 4

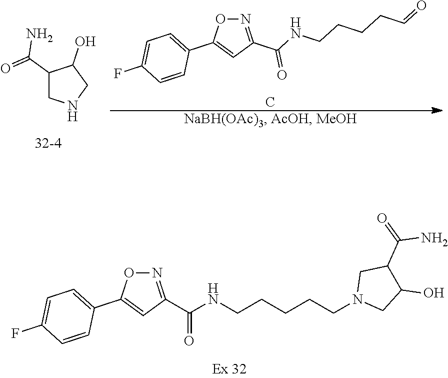

To a mixture of compound 32-4 (800 mg, crude) and Intermediate C (446.1 mg, 1.54 mmol, 1.0 eq) in MeOH (15 mL) were added AcOH (92.28 mg, 1.54 mmol, 1.0 eq) and NaBH(OAc)₃ (651.4 mg, 3.07 mmol, 2.0 eq) at 15° C. The mixture was stirred for 16 h at 15° C. To the reaction was added sat.NaHCO₃ (1.5 mL). The mixture was filtered and the filtrate was purified by basic pre-HPLC (Phenomenex Gemini 150*25 mm*10 um, gradient: 22-52% B (A=water (0.05% ammonia hydroxide v/v), B═CH₃CN), flow rate: 30 mL/min) and twice SFC to afford four peaks of Example 32 all as a white solid.

Example 32 (peak 1): ¹H NMR (400 MHz, CD₃OD) δ ppm 8.00-7.89 (m, 2H), 7.34-7.25 (m, 2H), 7.07 (s, 1H), 4.57-4.45 (m, 1H), 3.42 (t, J=7.0 Hz, 2H), 3.17-3.07 (m, 1H), 3.06-2.92 (m, 3H), 2.66-2.52 (m, 3H), 1.76-1.55 (m, 4H), 1.51-1.36 (m, 2H). LC-MS: [M+H]⁺=405.3.

Example 32 (peak 2): ¹H NMR (400 MHz, CD₃OD) δ ppm 7.93-7.73 (m, 2H), 7.27-7.10 (m, 2H), 6.95 (s, 1H), 4.46-4.32 (m, 1H), 3.31 (t, J=7.0 Hz, 2H), 3.08-2.85 (m, 4H), 2.61-2.49 (m, 3H), 1.63-1.44 (m, 4H), 1.41-1.27 (m, 2H). LC-MS: [M+H]⁺=405.3.

Example 32 (peak 3): ¹H NMR (400 MHz, CD₃OD) δ ppm 8.01-7.84 (m, 2H), 7.29 (t, J=8.8 Hz, 2H), 7.07 (s, 1H), 4.46 (q, J=4.9 Hz, 1H), 3.42 (t, J=7.1 Hz, 2H), 3.10 (t, J=8.8 Hz, 1H), 2.85 (m, 1H), 2.75 (d, J=5.1 Hz, 2H), 2.65-2.37 (m, 3H), 1.74-1.52 (m, 4H), 1.52-1.39 (m, 2H). LC-MS: [M+H]⁺=405.3.

Example 32 (peak 4): ¹H NMR (400 MHz, CD₃OD) δ ppm 8.01-7.84 (m, 2H), 7.39-7.20 (m, 2H), 7.07 (s, 1H), 4.47 (q, J=4.7 Hz, 1H), 3.42 (t, J=7.0 Hz, 2H), 3.16 (dd, J=8.3, 9.5 Hz, 1H), 2.88 (m, 1H), 2.81 (d, J=4.9 Hz, 2H), 2.74-2.47 (m, 3H), 1.74-1.54 (m, 4H), 1.52-1.36 (m, 2H). LC-MS: [M+H]⁺=405.3.

### Example 33

### N-(5-(3-carbamoyl-4-hydroxypyrrolidin-1-yl)pentyl)-5-(thiophen-2-yl)isoxazole-3-carboxamide

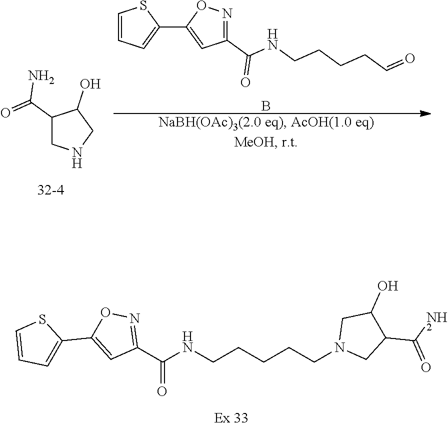

To a mixture of compound 32-4 (1.5 g, crude), Intermediate B (1.5 g, 5.38 mmol, 1.0 eq) and HOAc (323 mg, 5.38 mmol, 1.0 eq) in MeOH (30 mL) was added NaBH(OAc)₃ (2.28 g, 10.76 mmol, 2.0 eq) at 30° C. The mixture was stirred for 2 hours at 30° C. To the mixture was added sat.NaHCO₃ (10 mL). The mixture was concentrated under reduced pressure. The residue was purified by MPLC to give Example 33 (1.3 g, 100% purity) as a white solid, which was analyzed by LCMS and SFC. The product was purified by SFC to give four peaks, and then Prep-HPLC (base) to give peak 1 (119.34 mg, 100% purity), peak 2 (80.94 mg, 100% purity), peak 3 (55.48 mg, 100% purity) and peak 4 (154.01 mg, 100% purity.

Peak1: ¹H NMR (400 MHz, CD₃OD) δ ppm 7.69 (m, 2H), 7.23 (dd, J=3.8, 5.0 Hz, 1H), 6.92 (s, 1H), 4.50 (dt, J=3.8, 6.1 Hz, 1H), 3.41 (t, J=7.0 Hz, 2H), 3.10 (dd, 10.5 Hz, 1H), 3.05-2.89 (m, 3H), 2.63-2.50 (m, 3H), 1.75-1.53 (m, 4H), 1.52-1.37 (m, 2H). LC-MS: [M+H]⁺=393.3.

Peak2: ¹H NMR (400 MHz, CD₃OD) δ ppm 7.69 (m, 2H), 7.23 (dd, 5.0 Hz, 1H), 6.93 (s, 1H), 4.50 (dt, J=4.0, 5.8 Hz, 1H), 3.42 (t, J=7.1 Hz, 2H), 3.15-3.08 (m, 1H), 3.06-2.92 (m, 3H), 2.65-2.52 (m, 3H), 1.75-1.54 (m, 4H), 1.51-1.37 (m, 2H). LC-MS: [M+H]⁺=393.3.

Peak3: ¹H NMR (400 MHz, CD₃OD) δ ppm 7.69 (m, 2H), 7.23 (dd, J=3.8, 5.0 Hz, 1H), 6.93 (s, 1H), 4.50-4.41 (m, 1H), 3.41 (t, J=7.1 Hz, 2H), 3.10 (t, J=9.0 Hz, 1H), 2.85 (dt, J=4.6, 8.1 Hz, 1H), 2.74 (d, J=5.6 Hz, 2H), 2.65-2.41 (m, 3H), 1.75-1.53 (m, 4H), 1.51-1.36 (m, 2H). LC-MS: [M+H]⁺=393.3.

Peak4: ¹H NMR (400 MHz, CD₃OD) δ ppm 7.69 (m, 2H), 7.23 (dd, J=3.7, 5.1 Hz, 1H), 6.93 (s, 1H), 4.51-4.40 (m, 1H), 3.41 (t, J=7.1 Hz, 2H), 3.09 (t, J=8.8 Hz, 1H), 2.84 (dt, J=4.6, 8.1 Hz, 1H), 2.78-2.69 (m, 2H), 2.64-2.38 (m, 3H), 1.76-1.52 (m, 4H), 1.51-1.36 (m, 2H). LC-MS: [M+H]+=393.3.

### Example 34

### N-(5-((3S,4R)-3-cyano-4-hydroxypyrrolidin-1-yl)pentyl)-5-(thiophen-2-yl)isoxazole-3-carboxamide (Example 33-cis)

### N-(5-((3S,4S)-3-cyano-4-hydroxypyrrolidin-1-yl)pentyl)-5-(thiophen-2-yl)isoxazole-3-carboxamide (Example 33-trans)

### Step 1: 4-hydroxypyrrolidine-3-carbonitrile

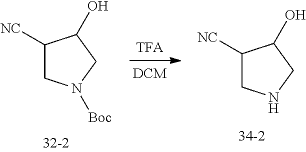 [SMILES: CC(C)(C)OC(=O)N1CC(O)C(C#N)C1 ; N#CC1CNCC1O]

To a solution of compound 32-2 (1.5 g, 7.07 mmol) in DCM (9 mL) was added TFA (3 mL) and stirred at 25° C. for 1 hours. TLC (PE:EA=2:1, Rf=0.1) indicated compound 32-2 was consumed. The mixture was concentrated in vacuo to give the title compound (2.1 g, crude) which was used in next step directly.

### Step 2: N-(5-(3-cyano-4-hydroxypyrrolidin-1-yl)pentyl)-5-(thiophen-2-yl)isoxazole-3-carboxamide

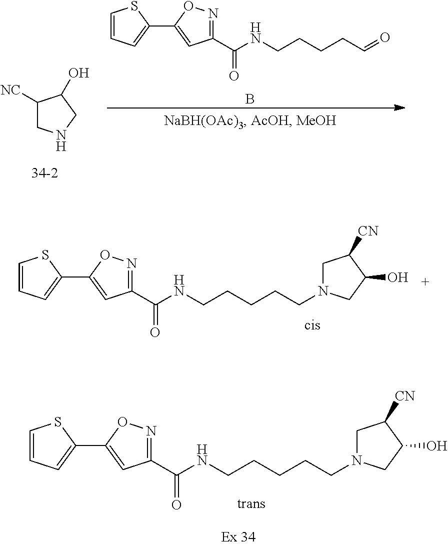

To a solution of Intermediate B (992.87 mg, 3.57 mmol) in MeOH (15 mL) was added compound 34-2 (1.0 g, 8.92 mmol), AcOH (535.56 mg, 8.92 mmol), NaBH(OAc)₃ (3.78 g, 17.84 mmol) and stirred at 25° C. for 16 hours. LCMS showed Intermediate B was consumed, the mixture was filtered and concentrated in vacuo to give a residue. The residue was purified by Prep-HPLC (basic condition) to give the title compounds (cis) (94.06 mg, 7.00% yield) and (trans) (193.83 mg, 14.28% yield) as white powder.

Example 34-cis: ¹H NMR (400 MHz, CD₃OD) δ ppm 7.72-7.61 (m, 2H), 7.20 (m, 1H), 6.90 (s, 1H), 4.47-4.38 (m, 1H), 3.43-3.34 (m, 2H), 3.30-3.25 (m, 1H), 3.11-2.97 (m, 2H), 2.78 (m, 1H), 2.57-2.41 (m, 3H), 1.65 (m, 2H), 1.55 (m, 2H), 1.42 (m, 2H). LC-MS: [M+H]⁺=375.3.

Example 34-trans: ¹H NMR (400 MHz, CD₃OD) δ ppm 7.58 (m, 2H), 7.13 (s, 1H), 6.82 (s, 1H), 4.40 (s, 1H), 3.31-3.27 (m, 1H), 3.23 (s, 1H), 3.00-2.90 (m, 1H), 2.90-2.77 (m, 2H), 2.65 (m, 1H), 2.50-2.30 (m, 3H), 1.58 (m, 2H), 1.46 (m, 2H), 1.34 (m, 2H). LC-MS: [M+H]⁺=375.3.

### Example 35

### N-(5-((3R,4S)-3-carbamoyl-4-hydroxypiperidin-1-yl)pentyl)-5-(thiophen-2-yl)isoxazole-3-carboxamide (cis)

### N-(5-((3R,4R)-3-carbamoyl-4-hydroxypiperidin-1-yl)pentyl)-5-(thiophen-2-yl)isoxazole-3-carboxamide (trans)

### Step 1: methyl 1-benzyl-4-oxopiperidine-3-carboxylate

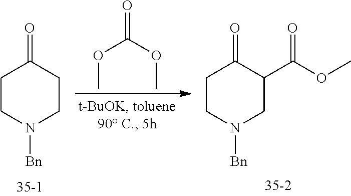

To a mixture of dimethyl carbonate (4.76 g, 52.84 mmol, 2.0 eq) and t-BuOK (6.67 g, 29.44 mmol, 2.25 eq) in toluene (60 mL) was added a solution of compound 35-1 (5 g, 26.42 mmol, 1.0 eq) dropwise at 90° C. The mixture was stirred for 2 hours at 90° C. To the reaction mixture was added AcOH (2.35 eq) and water (100 mL). The organic layer was separated and washed with brine (50 mL). The organic layer was dried over Na₂SO₄ and concentrated under reduced pressure to give the title compound (7 g, crude) as a yellow oil. LC-MS: [M+H]⁺=248.2.

### Step 2: methyl 1-benzyl-4-hydroxypiperidine-3-carboxylate

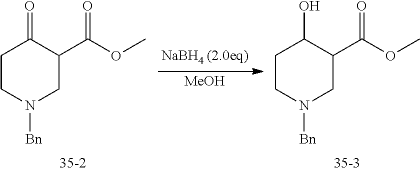 [SMILES: "MeOH" ; COC(=O)C1CN(Cc2ccccc2)CCC1=O ; COC(=O)C1CN(Cc2ccccc2)CCC1O]

To a mixture of compound 35-2 (2.0 g, 8.09 mmol, 1.0 eq) in MeOH (40 mL) was added NaBH₄ (611.95 mg, 16.18 mmol, 2.0 eq) at 0° C. The mixture was stirred for 2 hours at 0° C. The mixture was concentrated under reduced pressure to give a residue. To the residue was added EA (50 mL) and water (50 mL). The EA layer was washed with brine (50 ml), dried over Na₂SO₄ and concentrated under reduced pressure to give compound 35-3 (1.8 g, crude) as a yellow oil. LC-MS: [M+H]⁺=250.3.

### Step 3: 1-benzyl-4-hydroxypiperidine-3-carboxamide

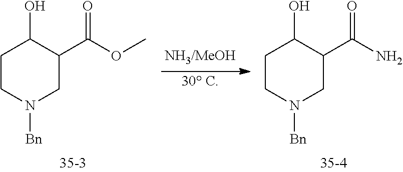 [SMILES: COC(=O)C1CN(Cc2ccccc2)CCC1O ; "30° C." ; NC(=O)C1CN(Cc2ccccc2)CCC1O]

A mixture of compound 35-3 in NH₃/MeOH (7M, 20 mL) was stirred for 16 h at 10 and for 120 h at 30° C. LCMS(C-05663-131-P1B4) showed little compound 35-3 was remained and desired mass was detected. The mixture was concentrated under reduced pressure to give the title compound (1 g, crude) as a yellow oil. LC-MS: [M+H]⁺=235.3.

### Step 4: 4-hydroxypiperidine-3-carboxamide

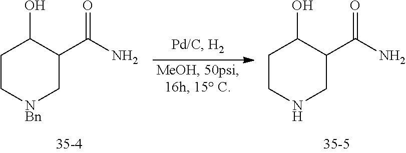 [SMILES: NC(=O)C1CN(Cc2ccccc2)CCC1O ; "MeOH, 50psi,16h, 15° C." ; NC(=O)C1CNCCC1O]

To a mixture of compound 35-4 (1.0 g, 4.27 mmol) in MeOH (20 mL) was added Pd/C (wet, 10%, 200 mg) at 15° C. The mixture was stirred for 16 h at 15° C. under H2 at 50 psi. The mixture was filtered and the filtrate was concentrated under reduced pressure to afford the title compound (600 mg, crude) as a yellow oil. LC-MS: [M+H]⁺=145.1.

### Step 5: N-(5-(3-carbamoyl-4-hydroxypiperidin-1-yl)pentyl)-5-(thiophen-2-yl)isoxazole-3-carboxamide

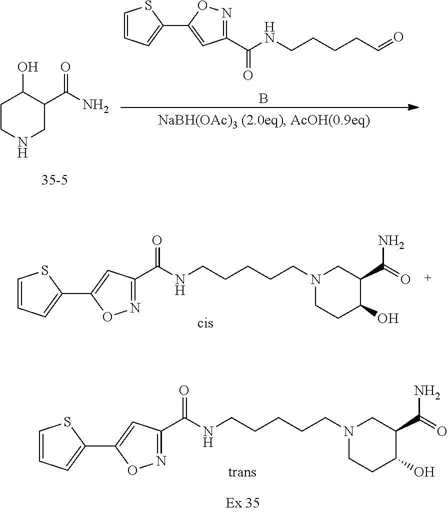

To a mixture of compound 35-5 (300 mg, 2.08 mmol, 1.0 eq) and Intermediate B in MeOH (10 mL) were added HOAc (112.46 mg, 1.87 mmol, 0.9 eq) and NaHB(OAc)₃ (882.03 mg, 4.16 mmol, 2.0 eq) at 10-15° C. The mixture was stirred for 16 h at 10-15° C. To the mixture was added sat.NaHCO₃ (2 mL). The mixture was concentrated under reduced pressure to give a residue. The residue was purified by Prep-HPLC (base) (Phenomenex Gemini 150*25 mm*10 um, gradient: 22-52% B (A=water (0.05% ammonia hydroxide v/v), B═CH₃CN), flow rate: 30 mL/min) to give the title compounds (trans) (102.45 mg,) and (cis) (122.73 mg) as white powder.

Example 35-trans: ¹H NMR (400 MHz, CD₃OD) δ ppm 7.69 (m, 2H), 7.23 (dd, J=3.7, 4.9 Hz, 1H), 6.92 (s, 1H), 3.74 (m, 1H), 3.41 (t, J=7.1 Hz, 2H), 3.08-2.91 (m, 2H), 2.48-2.36 (m, 3H), 2.21-2.06 (m, 2H), 2.01-1.89 (m, 1H), 1.74-1.53 (m, 5H), 1.49-1.35 (m, 2H). LC-MS: [M+H]⁺=407.3.

Example 35-cis: ¹H NMR (400 MHz, CD₃OD) δ ppm 7.69 (m, 2H), 7.23 (dd, 5.0 Hz, 1H), 6.92 (s, 1H), 4.15 (br s, 1H), 3.42 (t, J=7.1 Hz, 2H), 2.82-2.49 (m, 5H), 2.48-2.36 (m, 2H), 1.86-1.76 (m, 2H), 1.74-1.55 (m, 4H), 1.50-1.37 (m, 2H). LC-MS: [M+H]⁺=407.3.

### Example 36

### N-(5-(3-((4-hydroxytetrahydrofuran-3-yl)carbamoyl)azetidin-1-yl)pentyl)-5-(thiophen-2-yl)isoxazole-3-carboxamide

### Step 1: methyl 1-(5-(5-(thiophen-2-yl)isoxazole-3-carboxamido)pentyl)azetidine-3-carboxylate

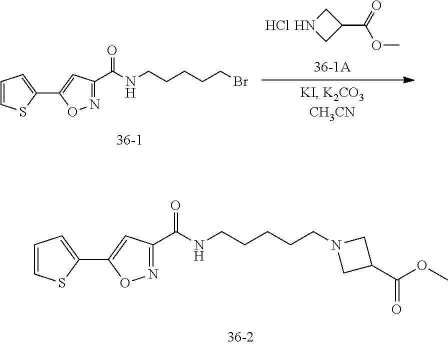

To a suspension of compound 36-1 (968 mg, 2.82 mmol, 1 eq) in CH₃CN (15 mL) was added K₂CO₃ (1.17 g, 8.45 mmol, 3 eq) and KI (467 mg, 2.82 mmol, 1 eq) at 0° C. After addition, compound 36-1A (512 mg, 3.38 mmol, 1.2 eq) was added and the reaction mixture was stirred at 9-16° C. for 18 hours. LCMS showed the reaction was completed. The mixture was filtered. The filtrate was concentrated under reduced pressure and the residue was purified by column on silica gel (DCM:MeOH=50:1 to 10:1) to afford the compound (1.05 g, crude) as a light yellow oil. LC-MS: [M+H]⁺=378.2.

### Step 2: 1-(5-(5-(thiophen-2-yl)isoxazole-3-carboxamido)pentyl)azetidine-3-carboxylic acid

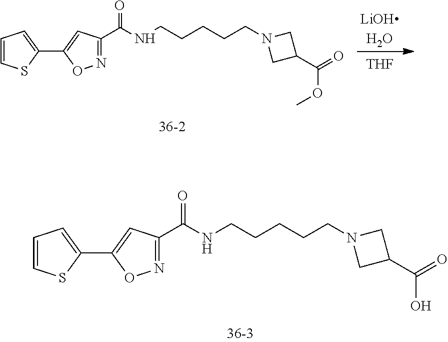 [SMILES: COC(=O)C1CN(CCCCCNC(=O)c2cc(-c3cccs3)on2)C1 ; "LiOH·H2O" ; O=C(NCCCCCN1CC(C(=O)O)C1)c1cc(-c2cccs2)on1 ; "THF"]

To a stirred solution of compound 36-2 (1.05 g, 2.78 mmol, 1.0 eq) in H₂O/THF (8 mL/16 mL) was added LiOH.H₂O (350 mg, 8.35 mmol, 3.0 eq) at 0° C. Then the mixture was stirred at 12-19° C. for 18 hours. LCMS showed the reaction was completed. Acidify the reaction mixture by adding, with shaking, 1N HCl to adjust pH to 5-6, and then removed the THF under reduced pressure and the residual aqueous was lyophilized to afford the title compound (2.78 mmol) as a yellow gum. LC-MS: [M+H]⁺=364.1.

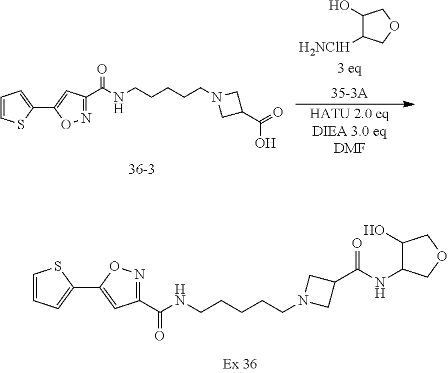

To a stirred solution of compound 36-3 (110 mg, 0.302 mmol, 1.0 eq) in DMF (1 mL) was added DIEA (196 mg, 1.51 mmol, 5 eq) and compound 36-3A (127 mg, 0.908 mmol, 3 eq) at 11-14° C. Then the mixture was stirred at 11-14° C. for 3 min, HATU (230 mg, 0.605 mmol, 2 eq) was added. After addition, the reaction was stirred at 11-14° C. for 18 hours. LCMS showed the reaction was completed. The mixture was diluted with DMF (1.5 mL) and purified by basic prep-HPLC (Column Xtimate C18 150*25 mm*5 um, gradient: 22-52% B (A=water (0.05% ammonia hydroxide v/v) B═CH₃CN), flow rate: 25 ml/min) to afford the title compound (42 mg, 96.06% purity) as a white solid. ¹H NMR (400 MHz, CD₃OD) δ ppm 7.69-7.66 (m, 2H), 7.21 (dd, J=4.0, 4.8 Hz, 1H), 6.88 (s, 1H), 4.18-4.09 (m, 2H), 4.07-4.01 (m, 1H), 3.96-3.91 (m, 1H), 3.67-3.60 (m, 2H), 3.55-3.49 (m, 2H), 3.38 (t, J=6.8 Hz, 2H), 3.29-3.25 (m, 3H), 2.55-2.47 (m, 2H), 1.69-1.57 (m, 2H) 1.45-1.35 (m, 4H). LC-MS: [M+H]⁺=449.2.

### Example 37

### N-(5-(4-(3-amino-2-hydroxy-3-oxopropyl)piperazin-1-yl)pentyl)-5-(thiophen-2-yl)isoxazole-3-carboxamide

### Step 1: tert-butyl 4-(2-hydroxy-3-methoxy-3-oxopropyl)piperazine-1-carboxylate

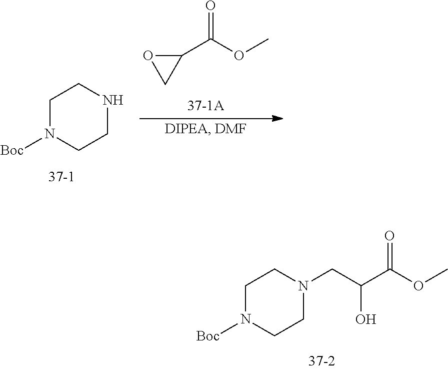

To a solution of compound 37-1 (1.0 g, 5.37 mmol) and compound 37-1A (657.75 mg, 6.44 mmol, 1.2 eq) in DMF (10 ml) was added DIPEA (2.08 g, 16.11 mmol, 3.0 eq) at 20° C. Then, the reaction was heated to 80° C. for 16 hours. TLC (DCM/MeOH=10/1) showed all the starting material was consumed, and a main new spot was found. The reaction was diluted with EA (100 ml), washed with water (30 ml). The organic layer was concentrated in vacuo to give compound 37-2 (1.4 g, crude) as yellow oil. It was used directly for next step. ¹H NMR (400 MHz, CDCl₃) δ ppm 4.23 (dd, J=4.0, 7.6 Hz, 1H), 3.72 (s, 3H), 3.40-3.31 (m, 4H), 2.75-2.58 (m, 2H), 2.56-2.46 (m, 2H), 2.42-2.33 (m, 2H), 1.45-1.34 (s, 9H)

### Step 2: tert-butyl 4-(3-amino-2-hydroxy-3-oxopropyl)piperazine-1-carboxylate

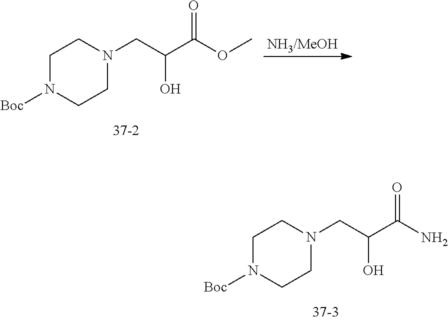 [SMILES: COC(=O)C(O)CN1CCN(C(=O)OC(C)(C)C)CC1 ; "NH3/MeOH" ; CC(C)(C)OC(=O)N1CCN(CC(O)C(N)=O)CC1]

A solution of compound 37-2 (200 mg, 693.63 umol) in NH₃.MeOH (10 ml, 4N) was stirred at 20° C. for 16 hours. LCMS showed the desired product was found as main peak. The reaction was concentrated in vacuo to give compound 37-3 (200 mg, crude) as white solid. The crude product was used directly for next step without purification. LC-MS: [M+H]⁺=274.3.

### Step 3: 2-hydroxy-3-(piperazin-1-yl)propanamide

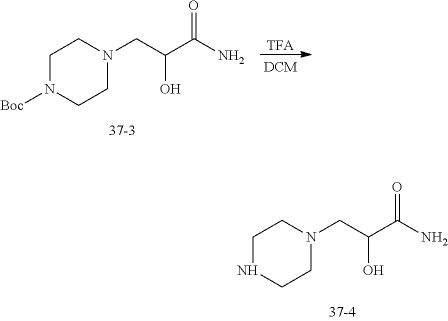 [SMILES: CC(C)(C)OC(=O)N1CCN(CC(O)C(N)=O)CC1 ; NC(=O)C(O)CN1CCNCC1 ; "TFA"]

To a solution of compound 37-3 (200 mg, 731.72 umol) in DCM (4 ml) was added TFA (2 ml) at 20° C., and it was stirred at 20° C. for 3 hours. The reaction mixture was concentrated in vacuum to give compound 37-4 (200 mg, crude) as yellow oil. The crude product was used directly for next step without purification.

### Step 4: N-(5-(4-(3-amino-2-hydroxy-3-oxopropyl)piperazin-1-yl)pentyl)-5-(thiophen-2-yl)isoxazole-3-carboxamide

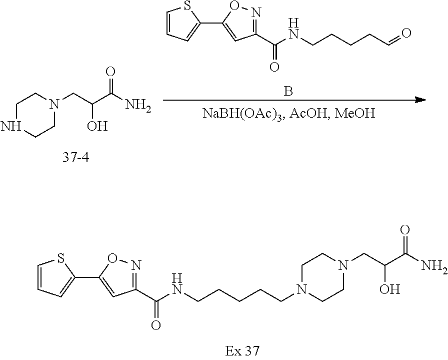

To a solution of compound 37-4 (200 mg, 742.88 umol), Intermediate B (206.76 mg, 742.88 umol) and AcOH (44.61 mg, 742.88 mg) in MeOH (10 ml) was added NaBH(OAc)₃ (472.34 mg, 2.23 mmol) at 20° C., and it was stirred at 20° C. for 16 hours. LCMS showed all the starting material was consumed, and desired product was found. The reaction was diluted with EA (20 ml) and water (10 ml). The organic layer was separated and concentrated in vacuo. The residue was purified by prep-HPLC (base) to give Example 37 (16.3 mg, 5.06% yield) as white solid. ¹H NMR (400 MHz, CD₃OD) δ ppm 7.71-7.68 (m, 2H), 7.23 (dd, J=3.8, 4.9 Hz, 1H), 6.92 (s, 1H), 4.17 (dd, J=3.5, 8.3 Hz, 1H), 3.41 (t, J=7.1 Hz, 2H), 2.78-2.47 (m, 10H), 2.45-2.35 (m, 2H), 1.75-1.54 (m, 4H), 1.48-1.36 (m, 2H). LC-MS: [M+H]⁺=436.4.

### (R)—N-(5-(4-(3-amino-2-hydroxy-3-oxopropyl)piperazin-1-yl)pentyl)-5-(thiophen-2-yl)isoxazole-3-carboxamide

### (S)—N-(5-(4-(3-amino-2-hydroxy-3-oxopropyl)piperazin-1-yl)pentyl)-5-(thiophen-2-yl)isoxazole-3-carboxamide

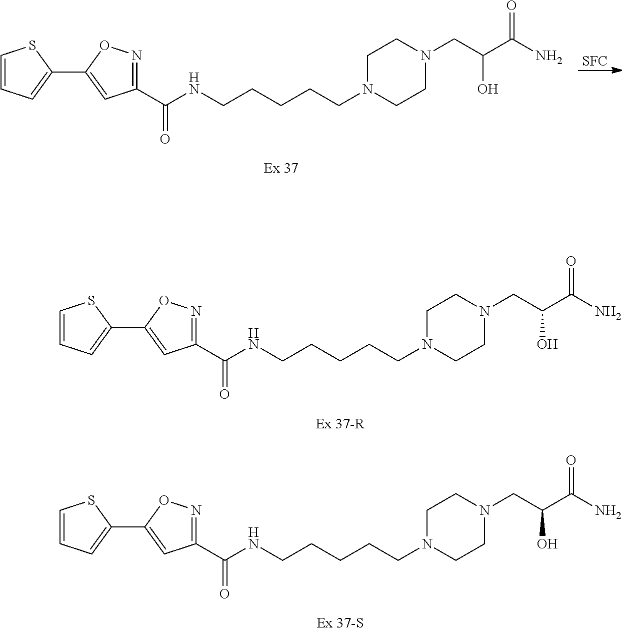

Example 37-R: ¹H NMR (400 MHz, CD₃OD) δ ppm 7.69 (ddd, J=1.0, 4.4, 9.2 Hz, 2H), 7.22 (dd, J=3.8, 5.0 Hz, 1H), 6.92 (s, 1H), 4.17 (dd, J=3.6, 8.3 Hz, 1H), 3.41 (t, J=7.1 Hz, 2H), 2.77-2.46 (m, 10H), 2.44-2.36 (m, 2H), 1.73-1.54 (m, 4H), 1.49-1.37 (m, 2H). C-05707-094-P2A. LC-MS: [M+H]⁺=436.0.

Example 37-S: ¹H NMR (400 MHz, CD₃OD) δ ppm 7.57 (ddd, J=1.1, 4.3, 9.2 Hz, 2H), 7.11 (dd, J=3.7, 5.0 Hz, 1H), 6.80 (s, 1H), 4.05 (dd, J=3.6, 8.3 Hz, 1H), 3.29 (t, J=7.0 Hz, 2H), 2.66-2.33 (m, 10H), 2.32-2.24 (m, 2H), 1.61-1.42 (m, 4H), 1.36-1.25 (m, 2H). LC-MS: [M+H]⁺=436.0.

### Example 38

### Atoh1 induction assay in mouse cerebellar neural precursor cells (NPCs)

Atoh1 induction assay was conducted with in vitro cultured cerebellar neural precursor cells isolated from neonatal transgenic Atoh1-GFP mice. Atoh1 expression is mainly regulated by the enhancer, and the nuclear GFP was driven by the cloned enhancer sequence at 3′ of Atoh1 which had high conservation among mammalians So Atoh1 induction could be reflected by GFP activation in cerebellar neural precursor cells (Helms et al., Development 2000; 127: 1185-1196; Lumpkin et al., Gene Expression Patterns 2003; 3: 389-395). Postnatal 3 days pups were dissected for cerebellum tissue isolation. The cerebellum tissue was cut into small pieces, and dissociated with 0.05% Trypsin for about 10 minutes at 37° C., and then filtered with a 70 uM cell strainer. The cells were cultured as neuropsheres for the first 2 days in ultra-low attachment dish/well-plate with DMEM/F12+1% N₂ &2% B27 with 1% P/S, 20 ng/ml rhFGF2 and 20 ng/ml rhEGF (R&D Systems). Then the spheres were plated to the matrigel (1:30 diluted in DMEM/F12)-coated tissue culture dish for monolayer culture. After 4.5-5.5 days culture in vitro (DIV), cells were dissociated with 0.05% trypsin into single cells, and frozen after cell number calculation.

The cerebellar neural precursor cells (NPCs) were re-thawed from stock and cultured for another 2 days before used for Atoh1 induction assay. On the first day of assay, NPCs were seeded into matrigel-coated 384 well plates (Black view-plate, PE) at 2500 cells/well. After over-night culture, the NPCs were treated with representative compounds of the present disclosure with 1:2 serial dilutions for 10 doses, from 50 μM to 200 nM, with DMSO as negative control. After 72 hours treatment without medium change, the cells were fixed with 4% formalin for staining Assay plates were stained with GFP antibody (Abcam, #13970, 1:1000) to amplify endogenous GFP signal and then read by Cellomics. The GFP average intensity in cell nuclie which is defined by DAPI staining for the tested compounds were calculated and compared to DMSO control, and the difference is expressed in a fold difference format according to the equation of (the GFP average intensity of the tested compound/(the DMSO control). The maximum fold difference of each tested compound over the DMSO control is described in below Table 1 (see the column with the title “fold difference”). Note the value of the DMSO control is 1 in the equation, and any fold difference more than 5 is considered as a significant difference. As shown in Table 1, all of the tested compounds of the present disclosure have demonstrated significant fold difference in terms of GFP average intensity over the DMSO control. Therefore, all of the tested compounds were active for the activation of Atoh1 and significantly increase the Atoh1 expression. Table 1 Atoh1 activation by treatment of Compounds of the present disclosure (Atoh1-GFP reporter assay in cerebellar NPCs)

| Example | Fold Difference* |
|---|---|
| DMSO | 1.0 |
| 3 | 11.2 |
| 34 cis | 11.2 |
| 2 | 11.5 |
| 28-cis | 11.9 |
| 1 | 12.7 |
| 34 trans | 13.1 |
| 25 | 13.1 |
| 27 | 13.5 |
| 36 | 13.6 |
| 8 | 14.6 |
| 31 | 14.7 |
| 20 | 14.7 |
| 4 | 18.3 |
| 29 | 15.4 |
| 30 | 16.1 |
| 26 | 16.3 |
| 28-trans | 16.8 |
| 22 | 16.8 |
| 5 | 17.2 |
| 7 | 17.2 |
| 24 | 18.3 |
| 15 | 18.9 |
| 10 | 19.1 |
| 6 | 20.1 |
| 18 | 20.4 |
| 17 | 20.4 |
| 21 | 20.8 |
| 12 | 22.4 |
| 19 | 16.3 |
| 32 peak 4 | 23.2 |
| 14 | 23.8 |
| 23 | 24.3 |
| 9 | 24.4 |
| 33 peak 3 | 27.3 |
| 35 trans | 24.8 |
| 32 peak 3 | 27.6 |
| 37 | 27.6 |
| 33 peak 2 | 35.6 |
| 37 R | 29.2 |
| 13 | 32.5 |
| 33 peak 1 | 34.8 |
| 37 S | 31.5 |
| 16 | 32.3 |
| 35 cis | 33.2 |
| 11 | 33.3 |
| 33 peak 4 | 33.7 |

*The ratio of Atoh1-GFP average intensity_FC to DMSO_Max

Ex Vivo Hair Cell Induction Assay Using 6-Day-Postnatal Mouse Cochlea Explants with Hair Cell Damage

P6, postnatal 6 days, Atoh1-GFP mice, the same mouse strain used for Atoh1 induction assay described before, were used in this assay. The otic capsule was exposed and the cochleae were micro-dissected. The basilar membrane was separated from the organ of Corti and in vitro cultured in serum free medium (culture medium: DMEM/F12+1% N2+2% B27+5 μg/ml ampicillin) at 37° C. under a standard gas atmosphere of humidified air/5% CO₂. Inner ear hair cells were damaged by 1 mM Neomycin treatment for 1.25 k After the neomycin treatment, explants were cultured in blank culture medium for 7 days before the treatment of selected compounds.

For compound administration, the cochlea explants were treated with 3 to 10 μM compound of the present disclosure, with DMSO as the negative control for 8 days with once compound/medium change. After 8 days treatment, the tested compound was removed. The explants were cultured in blank medium for additional 4 days. The cochlea explant cultures then were fixed with 4% w/v formalin and processed for Myo7a immunofluorescence (Myo7a is a specific marker for sensory hair cell) using the rabbit anti-Myo7a antibody (Protus Biosci #25-6790, 1:250 diluted in PBS containing 3% BSA). Rhodamine labeled Goat-anti-rabbit IgG (Molecular Prob. #R6394, 1:1000 diluted in PBS containing 3% BSA) was used as the secondary antibody to visualize the Myo7a positive cells. The images were collected and analyzed using the EVOS image system (Thermo-Fisher Scientific). It was found that treatment with tested compounds significantly increased the number of Atoh1-GFP and Myo7a positive cells. The hair cell identity of the ectopically formed cells was confirmed by staining the cells with multiple hair cell markers.

The efficacy of hair cells induction in this assay is represented by the responsive length percentage of Atoh1 and Myo7a double positive cells in the damaged whole explants after compound treatment. The responsive length percentage was calculated according to the equation of ((the explant length with Atoh1 and Myo7a double positive cells/the full length of cochlea explant)*100%). Note the value of DMSO control is 0% due to total damage of hair cells, and any responsive length percentage more than 20% is considered as significant hair cell induction. As shown in Table 2, representative compounds of the present disclosure have demonstrated significant hair cell induction.

TABLE 2
Atoh1-GFP/Myo7a cells induction in cochlea explants
by treatment of compounds of the present disclosure
(final concentration 10 μM), Mean ± SD.

| | | | Atoh1-GFP/Myo7a |
|---|---|---|---|
| | | Response ratio | cell numbers in |
| | | (Response length/ | 25%-50% length |
| Compound | Concentration | full length %) | from Apex) |
| DMSO | | 0 ± 0 | 0 ± 0 |
| 18 | 10 μM | 59.4 ± 5.3 | 16.1 ± 3.0 |
| 33 peak 3 | 10 μM | 54.1 ± 5.3 | 19.8 ± 1.0 |
| 35 trans | 10 μM | 63.9 ± 5.8 | 14.2 ± 1.8 |
| 32 peak 3 | 10 μM | 56.3 ± 2.8 | 15.4 ± 0.2 |
| 37 | 10 μM | 56.9 ± 8.8 | 16.3 ± 4.0 |
| 33 peak 2 | 10 μM | 66.6 ± 4.0 | 26.9 ± 0.4 |
| 37-R | 10 μM | 42.2 ± 7.7 | 13.6 ± 4.0 |
| 33 peak 1 | 10 μM | 62.1 ± 11.8 | 19.4 ± 2.7 |
| 37-S | 10 μM | 50.1 ± 16.8 | 11.1 ± 3.0 |
| 33 peak 4 | 10 μM | 48.3 ± 1.0 | 13.6 ± 1.1 |

Note:
the responsive length % is a mean ± SD.
SD: standard deviation

1. A compound of Formula (I) 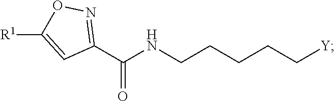
or a pharmaceutically acceptable salt thereof,
wherein:
R¹ is selected from: 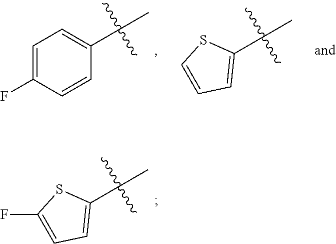 [SMILES: *c1ccc(F)cc1 ; *c1cccs1 ; *c1ccc(F)s1]
Y is selected from 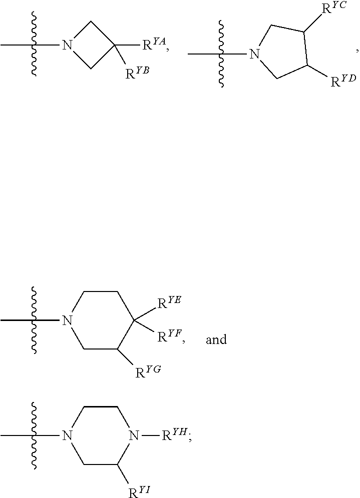
R^{YA} and R^{YB} are independently selected from H, —S(═O)₂NH(R²), —(C═O)NH(R²), —NH(C═O)OCH₂(C═O)NH(R²), —CH₂OH, —CH₃, and —OH;
R^{YC} and R^{YD} are independently selected from H, —CN, —OH, —(C═O)NH₂, and —S(═O)₂NH(R²);
R^{YE} and R^{YF} are independently selected from H, —CN, —(C═O)NH(R²), —OH, and —S(═O)₂NH(R²);
R^{YG} is selected from H, —CN, —OH, —F, —(C═O)NH(R²), and —S(═O)₂NH(R²);
R^{YH} is selected from —CH₃, —(X¹)—(C═O)NH(R²), and —(X¹)—S(═O)₂NH(R²);
R^{YI} is selected from —H and —(C═O)NH(R²);
X¹ is C₀₋₂ alkylene, optionally substituted with —OH;
R² is independently selected from H, —CH₃, —CH(CH₃)CN, —CH₂CH₂CN, 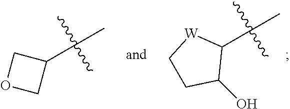
and
W is O or CH₂;
wherein when R¹ is: 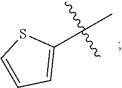 [SMILES: *c1cccs1]
Y is not: 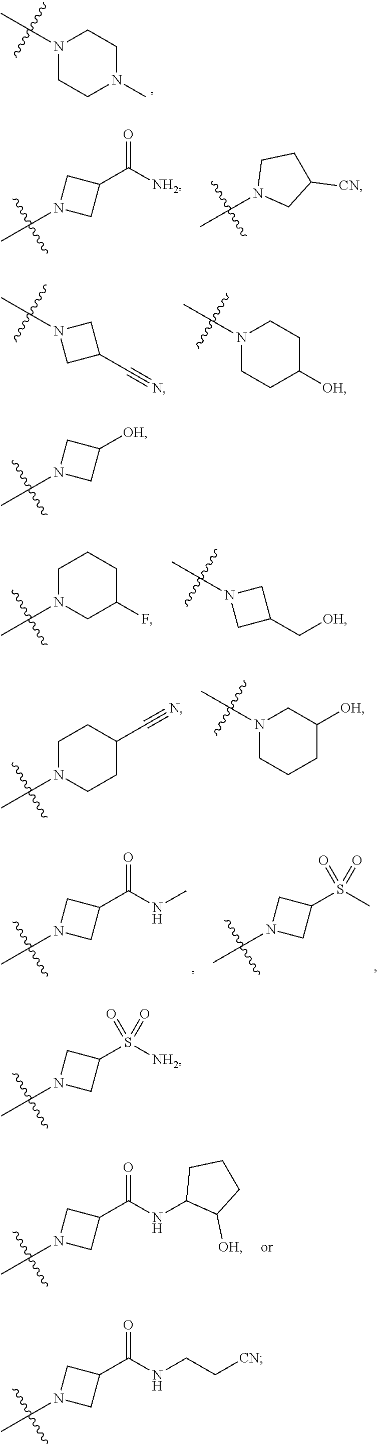
when R¹ is: 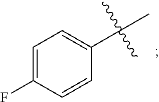 [SMILES: *c1ccc(F)cc1]
Y is not: 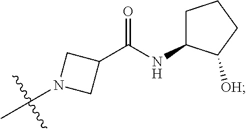 [SMILES: *N1CC(C(=O)N[C@H]2CCC[C@@H]2O)C1]
and
when R¹ is: 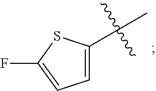 [SMILES: *c1ccc(F)s1]
Y is not:

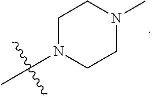 [SMILES: *N1CCN(C)CC1]

2. A compound of Formula (I) 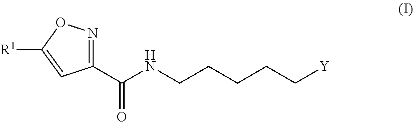
or a pharmaceutically acceptable salt thereof,
wherein:
R¹ is selected from: 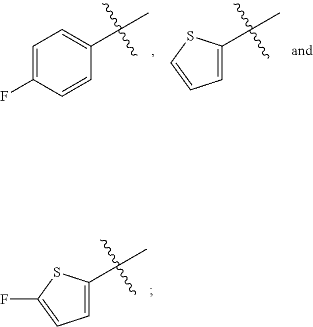 [SMILES: *c1ccc(F)cc1 ; *c1cccs1 ; *c1ccc(F)s1]
Y is selected from 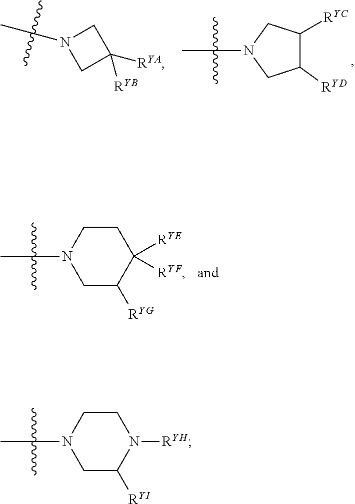
R^{YA} and R^{YB} is independently selected from H, —S(═O)₂NH(R²), —(C═O)NH(R²), —NH(C═O)OCH₂(C═O)NH(R²), —CH₂OH, —CH₃, and —OH;
R^{YC} and R^{YD} is independently selected from H, —CN, —OH, —(C═O)NH₂, and —S(═O)₂NH(R²);
R^{YE} and R^{YF} is independently selected from H, —CN, —(C═O)NH(R²), —OH, and —S(═O)₂NH(R²);
R^{YG} is selected from H, —CN, —OH, —F, —(C═O)NH(R²), and —S(═O)₂NH(R²);
R^{YH} is selected from —CH₃, —(X¹)—(C═O)NH(R²), and —(X¹)—S(═O)₂NH(R²);
R^{YI} is selected from —H and —(C═O)NH(R²);
X¹ is C₀₋₂ alkylene, optionally substituted with —OH;
R² is independently selected from H, —CH₃, —CH(CH₃)CN, —CH₂CH₂CN, 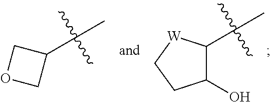
and
W is O or CH₂;
wherein the compound is not:

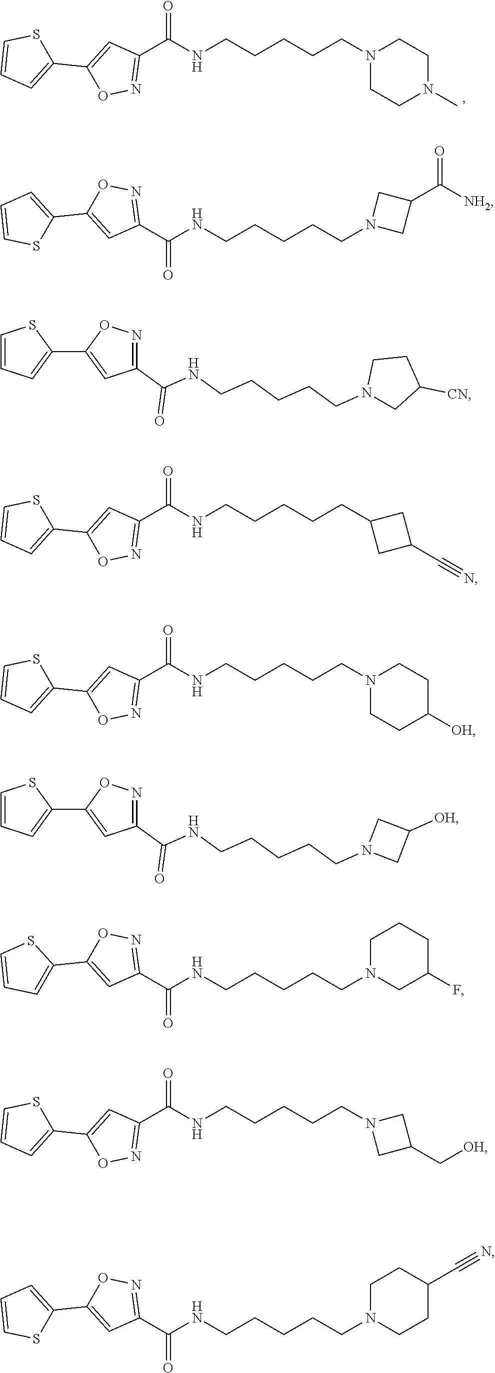

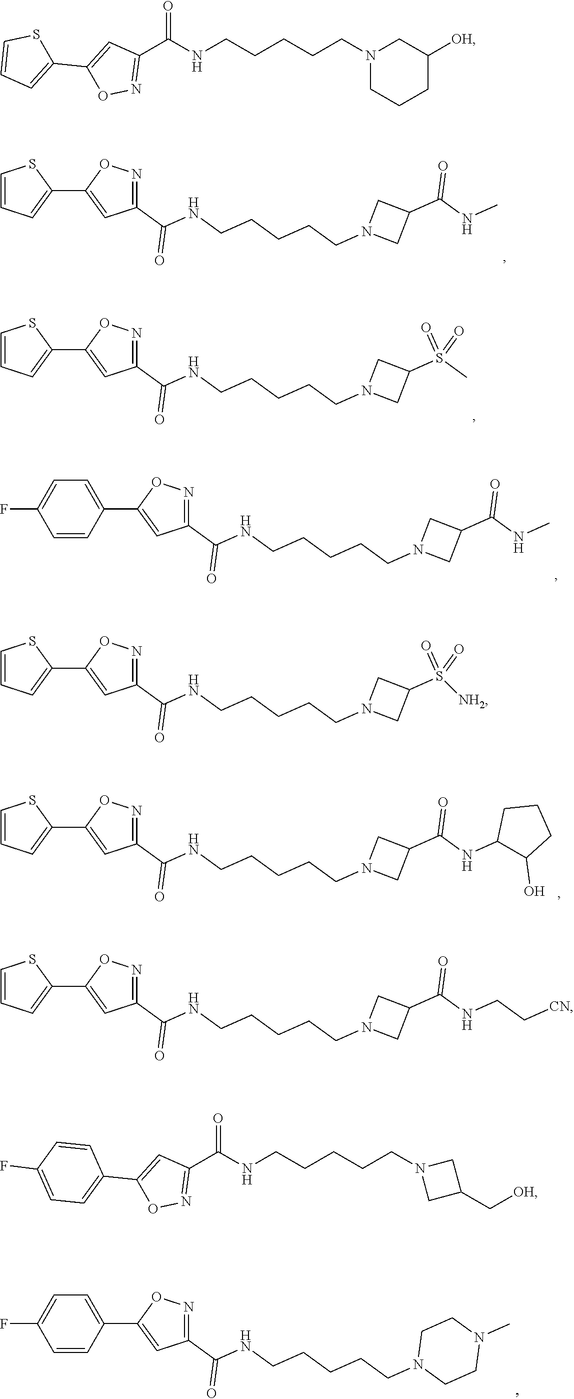

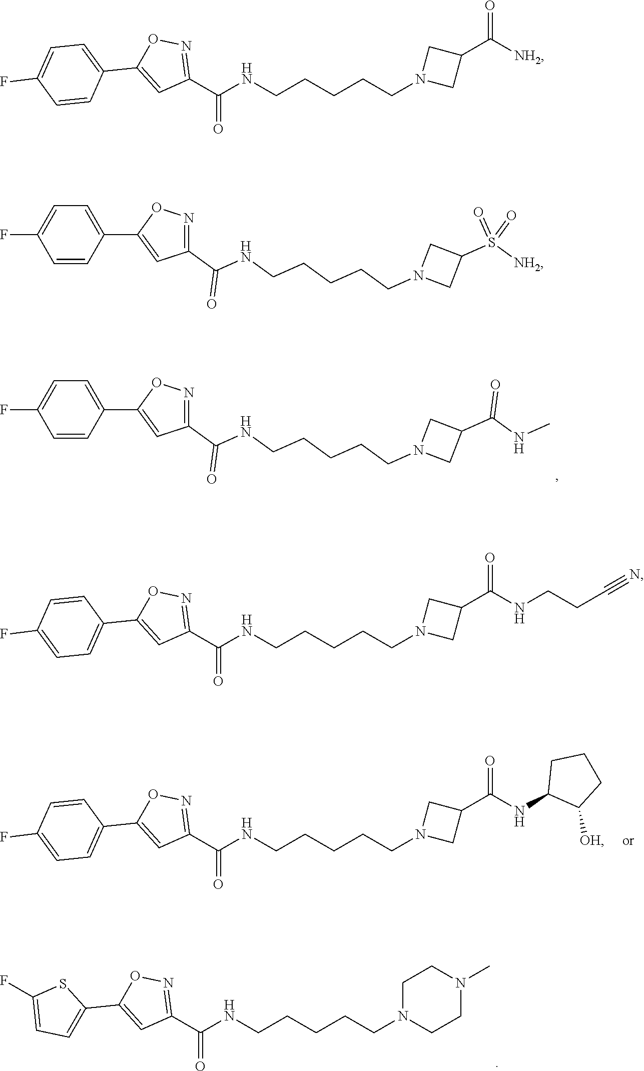 [SMILES: NC(=O)C1CN(CCCCCNC(=O)c2cc(-c3ccc(F)cc3)on2)C1 ; NS(=O)(=O)C1CN(CCCCCNC(=O)c2cc(-c3ccc(F)cc3)on2)C1 ; CNC(=O)C1CN(CCCCCNC(=O)c2cc(-c3ccc(F)cc3)on2)C1 ; N#CCCNC(=O)C1CN(CCCCCNC(=O)c2cc(-c3ccc(F)cc3)on2)C1 ; O=C(NCCCCCN1CC(C(=O)N[C@H]2CCC[C@@H]2O)C1)c1cc(-c2ccc(F)cc2)on1 ; CN1CCN(CCCCCNC(=O)c2cc(-c3ccc(F)s3)on2)CC1]

3. The compound of claim 1 or a pharmaceutically acceptable salt thereof, wherein:
R^{YG} is selected from H, —CN, —OH, —F, —(C═O)NH₂, and —S(═O)₂NH₂;
R^{YH} is selected from —CH₃, —(X¹)—(C═O)NH₂, and —(X¹)—S(═O)₂NH₂;
R^{YI} is selected from —H and —(C═O)NH₂;
X¹ is C₁₋₂ alkylene, optionally substituted with OH.

4. The compound of claim 1 or a pharmaceutically acceptable salt thereof, wherein:
R¹ is:

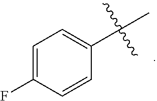 [SMILES: *c1ccc(F)cc1]

5. The compound of claim 1 or a pharmaceutically acceptable salt thereof, wherein:
R¹ is:

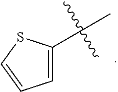 [SMILES: *c1cccs1]

6. The compound of claim 1 or a pharmaceutically acceptable salt thereof, wherein:
R¹ is:

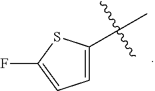 [SMILES: *c1ccc(F)s1]

7. The compound of claim 1 or a pharmaceutically acceptable salt thereof, wherein:
Y is

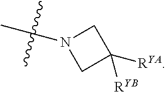

8. The compound of claim 1 or a pharmaceutically acceptable salt thereof, wherein:
Y is

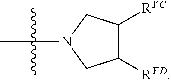

9. The compound of claim 1 or a pharmaceutically acceptable salt thereof, wherein:
Y is

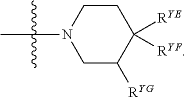

10. The compound of claim 1 or a pharmaceutically acceptable salt thereof, wherein:
Y is

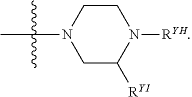

11. The compound or a pharmaceutically acceptable salt thereof according to claim 1 selected from the following: N-(5-((3S,4S)-4-carbamoyl-3-cyanopiperidin-1-yl)pentyl)-5-(thiophen-2-yl)isoxazole-3-carboxamide; N-(5-((3S,4R)-4-cyano-3-hydroxypiperidin-1-yl)pentyl)-5-(4-fluorophenyl)isoxazole-3-carboxamide; N-(5-(3-(N-(oxetan-3-yl)sulfamoyl)azetidin-1-yl)pentyl)-5-(thiophen-2-yl)isoxazole-3-carboxamide; N-(5-(3-(N-(2-cyanoethyl)sulfamoyl)azetidin-1-yl)pentyl)-5-(thiophen-2-yl)isoxazole-3-carboxamide; 2-(methylamino)-2-oxoethyl (1-(5-(5-(thiophen-2-yl)isoxazole-3-carboxamido)pentyl)azetidin-3-yl)carbamate; N-(5-(3-sulfamoylpiperidin-1-yl)pentyl)-5-(thiophen-2-yl)isoxazole-3-carboxamide; N-(5-(3-sulfamoylpyrrolidin-1-yl)pentyl)-5-(thiophen-2-yl)isoxazole-3-carboxamide; N-(5-(3-carbamoylpyrrolidin-1-yl)pentyl)-5-(4-fluorophenyl)isoxazole-3-carboxamide; N-(5-(3-carbamoyl-3-(hydroxymethyl)azetidin-1-yl)pentyl)-5-(thiophen-2-yl)isoxazole-3-carboxamide; N-(5-(3-carbamoyl-3-(hydroxymethyl)azetidin-1-yl)pentyl)-5-(4-fluorophenyl)isoxazole-3-carboxamide; N-(5-(3-carbamoyl-3-methylazetidin-1-yl)pentyl)-5-(thiophen-2-yl)isoxazole-3-carboxamide; N-(5-(3-carbamoyl-3-methylazetidin-1-yl)pentyl)-5-(4-fluorophenyl)isoxazole-3-carboxamide; N-(5-(3-carbamoyl-4-hydroxypyrrolidin-1-yl)pentyl)-5-(thiophen-2-yl)isoxazole-3-carboxamide; N-(5-(4-sulfamoylpiperidin-1-yl)pentyl)-5-(thiophen-2-yl)isoxazole-3-carboxamide; 5-(4-fluorophenyl)-N-(5-(4-sulfamoylpiperidin-1-yl)pentyl)isoxazole-3-carboxamide; N-(5-((3R,4R)-4-cyano-3-hydroxypiperidin-1-yl)pentyl)-5-(thiophen-2-yl)isoxazole-3-carboxamide; N-(5-(4-(3-amino-3-oxopropyl)piperazin-1-yl)pentyl)-5-(thiophen-2-yl)isoxazole-3-carboxamide; N-(5-(4-(2-amino-2-oxoethyl)piperazin-1-yl)pentyl)-5-(thiophen-2-yl)isoxazole-3-carboxamide; N-(5-(4-(2-sulfamoylethyl)piperazin-1-yl)pentyl)-5-(thiophen-2-yl)isoxazole-3-carboxamide; N-(5-(4-carbamoylpiperidin-1-yl)pentyl)-5-(thiophen-2-yl)isoxazole-3-carboxamide; N-(5-(3-carbamoyl-3-hydroxyazetidin-1-yl)pentyl)-5-(thiophen-2-yl)isoxazole-3-carboxamide; 5-(5-fluorothiophen-2-yl)-N-(5-(3-(methylcarbamoyl)azetidin-1-yl)pentyl)isoxazole-3-carboxamide; N-(5-(3-carbamoylazetidin-1-yl)pentyl)-5-(5-fluorothiophen-2-yl)isoxazole-3-carboxamide; N-(5-(3-((1-cyanoethyl)carbamoyl)azetidin-1-yl)pentyl)-5-(4-fluorophenyl)isoxazole-3-carboxamide; N-(5-(3-carbamoyl-4-methylpiperazin-1-yl)pentyl)-5-(thiophen-2-yl)isoxazole-3-carboxamide; N-(5-(4-carbamoyl-4-hydroxypiperidin-1-yl)pentyl)-5-(thiophen-2-yl)isoxazole-3-carboxamide; 5-(4-fluorophenyl)-N-(5-(3-(((1S,2R)-2-hydroxycyclopentyl)carbamoyl)azetidin-1-yl)pentyl)isoxazole-3-carboxamide; N-(5-((3R,4S)-4-carbamoyl-3-fluoropiperidin-1-yl)pentyl)-5-(thiophen-2-yl)isoxazole-3-carboxamide, and N-(5-((3S,4S)-4-carbamoyl-3-fluoropiperidin-1-yl)pentyl)-5-(thiophen-2-yl)isoxazole-3-carboxamide; N-(5-((3R,4S)-3-carbamoyl-4-cyanopiperidin-1-yl)pentyl)-5-(thiophen-2-yl)isoxazole-3-carboxamide; N-(5-(4-carbamoyl-3-hydroxypiperidin-1-yl)pentyl)-5-(thiophen-2-yl)isoxazole-3-carboxamide; N-(5-(4-carbamoyl-3-hydroxypiperidin-1-yl)pentyl)-5-(4-fluorophenyl)isoxazole-3-carboxamide; N-(5-((3S,4S)-3-carbamoyl-4-hydroxypyrrolidin-1-yl)pentyl)-5-(4-fluorophenyl)isoxazole-3-carboxamide; N-(5-((3S,4R)-3-carbamoyl-4-hydroxypyrrolidin-1-yl)pentyl)-5-(4-fluorophenyl)isoxazole-3-carboxamide; N-(5-((3R,4S)-3-carbamoyl-4-hydroxypyrrolidin-1-yl)pentyl)-5-(4-fluorophenyl)isoxazole-3-carboxamide; N-(5-((3R,4R)-3-carbamoyl-4-hydroxypyrrolidin-1-yl)pentyl)-5-(4-fluorophenyl)isoxazole-3-carboxamide; N-(5-((3S,4S)-3-carbamoyl-4-hydroxypyrrolidin-1-yl)pentyl)-5-(thiophen-2-yl)isoxazole-3-carboxamide; N-(5-((3R,4S)-3-carbamoyl-4-hydroxypyrrolidin-1-yl)pentyl)-5-(thiophen-2-yl)isoxazole-3-carboxamide; N-(5-((3R,4R)-3-carbamoyl-4-hydroxypyrrolidin-1-yl)pentyl)-5-(thiophen-2-yl)isoxazole-3-carboxamide; N-(5-((3S,4R)-3-carbamoyl-4-hydroxypyrrolidin-1-yl)pentyl)-5-(thiophen-2-yl)isoxazole-3-carboxamide; N-(5-((3S,4R)-3-cyano-4-hydroxypyrrolidin-1-yl)pentyl)-5-(thiophen-2-yl)isoxazole-3-carboxamide, and N-(5-((3S,4S)-3-cyano-4-hydroxypyrrolidin-1-yl)pentyl)-5-(thiophen-2-yl)isoxazole-3-carboxamide; N-(5-((3R,4S)-3-carbamoyl-4-hydroxypiperidin-1-yl)pentyl)-5-(thiophen-2-yl)isoxazole-3-carboxamide and N-(5-((3R,4R)-3-carbamoyl-4-hydroxypiperidin-1-yl)pentyl)-5-(thiophen-2-yl)isoxazole-3-carboxamide; N-(5-(3-((4-hydroxytetrahydrofuran-3-yl)carbamoyl)azetidin-1-yl)pentyl)-5-(thiophen-2-yl)isoxazole-3-carboxamide; N-(5-(4-(3-amino-2-hydroxy-3-oxopropyl)piperazin-1-yl)pentyl)-5-(thiophen-2-yl)isoxazole-3-carboxamide.

12. The compound or a pharmaceutically acceptable salt thereof according to claim 11, wherein the N-(5-(3-carbamoyl-4-hydroxypyrrolidin-1-yl)pentyl)-5-(thiophen-2-yl)isoxazole-3-carboxamide is selected from the following:

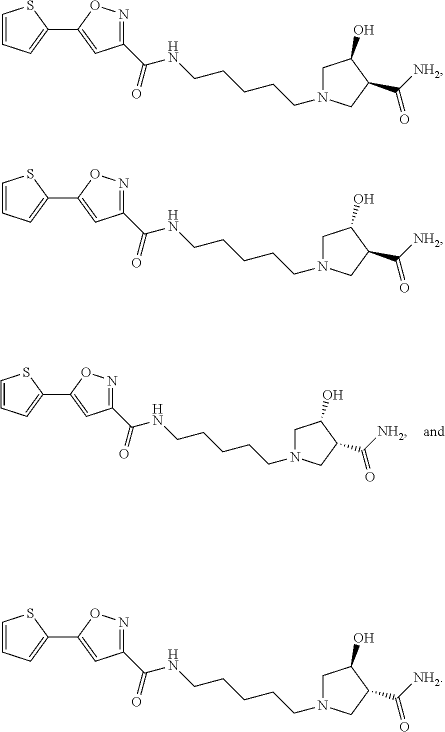 [SMILES: NC(=O)[C@H]1CN(CCCCCNC(=O)c2cc(-c3cccs3)on2)C[C@H]1O ; NC(=O)[C@H]1CN(CCCCCNC(=O)c2cc(-c3cccs3)on2)C[C@@H]1O ; NC(=O)[C@@H]1CN(CCCCCNC(=O)c2cc(-c3cccs3)on2)C[C@@H]1O ; "and" ; NC(=O)[C@@H]1CN(CCCCCNC(=O)c2cc(-c3cccs3)on2)C[C@H]1O]

13. A pharmaceutical composition, comprising: a therapeutically effective amount of a compound or a pharmaceutically acceptable salt thereof according to claim 1, and one or more pharmaceutically acceptable carriers.

14. A pharmaceutical combination, comprising: a therapeutically effective amount of a compound or a pharmaceutically acceptable salt thereof according to claim 1, and one or more therapeutically active co-agents.

15. The pharmaceutical combination of claim 14, wherein the co-agent is selected from active agents regulating Notch sigaling, FGF signaling, Wnt Signaling, Shh signaling, cell cycle/stem cell aging, miRNA and epigenetic regulations.

16. A method of treating hearing loss or a balance disorder, comprising administering to a subject in need thereof a therapeutically effective amount of a compound or a pharmaceutically acceptable salt thereof according to claim 1.
